(19) United States
(12) Patent Application Publication
(10) Pub. No.: US 2010/0016295 A1
(43) Pub. Date: Jan. 21, 2010

(54) Preparation and Use of Biphenyl Amino Acid Derivatives for the Treatment of Obesity

(75) Inventors: Roger A. Smith, Landenberg, PA (US); Derek Lowe, Hamden, CT (US); Tatiana Shelekhin, Ridgefield, CT (US); Georgiy Bondar, West Haven, CT (US); Philip Coish, North Haven, CT (US); Stephen J. O'Connor, Guiford, CT (US)

Correspondence Address: Barbara A. Shimei;Director, Patents & Licensing, Bayer HealthCare LLC - Pharmaceuticals, 555 White Plains Road, Third Floor, Tarrytown, NY, 10591, US

(73) Assignee: Bayer Healthcare LLC, Tarrytown, NY (US)

(21) Appl. No.: 11/989,506

(22) PCT Filed: Jul. 31, 2006
(86) PCT No.: PCT/US06/29871

§ 371 Date: Sep. 16, 2009

Related U.S. Application Data
(60) Provisional application No. 60/703,754, filed on Jul. 29, 2005.

(51) Int. Cl. A61K 31/198; C07D 211/60; A61P 3/04; A61K 31/44; C07C 229/02; C07D 207/00; A61K 31/40; C07D 261/18; A61K 31/42; C07D 307/00; A61K 31/34; C07D 209/42; C07D 279/12; A61K 31/54; C07D 265/30; A61K 31/5375; C07D 241/04; A61K 31/4965

(52) U.S. Cl. 514/227.5; 546/245; 514/562; 514/561; 514/354; 562/553; 548/532; 514/423; 548/248; 514/378; 549/468; 514/469; 548/492; 514/419; 544/59; 544/106; 514/237.5; 544/389; 514/255.01

(57) ABSTRACT

This invention relates to certain biphenyl amino acid compounds, compositions, and methods for treating or preventing obesity and related diseases.

This application claims benefit of U.S. Provisional Application Ser. No. 60/703,754, filed Jul. 29, 2005, the contents of which are incorporated herein by reference in their entirety.

### FIELD OF THE INVENTION

This invention relates to certain biphenyl amino acid compounds, compositions, and methods for treating or preventing obesity and related diseases.

### BACKGROUND OF THE INVENTION

Obesity, which is an excess of body fat relative to lean body mass, is a chronic disease that is highly prevalent in modern society. It is associated not only with a social stigma, but also with decreased life span and numerous medical problems, including adverse psychological development, coronary artery disease, hypertension, stroke, diabetes, hyperlipidemia, and some cancers (see, e.g., Nishina, et al., Metab. 43:554-558, 1994; Grundy and Barnett, Dis. Mon. 36:641-731, 1990; Rissanen, et al., British Medical Journal, 301:835-837, 1990).

Obesity remains a problem, and treatment has been limited. There is, therefore, a need to develop pharmaceuticals and treatment regimes effective in the alleviation of obesity.

A hallmark characteristic of obesity is an increase in white adipose tissue (WAT) mass that is largely due to accumulation of triacylglycerol. This increase in WAT mass is a key contributor to obesity-associated complications. Diacylglycerol O-acyltransferases (DGATs, EC 2.3.1.2) are membrane-bound enzymes that catalyze the terminal step of triacylglycerol biosynthesis. Two enzymes that display DGAT activity have been characterized: DGAT-1 (diacylglycerol O-acyltransferase type 1) (see, e.g., U.S. Pat. No. 6,100,077; Cases, et al., Proc. Nat. Acad. Sci. 95:13018-13023, 1998) and DGAT-2 (diacylglycerol O-acyltransferase type 2) (Cases, et al., J. Biol. Chem. 276:38870-38876, 2001). DGAT-1 and DGAT-2 do not exhibit significant protein sequence identity. Importantly, DGAT-1 null mice do not become obese when challenged with a high fat diet in contrast to wild-type littermates (Smith, et al., Nature Genetics 25:87-90, 2000). DGAT-1 null mice display reduced postprandial plasma glucose levels and exhibit increased energy expenditure, but have normal levels of serum triglycerides (Smith, et al., 2000), possibly due to the preserved DGAT-2 activity. Since DGAT-1 is expressed in the intestine and adipose tissue (Cases, et al., 1998), there are at least two possible mechanisms to explain the resistance of DGAT-1 null mice to diet-induced obesity. First, abolishing DGAT-1 activity in the intestine may block the reformation and export of triacylglycerol from intestinal cells into the circulation via chylomicron particles. Second, knocking out DGAT-1 activity in the adipocyte may decrease deposition of triacylglycerol in WAT. The phenotype of the DGAT-1 null mouse, along with the results of our studies with DGAT-1 inhibitors in diet-induced obese (DIO) mice, indicate that a DGAT-1 inhibitor has utility for the treatment of obesity and obesity-associated complications.

### DETAILED DESCRIPTION OF THE INVENTION

The invention relates to biphenyl amino acid derivatives, and pharmaceutical salts and esters thereof, that have utility in the inhibition of DGAT-1 (diacylglycerol O-acyltransferase type 1) and in the treatment of obesity and related diseases.

One embodiment of the invention is a compound of Formula (I)

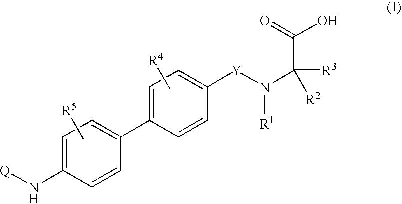

wherein
Y is C═O or S(═O)₂;
R¹ is hydrogen or (C₁-C₆)alkyl; is (C₁-C₆)alkyl, hydroxy-(C₁-C₆)alkyl, (C₁-C₆)alkoxy-C₁₋₆)alkyl, amino-(C₁-C₆)alkyl, (C₁-C₆)alkylamino-(C₁-C₆)alkyl, or bis[(C₁-C₆)alkyl]amino-(C₁-C₆)alkyl;
R³ is hydrogen; or
R¹ is hydrogen or (C₁-C₆)alkyl;
R² is R⁶(CH₂)ₘ,
   wherein
      m is 0 to 3,
      R⁶ is phenyl optionally substituted with one or more halogen, hydroxy, (C₁-C₆)alkyl, (C₁-C₆)alkoxy, trifluoromethyl, cyano, or nitro, or
      R⁶ is 2-pyridinyl, 3-pyridinyl, or 4-pyridinyl, each of which is optionally substituted with halogen, (C₁-C₆)alkyl, (C₁-C₆)alkoxy, trifluoromethyl, cyano, or nitro;
R³ is hydrogen; or
R¹ is hydrogen or (C₁-C₆)alkyl;
R² and R³ are identical and are each selected from (C₁-C₆)alkyl; or
R² and R³, together with the carbon to which they are attached, form a three- to six-membered carbocyclic ring; or
R¹ and R², together with the atoms to which R¹ and R² are attached, form a five- to seven-membered pyrrolidinyl-, piperidinyl-, or homopiperidinyl ring, or form a ring fragment selected from

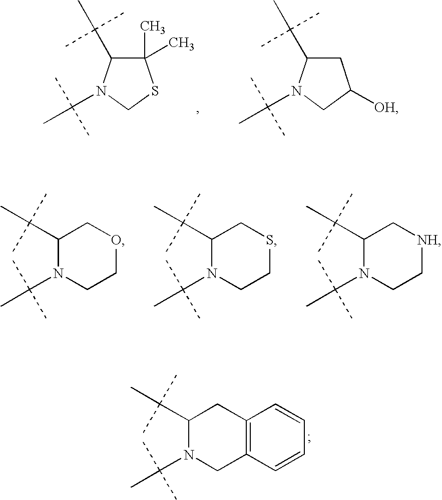

R³ is hydrogen;
R⁴ and R⁵ are independently selected from hydrogen, halogen, (C₁-C₆)alkyl, (C₁-C₆)alkoxy, hydroxy, trifluoromethyl, and cyano;
Q is R⁷—C(═O)—,
   wherein
      R⁷ is (C₁-C₆)alkyl optionally substituted with one or more hydroxy, (C₁-C₆)alkoxy, bis[(C₁-C₆)alkyl)]amino, or fluoro, or
      R¹ is R⁸(CH₂)ₙ,
         wherein
         n is 0 to 3,
         R⁸ is phenyl optionally substituted with one or more halogen, hydroxy, (C₁-C₆)alkyl, (C₁-C₆)alkoxy, trifluoromethyl, cyano, or nitro, or
         R⁸ is 2-pyridinyl, 3-pyridinyl, or 4-pyridinyl, each of which is optionally substituted with halogen, (C₁-C₆)alkyl, (C₁-C₆)alkoxy, trifluoromethyl, cyano, or nitro; or
      R⁷ is R¹⁰C(R⁹)₂,
         wherein
         R⁹ is methyl or ethyl, or
         C(R⁹)₂ is a 1,1-cyclopropyl, 1,1-cyclobutyl, 1,1-cyclopentyl, or 1,1-cyclohexyl ring,
         R¹⁰ is phenyl optionally substituted with one or more halogen, hydroxy, (C₁-C₆)alkyl, (C₁-C₆)alkoxy, trifluoromethyl, cyano, or nitro, or
         R¹⁰ is 2-pyridinyl, 3-pyridinyl, or 4-pyridinyl, each of which is optionally substituted with halogen, (C₁-C₆)alkyl, (C₁-C₆)alkoxy, trifluoromethyl, cyano, or nitro; or
      R⁷ is a fragment group selected from

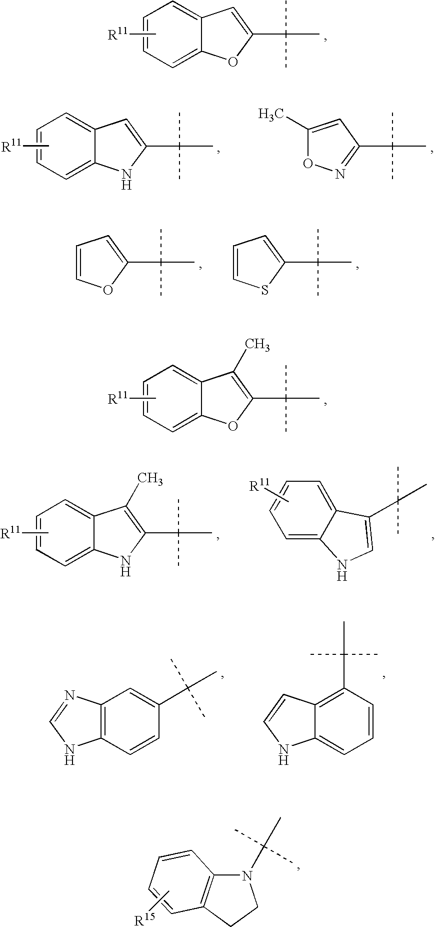

wherein
R¹¹ is one or more substituents selected from hydrogen, halogen, hydroxy, (C₁-C₆)alkyl, (C₁-C₆)alkoxy, trifluoromethyl, cyano, and nitro; or
Q is R¹³—N(R¹²)—C(═O)—,
   wherein
      R¹² is hydrogen or (C₁-C₆)alkyl,
      R¹³ is (C₁-C₆)alkyl optionally substituted with one or more hydroxy, (C₁-C₆)alkoxy, bis[(C₁-C₆)alkyl)]amino, or fluoro; or
      R¹³ is R¹⁷(CH₂)ₚ,
         wherein
         p is 0 to 3,
         R¹⁷ is phenyl optionally substituted with one or more halogen, hydroxy, (C₁-C₆)alkyl, (C₁-C₆)alkoxy, trifluoromethyl, trifluoromethoxy, cyano, or nitro, or
         R¹⁷ is 2-pyridinyl, 3-pyridinyl, or 4-pyridinyl, each of which is optionally substituted with halogen, (C₁-C₆)alkyl, (C₁-C₆)alkoxy, trifluoromethyl, cyano, or nitro; or
      R¹² and R¹³ and the nitrogen atom to which they are attached form a ring fragment, selected from

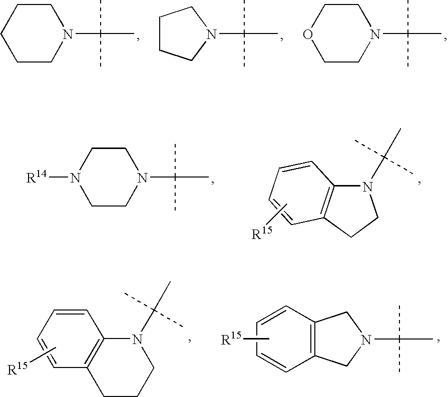

wherein
   R¹⁴ is (C₁-C₆)alkyl; or
   R¹⁴ is R¹⁶(CH₂)_{q},
      wherein
      q is 0 or 1,
      R¹⁶ is phenyl optionally substituted with one or more halogen, hydroxy, (C₁-C₆)alkyl, (C₁-C₆)alkoxy, trifluoromethyl, trifluoromethoxy, cyano, or nitro, or
      R¹⁶ is 2-pyridinyl, 3-pyridinyl, or 4-pyridinyl, each of which is optionally substituted with halogen, (C₁-C₆)alkyl, (C₁-C₆)alkoxy, trifluoromethyl, cyano, or nitro;
R¹⁵ is one or more substituents selected from halogen, hydroxy, (C₁-C₆)alkyl, (C₁-C₆)alkoxy, trifluoromethyl, cyano, and nitro; or pharmaceutically acceptable salts and esters thereof,
   with the proviso that Formula (I) is not N-([4′-(2-methoxy-acetylamino)-1,1′-biphenyl-4-yl]-carbonyl)-L-phenylalanine.

Examples of the invention may be found in the Examples described below and in the Tables. The compounds described in the Examples are intended to be representative of the invention, and it will be understood that the scope of the invention is not limited by the scope of the examples. Those skilled in the art will recognize that the invention may be practiced with variations on the disclosed structures, materials, compositions and methods, and such variations are regarded as within the ambit of the invention.

The terms identified above have the following meaning throughout:

The term “halogen” means P, Br, Cl, and I.

The term “(C₁-C₆)alkyl” means a linear or branched saturated hydrocarbon group having from about 1 to about 6 carbon atoms. The hydrocarbon group may also include a cyclic alkyl radical as part of the alkyl group. Such groups include, but are not limited to, methyl, ethyl, n-propyl, isopropyl, butyl, isobutyl, pentyl hexyl, cyclopropyl, cyclohexyl, cyclopropyl-methyl, and cyclopentyl-methyl groups.

The term “(C₁-C₆)alkoxy” means a linear or branched saturated hydrocarbon group having from about 1 to about 6 carbon atoms, said group being attached to an oxygen atom. The oxygen atom is the atom through which the alkoxy substituent is attached to the rest of the molecule. The hydrocarbon group may also include a cyclic alkyl radical as part of the alkyl group. Such groups include, but are not limited to, methoxy, ethoxy, n-propoxy, isopropoxy, n-butoxy, n-hexyloxy, 3,3-dimethylpropoxy, cyclopropoxy, cyclopropylmethoxy, cyclopentyloxy, and the like.

The term “three- to six-membered carbocyclic ring” means a saturated or partially unsaturated ring containing from about 3 to about 6 carbon atoms. Such groups include, but are not limited to, cyclopropyl, cyclobutyl, cyclopentyl cyclohexyl, cyclopentenyl, cyclohexenyl, and the like.

The term “hydroxy-(C₁-C₆)alkyl” means a (C₁-C₆)alkyl group, said alkyl being further substituted by a hydroxy group at any available carbon atom. Such groups include, but are not limited to, hydroxymethyl, 2-hydroxyethyl, 2 hydroxypropyl, 3 hydroxypropyl, 4-hydroxybutyl, 2-hydroxy-1-methylethyl, 5-hydroxypentyl, 3-hydroxybutyl, 3-hydroxy-2-ethylpropyl, 6-hydroxyhexyl, and the like.

The term “optionally substituted” means that the moiety so modified may have from none to up to at least the highest number of substituents indicated. Each substituent may replace any hydrogen atom on the moiety so modified as long as the replacement is chemically possible and chemically stable. When there are two or more substituents on any moiety, each substituent is chosen independently of any other substituent and can, accordingly, be the same or different.

When any moiety is described as being substituted, it can have one or more of the indicated substituents that can be located at any available position on the moiety. When there are two or more substituents on any moiety, each term shall be defined independently of any other in each occurrence.

Representative salts of the compounds of Formula (I) include the conventional non-toxic salts and the quaternary ammonium salts which are formed, for example, from inorganic or organic acids or bases by means well known in the art. For example, such acid addition salts include acetate, adipate, alginate, ascorbate, aspartate, benzoate, benzenesulfonate, bisulfate, butyrate, citrate, camphorate, camphorsulfonate, cinnamate, cyclopentanepropionate, digluconate, dodecylsulfate, ethanesulfonate, fumarate, glucoheptanoate, glycerophosphate, hemisulfate, heptanoate, hexanoate, hydrochloride, hydrobromide, hydroiodide, 2-hydroxyethanesulfonate, itaconate, lactate, maleate, mandelate, methanesulfonate, 2-naphthalenesulfonate, nicotinate, nitrate, oxalate, pamoate, pectinate, persulfate, 3-phenylpropionate, picrate, pivalate, propionate, succinate, sulfonate, tartrate, thiocyanate, tosylate, and undecanoate.

Base salts include alkali metal salts such as potassium and sodium salts, alkaline earth metal salts such as calcium and magnesium salts, and ammonium salts with organic bases such as dicyclohexylamine salts and N-methyl-D-glucamine. Additionally, basic nitrogen containing groups may be quaternized with such agents as lower alkyl halides such as methyl, ethyl, propyl, and butyl chlorides, bromides and iodides; dialkyl sulfates like dimethyl, diethyl, and dibutyl sulfate; and diamyl sulfates, long chain halides such as decyl lauryl, myristyl and stearyl chlorides, bromides and iodides, aralkyl halides like benzyl and phenethyl bromides and others.

The esters in the present invention are non-toxic, pharmaceutically acceptable ester derivatives of the compounds of Formula (I). This includes, for example, ester derivatives of hydroxy-containing compounds of Formula (I) prepared with acetic, benzoic, mandelic, stearic, lactic, salicylic, hydroxynaphthoic, glucoheptonic, and gluconic acid. This also includes, for example, ester derivatives of carboxylic acid-containing compounds of Formula (I) prepared with pharmaceutically acceptable alcohols. Pharmaceutically acceptable alcohols include, but are not limited to methanol, ethanol, isopropanol butanol, 2-methylpropanol, 2-methoxyethanol, 2-(dimethylamino)ethanol, 2-(diethylamino)ethanol, 2-(1-piperidinyl)ethanol, 2-(1-morpholinyl)ethanol, hydroxyacetic acid, N,N-dimethylglycolamide, hydroxyacetone, and the like. The compounds of Formula (I) having carboxylic acid groups may be esterified by a variety of conventional procedures well known by those skilled in the art. One skilled in the art would readily know how to successfully carry out these as well as other methods of esterification.

Sensitive or reactive groups on the compounds of Formula (I) may need to be protected during any of the above methods for forming esters, and protecting groups may be added and removed by conventional methods well known in the art.

The compounds of this invention may, either by nature of asymmetric centers or by restricted rotation, be present in the form of isomers. Any isomer may be present in which the asymmetric center is in the (R)-, (S)-, or (R,S) configuration.

It will also be appreciated that when two or more asymmetric centers are present in the compounds of the invention, that several diastereomers and enantiomers of the exemplified structures will often be possible, and that pure diastereomers and pure enantiomers represent preferred embodiments. It is intended that pure stereoisomers, pure diastereomers, pure enantiomers, and mixtures thereof, are within the scope of the invention.

AR isomers, whether separated, pure, partially pure, or in racemic mixture, of the compounds of this invention are encompassed within the scope of this invention. The purification of said isomers and the separation of said isomeric mixtures may be accomplished by standard techniques known in the art.

Geometric isomers by nature of substituents about a double bond or a ring may be present in cis (=Z) or trans (=E-) form, and both isomeric forms are encompassed within the scope of this invention.

The particular process to be utilized in the preparation of the compounds of this invention depends upon the specific compound desired. Such factors as the selection of the specific moieties and the specific substituents on the various moieties, all play a role in the path to be followed in the preparation of the specific compounds of this invention. These factors are readily recognized by one of ordinary skill in the art.

For synthesis of any particular compound, one skilled in the art will recognize that the use of protecting groups may be required for the synthesis of compounds containing certain substituents. A description of suitable protecting groups and appropriate methods of adding and removing such groups may be found, for example, in Protective Groups in Organic Synthesis, Second Edition, T. W. Greene, John Wiley and Sons, New York, 1991.

In the reaction schemes below, one skilled in the art will recognize that reagents and solvents actually used may be selected from several reagents and solvents well known in the art to be effective equivalents. When specific reagents or solvents are shown in a reaction scheme, therefore, they are meant to be illustrative examples of conditions desirable for the execution of that particular reaction scheme. Abbreviations not identified in accompanying text are listed later in this disclosure under “Abbreviations and Acronyms.”

Another object of this invention is to provide methods of making the compounds of the invention. The compounds may be prepared from readily available materials by the methods outlined in the reaction schemes and Examples below, and by obvious modifications thereto.

### General Preparation of Compounds of the Invention

Preparation of the Compounds of the Present Invention Having Formula (I), May be accomplished by the general methods shown below in Reaction Schemes 1 to 3.

In Reaction Scheme 1, a biphenyl carboxylic acid or sulfonic acid is nitrated, and the corresponding acid chloride (when Y is C═O) or sulfonyl chloride (when Y is S(═O)₂ is prepared using, for example, oxalyl chloride. This intermediate is coupled with suitably functionalized and protected amino acid esters, which are commercially available or can be prepared from their amino acid precursors by well known methods. The amino acid ester can be a methyl ester as indicated in Scheme 1, and it is well known by those skilled in the art that other esters such as ethyl, tert-butyl and benzyl can also be used. The coupling reaction with the amino acid ester is typically performed in the presence of a non-nucleophilic base such as diisopropylethylamine. The resulting carboxamide (when Y is C═O) or sulfonamide (when Y is S(═O)₂) is then reduced to the p-amino-biphenyl derivative of Formula (II) by the use of iron in acetic acid. Numerous other methods for the formation of amides and the reduction of aryl nitro compounds are also well known in the art.

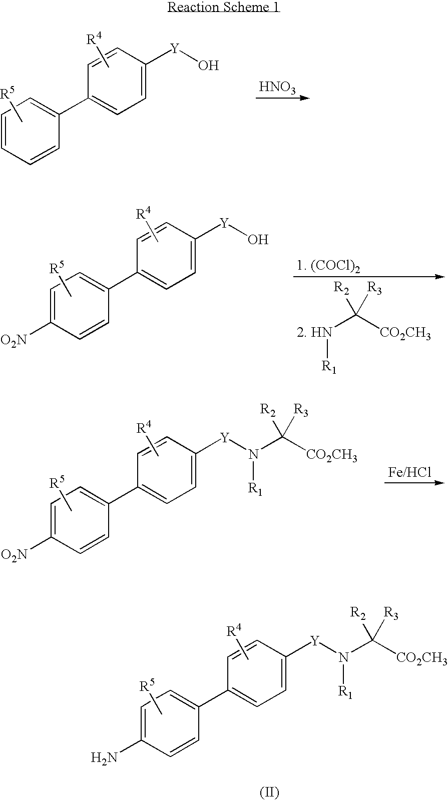

An alternative approach for the preparation of compounds of Formula (II) is shown in Reaction Scheme 2. A para-bromo-benzoyl chloride (when Y is C═O) or para-bromo-phenylsulfonyl chloride (when Y is S(═O)₂) is reacted with an amino acid ester (as used in Reaction Scheme 1), in the presence of an aqueous or non-aqueous base. The resulting carboxamide (when Y is CEO) or sulfonamide (when Y is S(═O)₂) is coupled with a para-nitrophenylboronic acid under palladium catalysis (known as the Suzuki reaction), a wide range of conditions for which are well known in the art. The nitro group of the resulting biphenyl derivative is reduced as in the previous reaction scheme to provide the ester of Formula (II).

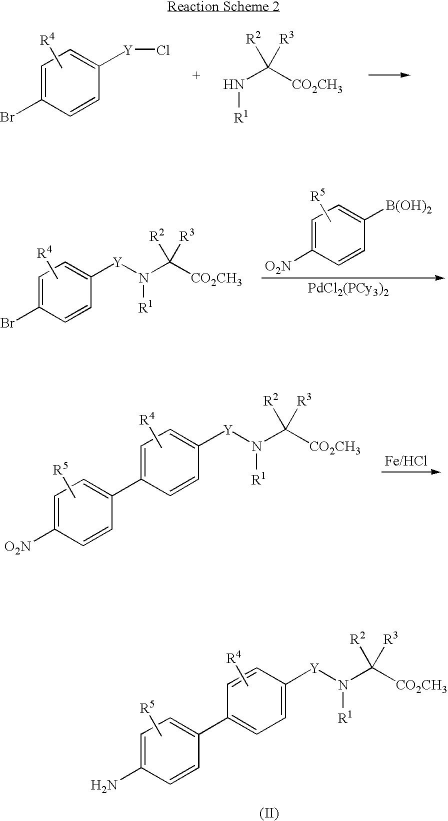

The compounds of Formula (II) are then converted to a compound of Formula (I) by one of the methods described in Reaction Scheme 3. For example, a compound of Formula (II) is allowed to react with a carboxylic acid chloride or fluoride, or with a carboxylic acid plus a coupling reagent such as N,N′-dicyclohexylcarbodiimide, to form the corresponding carboxamide, and then the ester group —COOR (for example, —COOCH₃ as indicated in the Reaction Schemes) is hydrolyzed under standard ester hydrolysis conditions to give a compound of Formula (Ia) (Formula (I) wherein Q is R⁷—C(═O)—).

Alternatively, the compound of Formula (II) is allowed to react with an isocyanate derivative, R¹³—N═C═O to form the corresponding urea derivative, and then the ester group —COOR (for example, —COOCH₃ as indicated in the Reaction Schemes) can be hydrolyzed under standard ester hydrolysis conditions to give a compound of Formula (Ib) (Formula (I) wherein Q is R¹³—NH—(C═O)—).

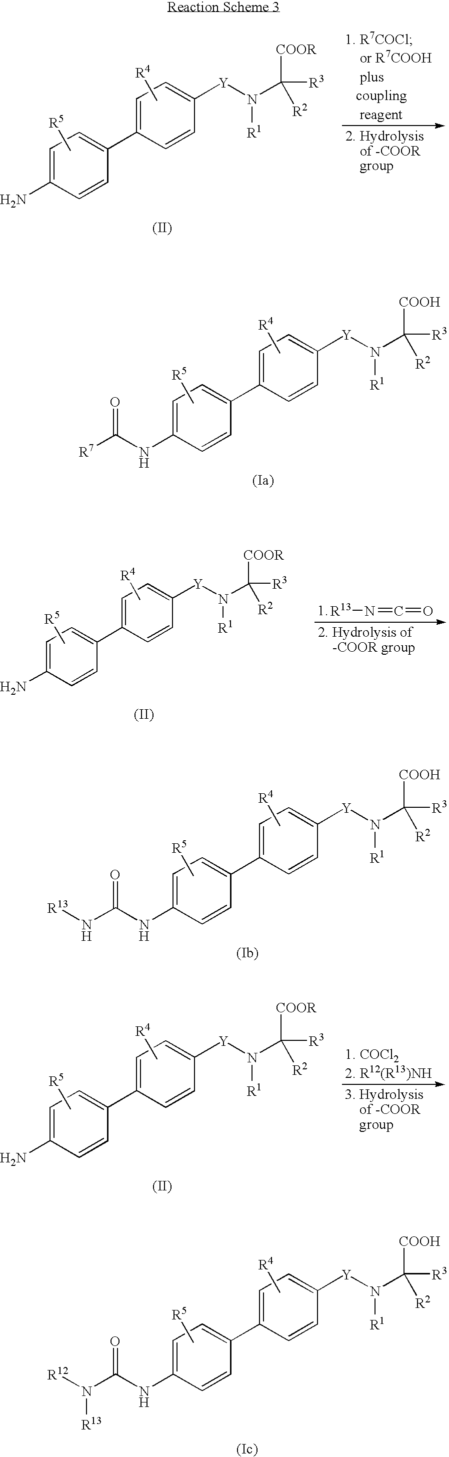

Also, the compound of Formula (II) can be reacted with phosgene or a substitute such as triphosgene to form an isocyanate intermediate, which is then reacted with a secondary amine (R¹²R¹³NH) to form the corresponding urea derivative. Then the ester group COOR can be hydrolyzed under standard ester hydrolysis conditions to give a compound of Formula (Ic) (Formula (I) wherein Q is R¹²—N(R¹³—)—(C═O)—).

Examples of the invention may be found in the Examples described below and in the Tables. The compounds described in the Examples are intended to be representative of the invention, and it will be understood that the scope of the invention is not limited by the scope of the examples. Those skilled in the art will recognize that the invention may be practiced with variations on the disclosed structures, materials, compositions and methods, and such variations are regarded as within the ambit of the invention.

### PREPARATION OF COMPOUNDS OF THE INVENTION

### Analytical Methods

### Mass Spectra

Chemical ionization mass spectra (CI-MS) were obtained with a Hewlett Packard 5989A mass spectrometer equipped with a Hewlett Packard 5890 Gas Chromatograph with a J & W DB-5 column (0.25 uM coating; 30 m×0.25 mm). The ion source was maintained at 250° C. and spectra were scanned from 50-800 amu at 2 sec per scan.

### Liquid Chromatography-Electrospray Mass Spectra

Liquid chromatography-electrospray mass spectra (LC-MS) data were obtained by using one of the following two methods. In the Examples and Tables provided below, the LC-MS data are given with HPLC retention times (ret. time). Except as noted otherwise, Method 1 was used.

Method 1: Hewlett-Packard 1100 HPLC equipped with a quaternary pump, a variable wavelength detector set at 254 nm, a YMC pro C-18 column (2×23 mm, 120A), and a Finnigan LCQ ion trap mass spectrometer with electrospray ionization. Spectra were scanned from 120-1200 amu using a variable ion time according to the number of ions in the source. The eluants were A: 2% acetonitrile in water with 0.02% TFA, and B: 2% water in acetonitrile with 0.018% TFA. Gradient elution from 10% B to 95% B over 3.5 minutes at a flow rate of 1.0 mL/min was used with an initial hold of 0.5 minutes and a final hold of 0.5 minutes at 95% B. Total run time was 6.5 minutes.

Method 2: Gilson HPLC system equipped with two Gilson 306 pumps, a Gilson 215 Autosampler, a Gilson diode array detector, a YMC Pro C-18 column (2×23 mm, 120 A), and a Micromass LCZ single quadrupole mass spectrometer with z-spray electrospray ionization. Spectra were scanned from 120-800 amu over 1.5 seconds. ELSD (Evaporative Light Scattering Detector) data was also acquired as an analog channel. The eluants were A: 2% acetonitrile in water with 0.02% TFA, and B: 2% water in acetonitrile with 0.018% TFA Gradient elution from 10% B to 90% B over 3.5 minutes at a flow rate of 1.5 mL/min was used with an initial hold of 0.5 minutes and a final hold of 05 minutes at 90% B. Total run time was 4.8 minutes. An extra switching valve was used for column switching and regeneration.

### NMR Spectra

Routine one-dimensional NMR spectroscopy was performed on 300 MHz or 400 MHz Varian Mercury-plus spectrometers. The samples were dissolved in deuterated solvents obtained from Cambridge Isotope Labs, and transferred to 5 mm ID Wilmad NMR tubes. The spectra were acquired at 293° K. The chemical shifts were recorded on the ppm scale and were referenced to the appropriate solvent signals, such as 2.49 ppm for DMSO-d₆, 1.93 ppm for CD₃CN, 3.30 ppm for CD₃OD, 5.32 ppm for CD₂Cl₂, and 7.26 ppm for CDCl₃ for ¹H spectra; and 39.5 ppm for DMSO-d₆, 1.3 ppm for CD₃CN, 49.0 ppm for CD₃OD, 53.8 ppm for CD₂Cl₂ and 77.0 ppm for CDCl₃ for ¹³C spectra.

### ABBREVIATIONS AND ACRONYMS

When the following abbreviations are used throughout the disclosure, they have the following meaning:

CDCl₃ deuterated chloroform
Celite® diatomaceous earth filter agent, ®Celite Corp.
DMSO dimethyl sulfoxide
DMSO-d₆ deuterated dimethyl sulfoxide
EtOAc ethyl acetate
h hour(s)
HPLC high pressure liquid chromatography
LC-MS liquid chromatography-mass spectrometry
MeOH methanol
min minutes
MS mass spectroscopy
m/z mass-to-charge ratio
NMR nuclear magnetic resonance
PdCl₂(dppf) 1,1′-bis(diphenylphosphino)ferrocene]dichloropalladium(II)
p.o. orally administered
rt room temperature
TFA trifluoroacetic acid

### PREPARATIVE EXAMPLES OF THE INVENTION

### Preparation of methyl N-[(4′-aminobiphenyl-4-yl)carbonyl]-N-methyl-L-valinate

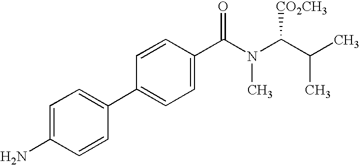

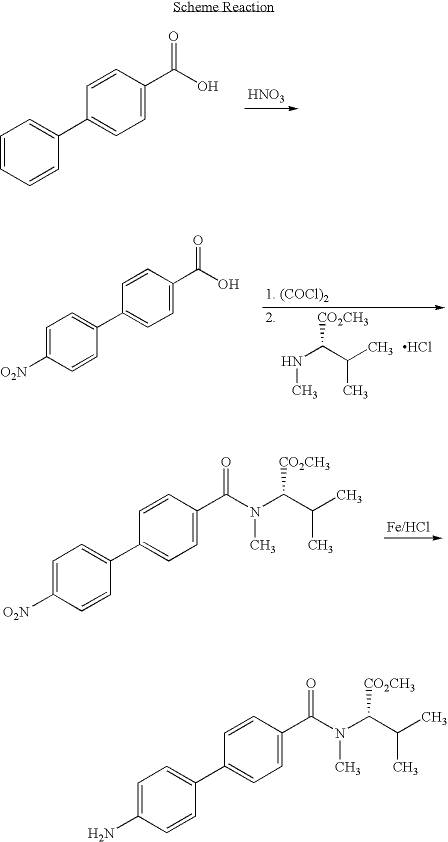

### Step 1. Preparation of 4′-nitro-1,1′-biphenyl-4-carboxylic acid

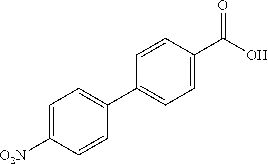

To ice-cold nitric acid was added 4-biphenylcarboxylic acid (9.4 g, 20.0 mmol), and the resulting mixture was stirred on ice for 1 h. The mixture was poured into ice water and filtered. The collected solid was suspended in ethanol and refluxed for 2 h. The mixture was filtered hot, washed with ethanol, and dried under high vacuum to give 4′-nitro-1,1′-biphenyl-4-carboxylic acid (2.3 g, 47%). ¹H NMR (400 MHz, DMSO-d₆) δ 7.89 (d, 2H), 8.01 (d, 2H), 8.05 (d, 2H), 8.31 (d, 2H), 13.12 (s, 1H).

### Step 2. Preparation of methyl N-methyl-N-[(4′-nitro-1,1′-biphenyl-4-yl)carbonyl]-L-valinate

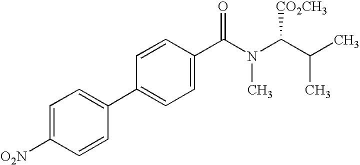

4′-Nitro-1,1′-biphenyl-4-carboxylic acid (0.50 g, 2.0 mmol) was dissolved in methylene chloride (25 mL) and oxalyl chloride (0.27 mL, 3.1 mmol) was added, followed by 1 drop of N,N-dimethyl-formamide. The resulting mixture was heated at 50° C. for 1 h, concentrated under reduced pressure, and further dried under vacuum for 30 min. The residue was dissolved in methylene chloride (20 mL) and added dropwise to an ice-cold mixture of methyl N-methyl-L-valinate hydrochloride (0.48 g, 2.6 mmol), methylene chloride (50 mL), and triethylamine (1.44 mL, 10.2 mmol). The resulting solution was stirred on ice for 1 h and then at rt overnight. The mixture was diluted with methylene chloride and washed with 1N aqueous hydrochloric acid solution and brine. The organic layer was separated and concentrated under reduced pressure. The residue was purified by flash chromatography (Biotage®) eluted with hexanes/ethyl acetate (3:1) to afford methyl N-methyl-N-[(4′-nitro-1,1′-biphenyl-4-yl)carbonyl]-L-valinate (0.70 g, 92%). ¹H NMR (400 MHz, CDCl₃) δ 0.85 (dd, 3H), 1.06 (dd, 3H), 2.31 (m, 1H), 3.02 (d, 3H), 3.77 (d, 3H), 3.94 (d, 0.5H), 4.98 (d, 0.5H), 7.52 (m, 2H), 7.64 (m, 2H, 7.72 (t, 2H), 8.27 (d, 2H); LC-MS m/z 371.2 (MH+), retention time 3.27 minutes.

### Step 3. Preparation of methyl N-[(4′-amino-1,1′-biphenyl-4-yl)carbonyl]-N-methyl-L-valinate

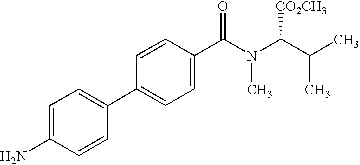

To a solution of N-methyl-N-[(4′-nitro-1,1′-biphenyl-4-yl)carbonyl]-L-valinate (0.70 g, 1.9 mmol) in 85% ethanol (20 mL) was added iron powder (1.05 g, 18.9 mmol) and 2N aqueous hydrochloric acid solution (0.41 mL). The resulting mixture was heated at reflux for 2 h. The mixture was then filtered through a pad of Celite® and concentrated under reduced pressure. The residue was dissolved in methylene chloride, washed with water and brine, dried (Na₂SO₄), and concentrated under reduced pressure to afford methyl N-[(4′-amino-1,1′-biphenyl-4-yl)carbonyl]-N-methyl-L-valinate (0.49 g, 76%). ¹H NMR (400 MHz, CD₃OD) δ 0.85 (dd, 3H), 1.06 (dd, 3H), 2.35 (m, 1H), 3.02 (d, 3H), 3.75 (d, 3H), 4.05 (d, 0.5H), 4.78 (d, 0.5H), 6.78 (d, 2H), 7.42 (m, 4H), 7.63 (d, 2H); LC-MS m/z 341.2 (MH+), retention time 2.37 minutes.

### Preparation of Methyl N-[(4′-amino-1,1′-biphenyl-4-yl)carbon-yl]-L-valinate

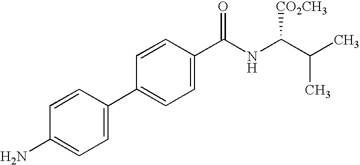

### Step 1. Preparation of methyl N-[(4′-nitro-1,1′-biphenyl-4-yl)carbonyl]-L-valinate

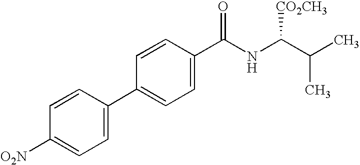

4′-Nitro-1,1′-biphenyl-4-carboxylic acid (0.60 g, 2.4 mmol) was dissolved in methylene chloride (25 mL) and oxalyl chloride (0.32 mL, 3.7 mmol) was added, followed by 1 drop of N,N-dimethylformamide. The resulting mixture was heated at 50° C. for 1 h, concentrated under reduced pressure, and further dried under vacuum for 30 min. The residue was dissolved in methylene chloride (20 mL) and added dropwise to an ice-cold mixture of methyl L-valinate hydrochloride (0.54 mg, 3.2 mmol), methylene chloride (25 mL), and triethylamine (1.74 mL, 12.3 mmol). The resulting solution was stirred on ice for 1 h and then at rt overnight The mixture was diluted with methylene chloride and washed with 1N aqueous hydrochloric acid solution and brine. The organic layer was separated and concentrated under reduced pressure. The residue was purified by flash chromatography (Biotage®) eluted with hexanes/ethyl acetate (2:1) to afford methyl N-[(4′-nitro-1,1′-biphenyl-4-yl)carbonyl]-L-valinate (0.70 g, 80%). ¹H NMR (400 MHz, CD₃OD) δ 1.06 (dd, 6H), 2.38 (m, 1H), 3.77 (s, 3H), 4.52 (d, 1H), 7.82 (d, 21H), 7.91 (d, 2H), 7.97 (d, 2H), 8.33 (d, 2H); LC-MS m/z 357.1 (MH+), retention time 3.07 minutes.

### Step 2. Preparation of methyl N-[(4′-amino-1,1′-biphenyl-4-yl)carbonyl]-L-valinate

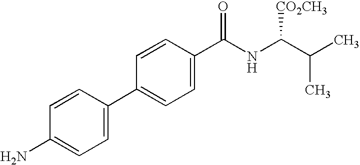

To a solution of methyl N-[(4′-nitro-1,1′-biphenyl-4-yl)carbonyl]-L-valinate (0.70 g, 1.9 mmol) in 85% ethanol (20 mL) was added iron powder (1.09 g, 19.6 mmol) and 2V aqueous hydrochloric acid solution (1.0 mL). The resulting mixture was heated at reflux for 2 h. The mixture was then filtered through a pad of Celite® and concentrated under reduced pressure. The residue was dissolved in dichloromethane and washed with water and brine, dried (Na₂SO₄), and concentrated under reduced pressure to afford methyl N-[(4′-amino-1,1′-biphenyl-4-yl)carbonyl]-L-valinate (0.58 g, 90%). ¹H NMR (400 MHz, CD₃OD) δ 1.03 (dd, 6H), 2.26 (m, 1H), 3.74 (s, 3H), 4.48 (d, 1H), 6.77 (d, 2H), 7.43 (d, 2H), 7.62 (d, 2H), 7.83 (d, 2H); LC-MS m/z 327.1 (MH+), retention time 2.27 minutes.

### Preparation of Methyl 2-methyl-N[(4′-aminobiphenyl-4-yl)carbonyl]alaninate

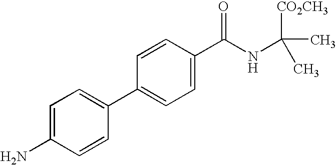

### Step 1. Preparation of methyl 2-methyl-N-[(4′-nitrobiphenyl-4-yl)carbonyl]alaninate

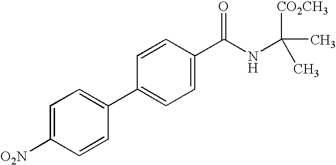

4′-Nitro-1,1′-biphenyl-4-carboxylic acid (4.66 g, 19.2 mmol) was dissolved in methylene chloride (110 mL) and oxalyl chloride (2.51 mL, 28.7 mmol) was added, followed by 3 drops of N,N-dimethylformamide. The resulting mixture was stirred at rt for 45 min, concentrated under reduced pressure, and further dried under vacuum for 30 min. The residue was dissolved in methylene chloride (75 mL) and added dropwise to an ice-cold mixture of methyl 2-methylalaninate hydrochloride (3.83 g, 24.9 mmol), methylene chloride (75 mL), and triethylamine (6.68 mL, 47.9 mmol). The resulting solution was stirred at rt for 1 h and then at 55° C. for 2 h. The mixture was allowed to cool to rt and was washed with 1N aqueous hydrochloric acid solution (5 mL) and water (2×20 mL). The organic layer was separated, dried (MgSO₄), and concentrated under reduced pressure. The residue was purified by flash chromatography (Biotage®) eluted with hexanes/ethyl acetate (4:1) to afford methyl 2-methyl-N-[(4′-nitrobiphenyl-4-yl)carbonyl]alaninate (6.21 g, 95%). ¹H NMR (400 MHz, CDCl₃) δ 1.73 (s, 6H), 3.82 (s, 3H), 6.89 (broad s, 1H), 7.69 (d, 2H), 7.77 (d, 2H), 7.92 (d, 2H), 8.31 (d, 2H); LC-MS m/z 342.9 (MH+), retention time 2.98 minutes.

### Step 2. Preparation of methyl N-[(4′-aminobiphenyl-4-yl)carbonyl]-2-methylalaninate

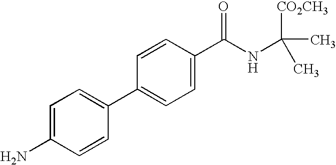

To a solution of methyl 2-methyl-N-[(4′-nitrobiphenyl-4-yl)carbonyl]alaninate (1.59 g, 4.6 mmol) in 85% ethanol (50 mL) was added iron powder (2.59 g, 46.4 mmol) and 2M aqueous hydrochloric acid solution (2.32 mL, 4.6 mmol). The resulting mixture was heated at reflux for 2 h. The mixture was then filtered through a pad of Celite) and concentrated under reduced pressure to afford methyl N-[(4′-aminobiphenyl-4-yl)carbonyl]-2-methylalaninate as a yellow solid (2.48 g, 99%). ¹H NMR (400 MHz, DMSO-d₆) δ 1.44 (s, 6H), 3.57 (s, 3H), 5.33 (broad s, 2H), 6.61 (d, 2H), 7.41 (d, 2H), 7.58 (d, 2H), 7.83 (d, 2H), 8.54 (broad s, 1H); LC-MS m/z 313.2 (MH+), retention time 1.54 minutes.

### Preparation of Methyl N-[(4′-aminobiphenyl-4-yl)carbonyl]-N,2-dimethylalaninate

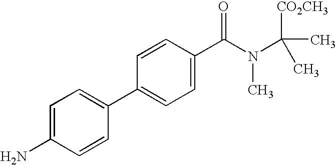

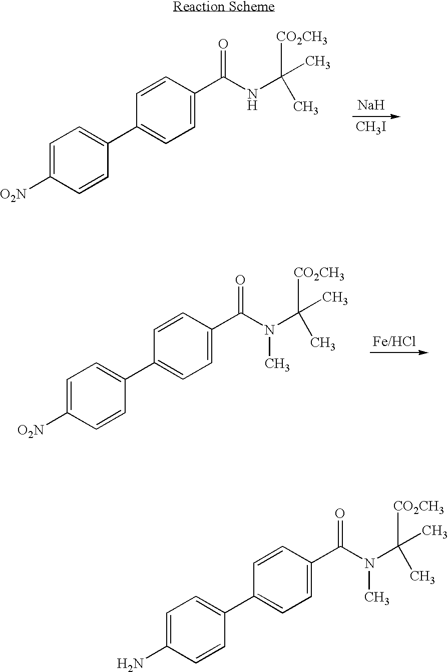

### Step 1. Preparation of methyl N,2-dimethyl-N-[(4′-nitrobiphenyl-4-yl)carbonyl]alaninate

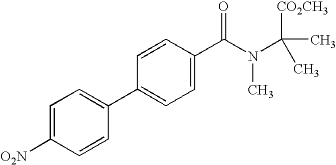

A mixture of methyl 2-methyl-N-[(4′-nitrobiphenyl-4-yl)carbonyl]alaninate (1.32 g, 3.9 mmol), sodium hydride (117 mg, 4.6 mmol), and N,N-dimethylformamide (15 mL) was stirred for 2 h at rt. Iodomethane (0.48 mL, 7.7 mmol) was added, and the reaction mixture was stirred overnight at rt. Water (30 mL) was added, and the mixture was extracted with ethyl acetate (2×10 mL). The combined extracts were evaporated to dryness, and crude product was purified by flash chromatography (Biotage®.), and eluted with 4:1 hexanes/ethyl acetate to yield methyl N,2-dimethyl-N-[(4′-nitrobiphenyl-4-yl)carbonyl]alaninate as off-white solid (1.29 g, 94%). ¹H NMR (400 MHz, DMSO-d₆) δ 1.47 (s, 6H), 2.93 (s, 3H), 3.58 (s, 3H), 7.52 (d, 2H), 7.84 (d, 2H), 7.98 (d, 2H), 8.30 (d, 2H); LC-MS m/z 356.9 (MH+), retention time 2.98 minutes.

### Step 2. Preparation of methyl N-[(4′-aminobiphenyl-4-yl)carbonyl]-N,2-dimethylalaninate

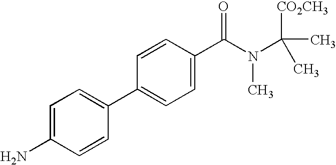

To a solution of methyl N,2-dimethyl-N-[(4′-nitrobiphenyl-4-yl)carbonyl]alaninate (1.92 g, 5.4 mmol) in 85% ethanol (50 mL) was added iron powder (3.01 g, 53.88 mmol) and 2M aqueous hydrochloric acid solution (2.69 μL, 5.4 mmol). The resulting mixture was heated at reflux for 2.5 h. The mixture was filtered through a pad of Celite® and concentrated under reduced pressure to afford methyl N-[(4′-aminobiphenyl-4-yl)carbonyl]-N,2-dimethylalaninate as a yellow solid (1.44 g, 82%). ¹H NMR (400 MHz, DMSO-d₆) 1.42 (s, 6H), 2.94 (s, 3H), 3.57 (s, 3H), 5.30 (broad s, 2H), 6.61 (d, 2H), 7.31-7.40 (m, 4H), 7.58 (d, 2H); LC-MS m/z 327.2 (MH+), retention time 1.84 minutes.

### Preparation of Methyl N-[(4′-amino-1,1′-biphenyl-4-yl)sulfonyl]-L-prolinate

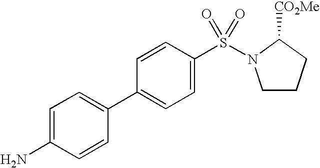

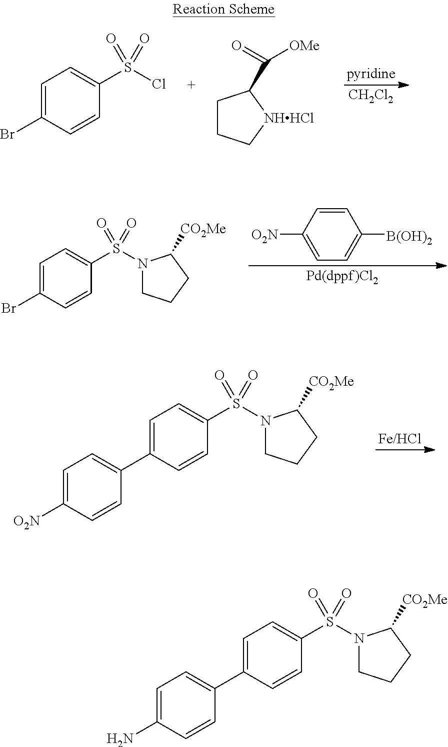

### Step 1. Preparation of methyl N-[(4-bromophenyl)sulfonyl]-prolinate

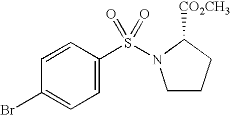

L-proline (2.00 g, 12.0 mmol) was suspended in methylene chloride (50 mL) and pyridine (4.88 mL, 60.0 mmol). The resulting mixture was cooled to 0° C. and a solution of bromophenyl sulphonyl chloride (4.63 g, 18.0 mmol) in methylene chloride (20 mL) was added dropwise over a 20-minute period. The mixture was removed from the cold bath and allowed to stir at rt overnight. Volatile components were removed by rotary evaporation, and the residue was partitioned between methylene chloride and water. The organic layer was separated, washed with 1N aqueous hydrochloric acid solution, water, and brine. The organic layer was dried (Na₂SO₄) and concentrated under reduced pressure. The residue was purified by flash chromatography on silica gel eluted with hexanes/ethyl acetate (1:1) to give methyl N-[(4-bromophenyl)sulfonyl]-L-prolinate (3.98 g, 95%). ¹H NMR (400 MHz CD₃OD) δ 1.75 (m, 1H), 1.95 (m, 2H), 2.06 (m, 1H), 3.30 (m, 1H), 3.48 (m, 1H), 3.72 (s, 3H), 4.27 (dd, 1H), 7.77 (s, 4H); LC-MS m/z 348.0 (MH+), retention time 3.37 minutes.

### Step 2. Preparation of methyl N-[(4′-nitro-1,1′-biphenyl-4-yl)sulfonyl]-L-prolinate

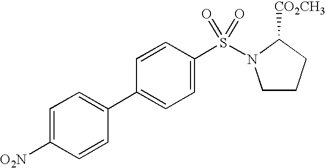

Methyl N-[(4-bromophenyl)sulfonyl]-L-prolinate (1.71 g, 5.0 mmol) and 4-nitrophenyl boronic acid (0.99 g, 6.0 mmol) were combined in a dry flask under argon. Toluene (50 mL), ethanol (17 mL), and a saturated aqueous solution of sodium bicarbonate (17 mL) were added. Argon was bubbled through the mixture for 30 min. Argon flow was maintained while [1,1′-bis(diphenylphosphino)-ferrocene dichloro palladium(II) complex with dichloromethane (1:1) (12 mg, 0.01 mmol) was added. The reaction mixture was heated at 80° C. for 16 h. After cooling to rt, the reaction was diluted with methylene chloride and filtered through Celite®. The organic layer was separated, washed with water and brine, dried (Na₂SO₄), and concentrated under reduced pressure. The residue was purified by flash chromatography (Biotage®) eluted with hexanes/ethyl acetate (3:1) to afford methyl N-[(4′-nitro-1,1′-biphenyl-4-yl)sulfonyl]-L-prolinate (0.65 g, 33%). ¹H NMR (400 MHz, CD₃OD) δ 1.75 (m, 1H), 1.95 (m, 2H), 2.06 (m, 1H), 3.30 (m, 1H), 3.53 (m, 1H), 3.72 (s, 3H), 4.27 (dd, 1H), 7.96 (m, 6H), 8.35 (d, 2H); LC-MS m/z 390.1 (MH+), retention time 3.44 minutes.

### Step 3. Preparation of methyl N-[(4′-amino-1,1′-biphenyl-4-yl)sulfonyl]-L-prolinate

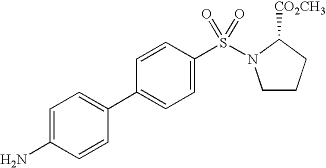

To a solution of methyl N-[(4′-nitro-1,1′-biphenyl-4-yl)sulfonyl]-L-prolinate (0.65 g, 1.7 mmol) in 85% ethanol (40 mL) was added iron powder (0.93 g, 16.7 mmol) and 2N aqueous hydrochloric acid solution (0.84 mL). The resulting mixture was heated at reflux for 2 h. The mixture was filtered through a pad of Celite® and concentrated under reduced pressure. The residue was dissolved in methylene chloride and washed with water and brine. The organic layer was separated, dried (Na₂SO₄), and concentrated under reduced pressure. The residue was purified by flash chromatography (Biotage®) eluted with hexanes/ethyl acetate (5:1) to afford methyl N-[(4′-amino-1,1′-biphenyl-4-yl)sulfonyl]-L-prolinate (0.59 g, 98%). ¹H NMR (400 MHz, CD₃OD) δ 1.75 (m, 1H), 1.95 (m, 2H), 2.06 (m, 1H), 3.30 (m, 1H, 3.53 (m, 1H), 3.72 (s, 3H), 4.27 (dd, 1H), 6.78 (d, 2H), 7.46 (d, 2H), 7.75 (d, 2H), 7.83 (d, 2H); LC-MS m/z 361.1 (MH+), retention time 0.51 minutes.

### Preparation of Methyl N-[(4′-amino-1,1′-biphenyl-4-yl)sulfonyl]-L-valinate

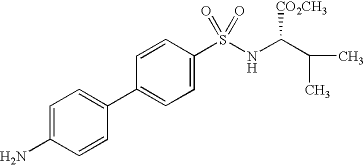

### Step 1. Preparation of methyl N-[(4-bromophenyl)sulfonyl]-L-valinate

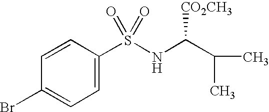

L-valine methyl ester (1.31 g, 7.8 mmol) was suspended in methylene chloride (50 mL) and pyridine (3.2 mL, 39.0 mmol). The resulting mixture was cooled to 0° C., and a solution of bromophenyl sulphonyl chloride (3.0 g, 12.0 mmol) in methylene chloride (20 mL) was added dropwise over a 20-minute period. The cold bath was removed, and the reaction was stirred overnight at rt. The volatile components were removed by rotary evaporation, and the residue was partitioned between methylene chloride and water. The organic layer was separated, washed with 1N aqueous hydrochloric acid solution, water, and brine. The organic layer was dried (Na₂SO₄) and concentrated under reduced pressure. The residue was purified by flash chromatography on silica gel eluted with hexanes/ethyl acetate (1:1) to give methyl N-[(4-bromophenyl)sulfonyl]-L-valinate (2.33 g, 85%).

¹H NMR (400 MHz, CDCl₃) δ 0.88 (d, 3H), 0.97 (d, 3H), 2.06 (m, 1H), 3.49 (s, 3H), 3.74 (dd, 1H), 5.12 (d, 1H), 7.62 (d, 2H), 7.68 (d, 2H); LC-MS m/z 350.0 (MH+), retention time 3.06 minutes.

### Step 2. Preparation of methyl N-[(4′-nitro-1,1′-biphenyl-4-yl)sulfonyl]-L-valinate

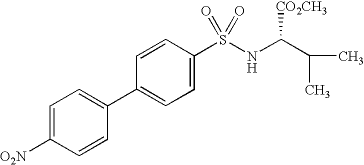

Methyl N-[(4-bromophenyl)sulfonyl]-L-valinate (2.33 g, 6.7 mmol) and 4-nitrophenylboronic acid (1.22 g, 7.3 mmol) were combined in a dry flask under argon. Toluene (50 mL), ethanol (17 mL), and a saturated aqueous solution of sodium bicarbonate (17 mL) were added. Argon was bubbled through the mixture for 30 min. Argon flow was maintained while [1,1′-bis(diphenylphosphino)ferrocene-dichloro palladium(1) complex with dichloromethane (1:1) (27 mg, 0.03 mmol) was added. The reaction mixture was heated at 80° C. for 16 h. After cooling to rt, the reaction was diluted with methylene chloride and filtered through Celite®. The organic layer was separated, washed with water and brine, dried Na₂SO₄), and concentrated under reduced pressure. The residue was purified by flash chromatography (Biotage®) eluted with hexanes/ethyl acetate (3:1) to afford methyl N-[(4′-nitro-1,1′-biphenyl-4-yl)sulfonyl]-L-valinate (1.10 g, 42%). ¹H NMR (400 MHz, CD₃OD) δ 0.93 (d, 3H), 0.95 (d, 3H), 2.03 (m, 1H), 3.66 (s, 3H), 3.72 (dd, 1 Hi), 7.88 (d, 2H), 7.94 (m, 4H), 8.35 (d, 2H); LC-MS m/z 393.1 (MH+), retention time 3.31 minutes.

### Step 3. Preparation of methyl N-[(4′-amino-1,1′-biphenyl-4-yl)sulfonyl]-L-valinate

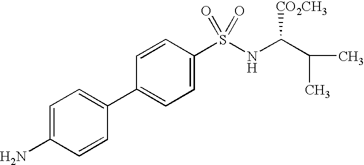

To a solution of methyl N-[(4′-nitro-1,1′-biphenyl-4-yl)sulfonyl]-L-valinate (1.08 g, 2.8 mmol) in 85% ethanol (50 mL) was added iron powder (1.53 g, 27.5 mmol) and 2N aqueous hydrochloric acid solution (1.4 mL). The resulting mixture was heated at reflux for 2 h. The mixture was filtered through a pad of Celite® and concentrated under reduced pressure. The residue was dissolved in methylene chloride and washed with water and brine. The organic layer was separated, dried (Na₂SO₄), and concentrated under reduced pressure. The residue was purified by flash chromatography (Biotage®) eluted with hexanes/ethyl acetate (5:1) to afford methyl N-[(4′-amino-1,1′-biphenyl-4-yl)sulfonyl]-L-valinate (0.92 g, 92%). ¹H NMR (400 MHz, CD₃OD) δ 0.93 (d, 6H, 1.99 (m, 1H), 3.34 (s, 3H), 3.65 (d, 1H), 6.78 (d, 2H), 7.45 (d, 2H), 7.68 (d, 2H), 7.78 (d, 2H); LC-MS m/z 363.1 (MH+), retention time 2.58 minutes.

### Preparation of Methyl N-[(4′-amino-1,1′-biphenyl-4-yl)sulfonyl]-N-methyl-L-valinate

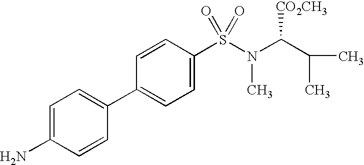

### Step 1. Preparation of methyl N-[(4-bromophenyl)sulfonyl]-N-methyl-L-valinate

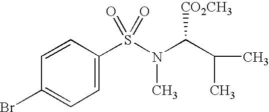

N-methyl L-valine methyl ester (1.78 g, 9.8 mmol) was suspended in methylene chloride (50 mL) and triethylamine (6.82 mL, 48.9 mmol). The mixture was cooled to 0° C., and a solution of 4-bromobenzenesulfonyl chloride (3.0 g, 11.7 mmol) in methylene chloride (20 mL) was added dropwise over 20-minute period. The cold bath was removed, and the reaction was stirred overnight at rt. Volatile components were removed by rotary evaporation, and the residue was partitioned between methylene chloride and water. The organic layer was separated, washed with 1N aqueous hydrochloric acid solution, water, and brine. The organic layer was dried (Na₂SO₄) and concentrated under reduced pressure. The residue was purified by flash chromatography on silica gel eluted with hexanes/ethyl acetate (1:1) to give methyl N-[(4-bromophenyl)sulfonyl]-N-methyl-L-valinate (3.40 g, 95%). ¹H NMR (400 MHz, CD₃OD) δ 0.93 (d, 3H), 0.98 (d, 3H), 2.11 (m, 1H), 2.87 (s, 3H), 3.40 (s, 3H), 4.05 (d, 1H), 7.69 (d, 2H), 7.75 (d, 2H).

### Step 2. Preparation of methyl N-methyl-N-[(4′-nitro-1,1′-biphenyl-4-yl)sulfonyl]-L-valinate

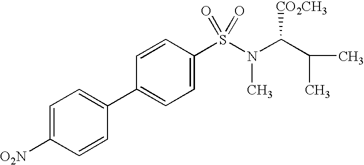

Methyl N-[(4-bromophenyl)sulfonyl]-N-methyl-L-valinate (3.35 g, 9.2 mmol) and 4-nitro-phenylboronic acid (1.69 g, 10.1 mmol) were combined in a dry flask under argon. Toluene (50 mL), ethanol (17 mL), and a saturated aqueous solution of sodium bicarbonate (17 mL) were added. Argon was bubbled through the mixture for 30 min. Argon flow was maintained while [1,1′-bis(diphenyl-phosphino)ferrocene-dichloro palladium(II) complex with dichloromethane (1:1) (37 mg, 0.05 mmol) was added. The reaction mixture was heated at 80° C. for 16 h. After cooling to rt, the reaction was diluted with methylene chloride and filtered through Celite®. The organic layer was separated, washed with water and brine, dried (Na₂SO₄), and concentrated under reduced pressure. The residue was purified by flash chromatography (Biotage®) eluted with hexanes/ethyl acetate (3:1) to afford methyl N-[(4′-nitro-1,1′-biphenyl-4-yl)sulfonyl]-L-valinate (2.21 g, 59%). ¹H NMR (400 MHz, DMSO-d₆) δ 0.86 (d, 3H), 0.89 (d, 3H—), 2.05 (m, 1H), 2.84 (s, 3H), 3.36 (s, 3H), 3.99 (d, 1H), 7.84 (d, 2H), 8.01 (d, 2H), 8.32 (d, 2H).

### Step 3. Preparation of methyl N-[(4′-amino-1,1′-biphenyl-4-yl)sulfonyl]-N-methyl-L-valinate

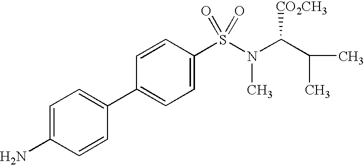

To a solution of methyl N-[(4′-nitro-1,1′-biphenyl-4-yl)sulfonyl]-L-valinate (2.20 g, 5.4 mmol) in 85% ethanol (50 mL) was added iron powder (3.02 g, 54.1 mmol) and 2N aqueous hydrochloric acid solution (2.7 mL). The resulting mixture was heated at reflux for 1 h. The mixture was filtered through a pad of Celite® and concentrated under reduced pressure. The residue was dissolved in methylene chloride and washed with water and brine. The organic layer was separated, dried (Na₂SO₄), and concentrated under reduced pressure. The residue was purified by flash chromatography (Biotage®) eluted with hexanes/ethyl acetate (5:1) to afford methyl N-[(4′-amino-1,1′-biphenyl-4-yl)sulfonyl]-N-methyl-L-valinate (1.40 g, 69%). ¹H NMR (400 MHz, CD₃OD) δ 0.93 (d, 3H), 0.98 (d, 3H), 2.11 (m, 1H), 2.87 (s, 3H), 3.40 (s, 3H), 4.05 (d, 1H), 6.78 (d, 2H), 7.45 (d, 2H), 7.72 (d, 2H), 7.75 (d, 2H).

### Examples of Formula (I)

### N-{[4′-({[(2,4-dichlorophenyl)amino]carbonyl}amino)-1,1′-biphenyl-4-yl]carbonyl}-N methyl-L-valine

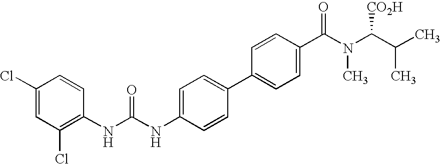

To a solution of N-[(4′-amino-1,1′-biphenyl-4-yl)carbonyl]-N-methyl-L-valinate (50.3 mg, 0.15 mmol) in dichloromethane (2 mL) was added 2,4-dichlorophenyl isocyanate (55.6 mg, 0.30 mmol). The solution was stirred at rt overnight. The mixture concentrated under reduced pressure, and the residue was suspended in ether. The resulting solid was collected by filtration, washed with ether, and dried under high vacuum to give N-{[4′-({[(2,4-dichlorophenyl)amino]-carbonyl}amino)-1,1′-biphenyl-4-yl]carbonyl}-N-methyl-L-valinate (46.0 mg, 59%). ¹H NMR (400 MHz, CD₃OD) δ 0.87 (dd, 3H), 1.09 (dd, 3H), 2.37 (m, 1H), 3.04 (d, 3H), 3.80 (d, 3H), 4.05 (d, 0.5H), 4.80 (d, 0.5H), 7.29 (dd, 1H), 7.46 (s, 1H), 7.48 (d, 1H), 7.61 (m, 4H), 7.72 (d, 2H), 8.18 (d, 1H); LC-MS m/z 528.1 (MH+), retention time 3.80 min.

The intermediate urea (39.0 mg, 0.07 mmol) was dissolved in MeOH (3 mL) and 1N aqueous sodium hydroxide solution (1 mL). The solution was heated at 55° C. overnight. The volatile components were removed by rotary evaporation, and the resulting aqueous mixture was brought to pH 1 with 1N aqueous hydrochloric acid solution. The solid was collected by filtration, washed with water, and dried under vacuum overnight to afford N-{[4′-({[(2,4-dichlorophenyl)amino]carbonyl}-amino-1,1′-biphenyl-4-yl]carbonyl}-N-methyl-L-valine (33.0 mg, 87%). ¹H NMR (400 MHz, CD₃OD) δ 0.89 (dd, 3H), 1.13 (dd, 3H), 2.34 (m, 1H), 3.04 (d, 31H), 4.05 (d, 0.5H), 4.80 (d, 0.5H), 7.29 (dd, 1H), 7.55 (m, 7H), 7.72 (d, 2H), 8.18 (d, 1H); LC-MS m/z 514.1 (MH+), retention time 3.47 min.

### N-({4′-[(3,4-dichlorobenzoyl)amino]-1,1′-biphenyl-4-yl}carbonyl)-N-methyl-L-valine

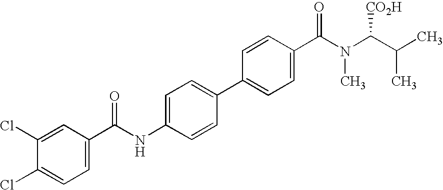

To a solution of N-[(4′-amino-1,1′-biphenyl-4-yl)carbonyl]-N-methyl-L-valinate (50 mg, 0.15 mmol) in dichloromethane (2 mL) was added 3,4-dichlorobenzoyl chloride (62 mg, 0.30 mmol) and triethylamine (46 mg, 0.45 mmol). The solution was stirred at rt overnight. The solution concentrated under reduced pressure, and the residue was suspended in ether. The resulting solid was collected by filtration, washed with ether, and dried under high vacuum to give N-({4′-[(3,4-dichlorobenzoyl)amino]-1,1′-biphenyl-4-yl}carbonyl)-N-methyl-L-valinate (60 mg, 79%). ¹H NMR (400 MHz, CD₃OD) δ 0.88 (dd, 3H), 1.09 (dd, 3H), 2.36 (m, 1H), 3.04 (d, 3H), 3.79 (d, 3H), 4.03 (d, 0.5H), 4.80 (d, 0.5H), 7.49 (d, 2H), 7.71 (m, 3H), 7.75 (d, 2H), 7.83 (d, 2H), 7.88 (dd, 1H), 8.14 (d, 1H); LC-MS m/z 513.1 (MH+), retention time 3.70 min.

The intermediate amide (60 mg, 0.12 mmol) was dissolved in MeOH (3 mL) and 1N aqueous sodium hydroxide solution (1 mL). The solution was heated at 55° C. overnight. The volatile components were removed by rotary evaporation, and the resulting aqueous mixture was brought to pH 1 with 1N aqueous hydrochloric acid solution. The solid was collected by filtration, washed with water, and dried under vacuum overnight to afford N-({4′-[(3,4-dichlorobenzoyl)amino]-1,1′-biphenyl-4-yl}carbonyl)-N-methyl-L-valine (30 mg, 50%). ¹H NMR (400 MHz, CD₃OD) δ 0.88 (d, 3H), 1.12 (dd, 3H), 2.34 (m, 1H), 3.06 (d, 3H), 3.99 (d, 0.5H), 4.82 (d, 0.5H), 7.51 (t, 2H), 7.69 (m, 3H), 7.75 (d, 2H), 7.83 (d, 2H), 7.88 (dd, 1H), 8.14 (d, 1H); LC-MS m/z 499.1 (MH+), retention time 3.42 mL

### N-[(4′-({[(4-ethoxyphenyl)acetyl]amino}-1,1′-biphenyl-4-yl)carbonyl]-N-methyl-L-valine

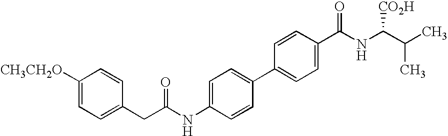

To a solution of methyl N-[(4′-amino-1,1′-biphenyl-4-yl)carbonyl]-L-valinate (70 mg, 0.21 mmol) in dichloromethane (3 mL) was added 4-ethoxyphenylacetic acid (50 mg, 0.28 mmol), dimethylaminopyridine (13 mg, 0.11 mmol), and 1-(3-dimethylaminopropyl)-3-ethylcarbodiimide hydrochloride (53 mg, 0.28 mmol). The solution was stirred at rt for 48 h then concentrated under reduced pressure. The residue was suspended in ether and filtered. The resulting solid was washed with ether, 1N aqueous hydrochloric acid solution, and dried under high vacuum to afford methyl N-[(4′-({[(4-ethoxyphenyl)acetyl]amino}-1,1′-biphenyl-4-yl)carbonyl]-N-methyl-L-valinate (68 mg, 66%). ¹H NMR (400 MHz, CD₃OD) δ 1.06 (dd, 6H), 138 (t, 3H), 2.26 (m, 1H), 3.63 (s, 3H), 3.76 (s, 3H), 4.02 (q, 2H), 4.50 (d, 1H), 6.87 (d, 2H), 7.25 (d, 2H), 7.66 (m, 4H), 7.70 (d, 2H), 7.89 (d, 2H); LC-MS m/z 489.2 (MH+), retention time 3.11 min.

The intermediate amide (65 mg, 0.13 mmol) was dissolved in MeOH (3 mL) and 1N aqueous sodium hydroxide solution (1 mL). The solution was heated at 55° C. overnight. The volatile components were removed by rotary evaporation, and the resulting aqueous mixture was brought to pH 1 with 1N aqueous hydrochloric acid solution. The solid was collected by filtration, washed with water, and dried under vacuum overnight to afford N-[(4′-({[(4-ethoxyphenyl)acetyl]amino}-1,1′-biphenyl-4-yl)carbonyl]-N-methyl-L-valine (57 mg, 90%). ¹H NMR (400 MHz, CD₃OD) δ 1.06 (dd, 6H), 1.38 (t, 3H), 2.29 (m, 1H), 3.63 (s, 3H), 4.02 (q, 2H), 4.50 (d, 1H), 6.87 (d, 2H), 7.25 (d, 2H), 7.66 (m, 6H), 7.91 (d, 2H); LC-MS m/z 475.2 (MH+), retention time 3.03 min.

### N-{[4′-({[(2,4-dimethylphenyl)amino]carbonyl}amino)biphenyl-4-yl]carbonyl}-2-methylalanine

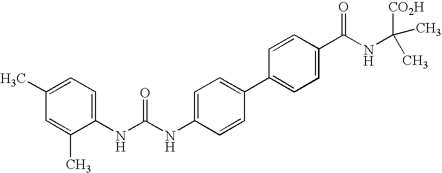

To a solution of methyl N-[(4′-aminobiphenyl-4-yl)carbonyl]-2-methylalaninate (30.0 mg, 0.10 mmol) in dichloroethane (4 mL) was added 2,4-dimethylphenyl isocyanate (21.2 mg, 0.14 mmol). The solution was stirred at rt overnight. The mixture was evaporated to dryness under reduced pressure, and the crude residue was used in the next step without purification. The intermediate urea was dissolved in methanol (0.8 mL) and tetrahydrofuran (0.8 mL). Aqueous sodium hydroxide solution (1N, 0.12 mL, 0.12 mmol) was added, and the solution was stirred at rt overnight. The reaction mixture was filtered, and the filtrate was purified by preparative reverse-phase HPLC (water/acetonitrile gradient, containing 0.1% TFA) to give N-{[4′-({[(2,4-di-methylphenyl)amino]carbonyl}amino)biphenyl-4-yl]carbonyl}-2-methylalanine (15.3 mg, 34%). ¹H NMR (400 MHz, DMSO-d₆) δ 1.48 (s, 6H), 2.21 (s, 3H), 2.23 (s, 3H), 6.94 (d, 1H), 6.99 (s, 1H), 7.55 (d, 2H), 7.64-7.68 (m, 3H), 7.70 (d, 2H), 7.87-7.92 (m, 3H), 8.43 (s, 1H), 9.08 (s, 1H), 12.12 (s, 1H); LC-MS m/z 446.1 (MH+), retention time 3.03 min.

### N-[(4′-({[(3-fluoro-4-methylphenyl)amino]carbonyl}biphenyl-4-yl)carbonyl]-N,2-dimethylalanine

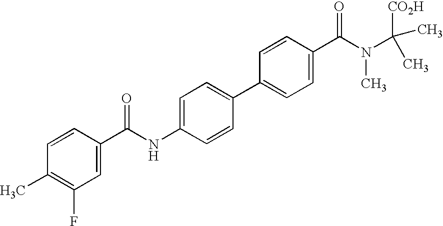

To a solution of methyl N-[(4′-aminobiphenyl-4-yl)carbonyl]-N,2-dimethylalaninate (35.0 mg, 0.11 mmol) in dichloroethane (4 mL) was added 3-fluoro-4-methylbenzoyl chloride (22.2 mg, 0.13 mmol) and polymer-supported diisopropylethylamine (56.0 mg, 0.21 mmol). The resulting mixture was stirred at rt overnight The solids were removed by filtration, and the filtrate was evaporated to dryness under reduced pressure. The crude methyl N-({4′-[(3-fluoromethyl-benzoyl)amino]biphenyl-4-yl}carbonyl)-N,2-dimethylalaninate was used in the next step without further purification.

The intermediate amide (46.3 mg, 0.10 mmol) was dissolved in methanol (0.8 mL) and tetrahydrofuran (0.8 mL). Aqueous sodium hydroxide solution (1N, 0.2 mL, 0.20 mmol) was added, and the solution was stirred at rt overnight. Additional aqueous potassium hydroxide solution (3N, 0.20 mL, 0.60 mmol) was added, and the reaction mixture was heated at 65° C. for two days. The reaction mixture was filtered, and the filtrate was purified by preparative reverse-phase HPLC (water/acetonitrile gradient, containing 0.1% TFA) to give N-({4′-[(3-fluoro-4-methylbenzoyl)-amino]biphenyl-4-yl}carbonyl)-N,2 dimethylalanine (2.3 mg, 5%). ¹H NMR (400 MHz, DMSO-d₆) δ 1.23 (s, 6H), 2.32 (s, 3H), 2.93 (s, 3H), 7.42-7.48 (m, 3H), 7.70-7.79 (m, 6H), 7.87 (d, 2H), 10.34 (s, 1H), 12.03 (s, 1H); LC-MS m/z 449.0 (MH+), retention time 3.00 min.

### N-[(4-{[(5-chloro-2,3-dihydro-1H-indol-1-yl)carbonyl]amino}1,1′-biphenyl-4-yl)carbonyl]-L-valine

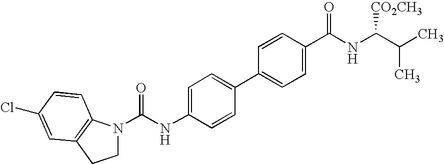

Under an argon atmosphere, methyl N-[(4′-amino-1,1′-biphenyl-4-yl)carbonyl]-L-valinate (0.10 g, 0.11 mmol) was suspended in toluene (3 mL) and triethylamine (1.00 mL, 7.17 mmol). The mixture was cooled to 0° C. and vented to 2N aqueous sodium hydroxide solution. Phosgene (20% in toluene, 1.60 mL, 3.06 mmol) was slowly introduced. The mixture was allowed to warm to rt then was stirred for an additional 2 h. The resulting suspension was filtered, and the filtrate was concentrated under reduced pressure. A dark orange oil was obtained and dissolved in 1,2-dichloro-ethane (6 mL). 5-Chloro-2,3-dihydro-(1H)-indole (0.05 g, 0.46 mmol) was added. The mixture was stirred at rt overnight then concentrated under reduced pressure. The residue was suspended in ethyl acetate, and the resulting solid was collected by filtration. The crude solid was purified by flash chromatography on silica gel eluted with hexanes/ethyl acetate (2:1) to provide methyl N-[(4′-({[(5-chloro-2,3-dihydro-1H-indol-1-yl)carbonyl]amino}-1,1′-biphenyl-4-yl)carbonyl]-L-valinate (40 mg, 25%). ¹H NMR (400 MHz, CD₂Cl₂) δ 1.04 (t, 6H), 2.29 (m, 1H), 3.28 (t, 2H), 3.79 (s, 3H), 4.14 (t, 2H), 4.74 (m, 1H), 6.60 (m, 1H), 7.17 (m, 2H), 7.58 (d, 2H), 7.64 (d, 2H), 7.70 (d, 2H), 7.87 (d, 2H), 7.93 (d, 1H); LC-MS m/z 506.2 (MH+), retention time 3.63 min.

The intermediate urea (36 mg, 0.07 mmol) was dissolved in methanol (3 mL) and 1N aqueous sodium hydroxide solution (1 mL). The solution was heated at 75° C. for 2 h, then concentrated under reduced pressure to remove volatile components. The aqueous mixture was brought to pH 2 with the addition of 1N aqueous hydrochloric acid solution. The resulting solid was collected by filtration, washed with water, and dried under vacuum overnight to provide N-[(4′-({[(5-chloro-2,3-dihydro-1H-indol-1-yl)carbonyl]amino}-1,1′-biphenyl-4-yl) carbonyl]-L-valine (17 mg, 50%). ¹H NMR (400 MHz, CD₃OD) δ 1.04 (d, 6H), 2.31 (m, 1H), 3.26 (t, 2H), 4.19 (t, 2H), 4.53 (m, 1H), 7.11 (d, 1H), 7.19 (s, 1H), 7.58 (d, 2H), 7.64 (d, 2H), 7.73 (d, 2H), 7.85 (d, 2H), 7.93 (d, 1H); LC-MS m/z/z 492.1 (MH+), retention time 3.36 min.

### N-[(4′-({[(7-methoxy-1-benzofuran-2-yl)carbonyl]amino}-1,1′-biphenyl-4-yl)carbonyl]-L-valine

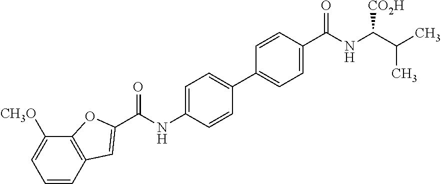

To a solution of methyl N-[(4′-amino-1,1′-biphenyl-4-yl)carbonyl]-L-valinate (70 mg, 0.21 mmol) in dichloromethane (3 mL) was added benzofuran-2-carboxylic acid (45 mg, 0.28 mmol), 4-dimethylaminopyridine (13 mg, 0.11 mmol), and 1-(3-dimethylaminopropyl)-3-ethylcarbodiimide hydrochloride (53 mg, 0.28 mmol). The solution was heated at 55° C. for 18 h and concentrated under reduced pressure to dryness. The residue was suspended in ether, and the solid was collected by filtration. The solid was washed with ether, 1N aqueous hydrochloric acid solution, and dried under vacuum to afford methyl N-[(4′-({[(7-methoxy-1-benzofuran-2-yl)carbonyl]amino}-1,1′-biphenyl-4-yl)carbonyl]-L-valinate (66 mg, 59%). ¹H NMR (400 MHz, CD₃OD) δ 1.09 (d, 6H), 2.31 (m, 1H), 3.77 (s, 3H), 4.06 (s, 3H), 4.52 (m, 1H), 7.07 (d, 1H), 7.28 (m, 2H), 7.62 (s, 1H), 7.72 (d, 2H), 7.76 (d, 2H), 7.89 (d, 2H), 7.93 (d, 2H); LC-MS m/z 501.2 (MH+), retention time 3.37 minutes.

The intermediate amide (58 mg, 0.12 mmol) was dissolved in methanol (3 mL) and 1N aqueous sodium hydroxide solution (1 mL). The mixture was heated at 55° C. overnight, then the volatile components were removed under reduced pressure. The resulting suspension was brought to pH 2 by addition of 1N aqueous hydrochloric acid solution. The solid was collected by filtration, washed with water, and dried under vacuum overnight to afford N-[(4′-({[(4-ethoxyphenyl)acetyl]-amino}-1,1′-biphenyl-4-yl)carbonyl]-N-methyl-L-valine (47 mg, 83%). ¹H NMR (400 MHz, CD₃OD) δ 1.09 (d, 6H), 2.31 (m, 1H), 4.06 (s, 3H), 4.52 (m, 1H), 7.07 (d, 1H), 7.28 (m, 2H), 7.62 (s, 1H), 7.72 (d, 2H), 7.76 (d, 2H), 7.89 (d, 2H), 7.93 (d, 2H); LC-MS m/z 487.2 (MH+), retention time 3.18 min.

### N-{[4′-({[(2-Chlorophenyl)amino]carbonyl}amino)-1,1′-biphenyl-4-yl]sulfonyl}-L-proline

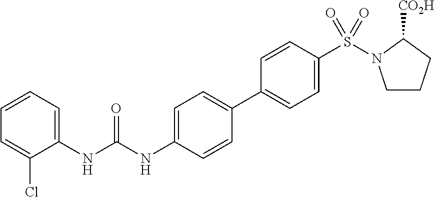

To a solution of methyl N-[(4′-amino-1,1′-biphenyl-4-yl)sulfonyl]-L-prolinate (40 mg, 0.11 mmol) in dichloromethane (2 mL) was added 2-chlorophenyl isocyanate (35 mg, 0.23 mmol). The solution was stirred at rt overnight, then concentrated to dryness under reduced pressure. The residue was suspended in ether, and the solid was collected by filtration, washed with fresh ether, and dried under vacuum to give methyl N-{[4′-({[(2-chlorophenyl)amino]carbonyl}amino)-1,1′-biphenyl-4-yl]sulfonyl}-L-prolinate (46.0 mg, 59%). ¹H NMR (400 MHz, CD₂Cl) δ 1.75 (m, 1H), 2.02 (m, 3H), 3.30 (m, 1H), 3.53 (m, 1H), 3.72 (s, 3H), 4.27 (m, 1H), 7.06 (m, 3H), 7.29 (t, 1H), 7.46 (d, 1H), 7.55 (d, 2H), 7.62 (d, 2H), 7.74 (d, 2H), 7.88 (d, 2H), 8.18 (d, 1H); LC-MS m/z 514.1 (MH+), retention time 3.62 min.

The intermediate urea (36 mg, 0.07 mmol) was dissolved in methanol (1 mL) and 1N aqueous sodium hydroxide solution (0.5 mL). The mixture was heated at 55° C. overnight, then the volatile components were removed under reduced pressure. The resulting suspension was brought to pH 1 by addition of 1N aqueous hydrochloric acid solution. The solid was collected by filtration, washed with water, and dried under vacuum overnight to afford N-{[4′-({[(2-chlorophenyl)amino]carbonyl}-amino)-1,1′-biphenyl-4-yl]sulfonyl}-L-proline (29 mg, 82%). ¹H NMR (400 MHz, CD₃OD) δ 1.75 (m, 1H), 1.99 (m, 3H), 3.30 (m, 1H), 3.51 (m, 1H), 4.25 (m, 1H), 7.03 (ddd, 1H), 7.29 (ddd, 1H), 7.41 (ddd, 1H), 7.61 (d, 2H), 7.68 (d, 2H), 7.84 (d, 2H), 7.92 (d, 2H), 8.14 (d, 2H); LC-MS m/z 500.1 (MH+), retention time 3.40 min.

### N-({4′-[(3,4-difluorobenzoyl)amino]-1,1′-biphenyl-4-yl}sulfonyl)-L-valine

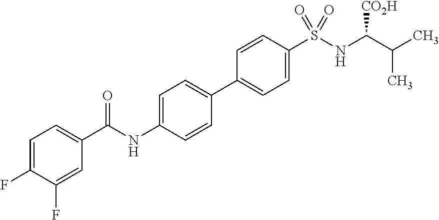

To a solution of methyl N-[(4′-amino-1,1′-biphenyl-4-yl)sulfonyl]-L-valinate (44 mg, 0.12 mmol) in dichloromethane (2 mL) was added 3,4-difluorobenzoyl chloride (43 mg, 0.25 mmol) and pyridine (29 mg, 0.37 mmol). The solution was stirred at rt overnight. The mixture was evaporated to dryness under reduced pressure, and the residue was suspended in ether. The solid was collected by filtration, washed with fresh ether, and dried under vacuum. The dried material was dissolved in methanol (3 mL) and 1N aqueous sodium hydroxide solution (1 mL). The mixture was heated at 55° C. overnight, then the volatile components were removed under reduced pressure. The resulting suspension was brought to pH 1 with 1N aqueous hydrochloric acid solution. The solid was collected by filtration, washed with water, and dried under vacuum to afford N-({4′-[(3,4-difluorobenzoyl)amino]-1,1′-biphenyl-4-yl}sulfonyl)-L-valine (24 mg, 45%). ¹H NMR (400 MHz, CD₃OD) δ 0.93 (d, 6H), 0.99 (d, 3H), 2.06 (m, 1H), 3.65 (d, 1H), 7.22 (t, 1H), 7.43 (dd, 1H), 7.71 (m, 2H), 7.81 (m, 5H), 7.89 (d, 2H); LC-MS m/z 489.1 (MH+), retention time 3.16 min.

### N-({4′-[(3,4-dimethylbenzoyl)amino]-1,1′-biphenyl-4-yl}sulfonyl)-N-methyl-L-valine

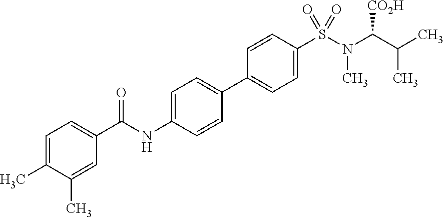

To a solution of methyl N-[(4′-amino-1,1′-biphenyl-4-yl)sulfonyl]-N-methyl-L-valinate (60 mg, 0.16 mmol) in dichloromethane (3 mL) was added 3,4-dimethylbenzoyl chloride (54 mg, 0.32 mmol) and triethylamine (48 mg, 0.48 mmol). The solution was stirred at rt overnight. The mixture was evaporated to dryness under reduced pressure, and the residue was suspended in ether. The solid was collected by filtration, washed with fresh ether, and dried under vacuum to afford methyl N-({4′-[(3,4-dimethylbenzoyl)amino]-1,1′-biphenyl-4-yl}sulfonyl)-N-methyl-L-valinate (54 mg, 66%). ¹H NMR (400 MHz, CD₃OD) δ 0.93 (d, 3H), 1.01 (d, 3H), 2.11 (m, 1H), 2.37 (d, 6H), 2.92 (s, 3H), 3.40 (s, 3H), 4.05 (d, 1H), 7.25 (d, 2H), 7.72 (m, 4H), 7.84 (m, 6H); LC-MS m/z 509.2 (MH+), retention time 3.77 min.

The intermediate amide (48 mg, 0.09 mmol) was dissolved in methanol (3 i) and 1N aqueous sodium hydroxide solution (1 mL). The mixture was heated at 75° C. for 2 h, then the volatile components were removed under reduced pressure. The resulting suspension was brought to pH 1 by addition of 1N aqueous hydrochloric acid solution. The solid was collected by filtration, washed with water, and dried under vacuum to afford N-({-4′-[(3,4-dimethylbenzoyl)amino]-1,1′-biphenyl-4-yl}sulfonyl)-N-methyl-L-valine (35 mg, 87%). ¹H NMR (400 MHz, CD₃OD) δ 0.99 (d, 3H), 1.05 (d, 3 M), 2.11 (m, 1H), 2.37 (d, 6H), 2.92 (s, 3H), 4.05 (d, 1H), 7.78 (d, 1H), 7.69 (m, 4H), 7.84 (m, 7H); LC-MS m/z 495.2 (MH+), retention time 3.51 min.

### N-[(4′-({[(3,5-difluorophenyl)acetyl]amino}-1,1′-biphenyl-4-yl)sulfonyl]-N-methyl-L-valine

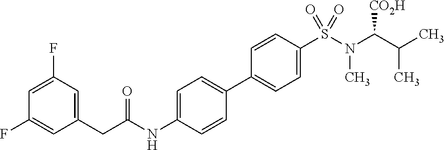

To a solution of methyl N-[(4′-amino-1,1′-biphenyl-4-yl)sulfonyl]-N-methyl-L-valinate (80 mg, 0.21 mmol) in dichloromethane (3 mL) was added 3,5-difluorophenylacetic acid (73 mg, 0.42 mmol), 4-dimethylaminopyridine (52 mg, 0.42 mmol), and 1-(3-dimethylaminopropyl)-3-ethylcarbodiimide hydrochloride (81 mg, 0.42 mmol). The mixture was heated at 55° C. for 18 h. The mixture was allowed to cool to ambient temperature and was diluted with methylene chloride. The organic mixture was washed with 1N aqueous hydrochloric acid solution and brine then concentrated to dryness under reduced pressure. The residue was suspended in ether, and the solid was collected by filtration. The solid was washed with ether and dried under high vacuum to afford methyl N-[(4′-({[(3,5-difluorophenyl)acetyl]amino}-1,1′-biphenyl-4-yl)sulfonyl]-N-methyl-L-valinate (65 mg, 58%). ¹H NMR (400 MHz, CD₃OD) δ 0.90 (d, 3H), 0.97 (d, 3H), 2.08 (m, 1H), 2.88 (s, 3H, 3.35 (s, 3H), 3.72 (s, 2H), 4.05 (d, 1H), 6.82 (t, 1H), 6.96 (d, 2H), 7.61 (d, 2H), 7.68 (d, 2H), 7.74 (d, 2H), 7.78 (d, 2H); LC-MS m/z 531.2 (MH+), retention time 3.62 min.

The intermediate benzyl amide (65 mg, 0.12 mmol) was dissolved in methanol (3 mL) and 1N aqueous sodium hydroxide solution (1 mL). The mixture was heated at 75° C. for 2 h, then the volatile components were removed under reduced pressure. The resulting suspension was brought to pH 1 with 1N aqueous hydrochloric acid solution. The solid was collected by filtration, washed with water, and dried under vacuum to afford N-[(4′-({[(3,5-difluorophenyl)acetyl]amino}-1,1′-biphenyl-4-yl)sulfonyl]-N-methyl-L-valinate (57 mg, 90%). ¹H NMR (400 M CD₃OD) δ 0.99 (t, 6H), 2.08 (m, 1H), 2.90 (s, 3H), 3.72 (s, 2H), 4.05 (d, 1H), 6.86 (t, 1H), 6.99 (d, 2H), 7.66 (d, 2H), 7.68 (d, 2H), 7.77 (d, 2H), 7.86 (d, 2H); LC-MS m/z 517.2 (MH+), retention time 3.31 min.

By using the methods described above and by selecting the appropriate starting materials, other compounds of the invention were prepared and characterized. These compounds, together with the Examples described above, are summarized in Tables 1 and 2.

TABLE 1

| Example | | LC-MS | LC-MS ret. | LC-MS | chiral- | |
|---|---|---|---|---|---|---|
| No. | structure | MH+ (m/z) | time (min) | method | ity | IUPAC name |
| 1 | 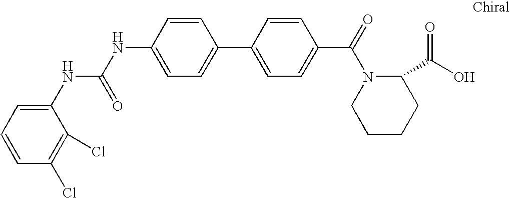 | 511.1 | 3.47 | 2 | S | (2S)-1-{[4′-({[(2,3-dichlorophenyl)- amino]carbonly}amino)-1,1′-biphenyl-4- yl]carbonyl}-2-piperidinecarboxylic acid |
| 2 | 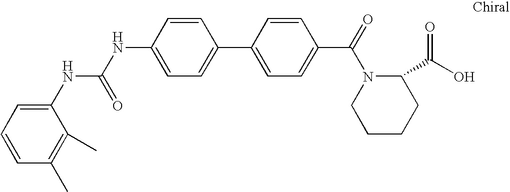 | 472.2 | 3.16 | 2 | S | (2S)-1-{[4′-({[(2,3-dimethylphenyl)- amino]carbonyl}amino)-1,1′-biphenyl-4- yl]carbonyl}-2-piperidinecarboxylic acid |
| 3 | 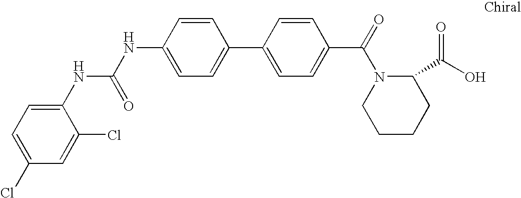 | 512.1 | 3.52 | 2 | S | (2S)-1-{[4′-({[(2,4-dichlorophenyl)- amino]carbonyl}amino)-1,1′-biphenyl-4- yl]carbonyl}-2-piperidinecarboxylic acid |
| 4 | 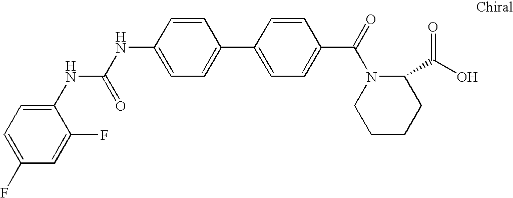 | 480.2 | 3.03 | 2 | S | (2S)-1-{[4′-({[(2,4-difluorophenyl)- amino]carbonyl}amino)-1,1′-biphenyl-4- yl]carbonyl}-2-piperidinecarboxylic acid |
| 5 | 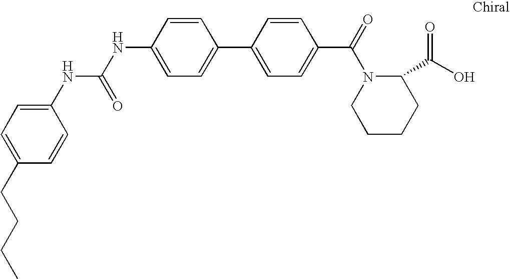 | 500.2 | 3.53 | 2 | S | (2S)-1-{[4′-({[(4-butylphenyl)- amino]carbonyl}amino)-1,1′-biphenyl-4- yl]carbonyl}-2-piperidinecarboxylic acid |
| 6 | 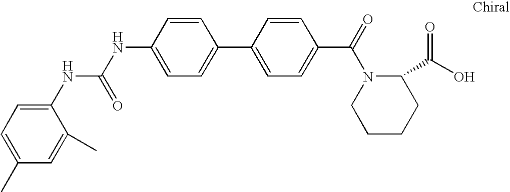 | 472.2 | 3.21 | 2 | S | (2S)-1-{[4′-({[(2,4-dimethylphenyl)- amino]carbonyl}amino)-1,1′-biphenyl-4- yl]carbonyl}-2-piperidinecarboxylic acid |
| 7 | 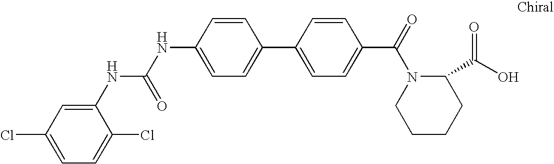 | 512.1 | 3.44 | 2 | S | (2S)-1-{[4′-({[(2,5-dichlorophenyl)- amino]carbonyl}amino)-1,1′-biphenyl-4- yl]carbonyl}-2-piperidinecarboxylic acid |
| 8 | 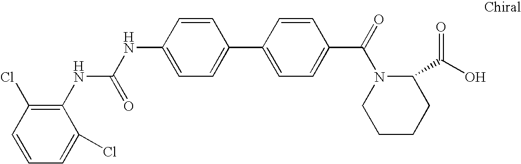 | 512.1 | 3.00 | 2 | S | (2S)-1-{[4′-({[(2,6-dichlorophenyl)- amino]carbonyl}amino)-1,1′-biphenyl-4- yl]carbonyl}-2-piperidinecarboxylic acid |
| 9 | 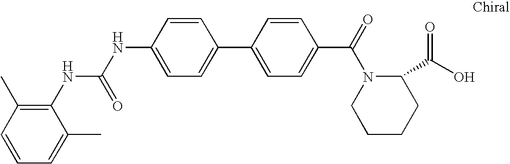 | 472.2 | 2.85 | 2 | S | (2S)-1-{[4′-({[(2,6-dimethylphenyl)- amino]carbonyl}amino)-1,1′-biphenyl-4- yl]carbonyl}-2-piperidinecarboxylic acid |
| 10 | 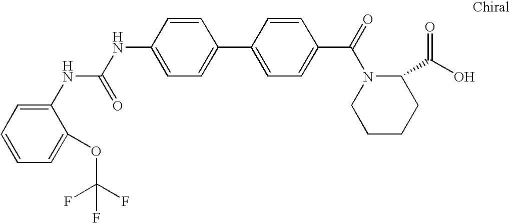 | 528.2 | 3.38 | 2 | S | (2S)-1-{[4′-({[(2-trifluoromethoxyphenyl)- amino]carbonyl}amino)-1,1′-bipheny(-4- yl]carbonyl}-2-piperidinecarboxylic acid |
| 11 | 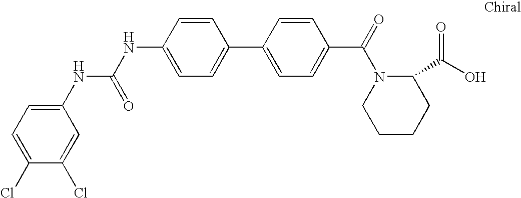 | 512.2 | 3.40 | 2 | S | (2S)-1-{[4′-({[(3,4-dichlorophenyl)- amino]carbonyl}amino)-1,1′-biphenyl-4- yl]carbonyl}-2-piperidinecarboxylic acid |
| 12 | 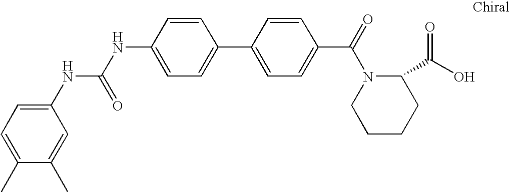 | 472.2 | 3.20 | 2 | S | (2S)-1-{[4′-({[(3,4-dimethylphenyl)- amino]carbonyl}amino)-1,1′-biphenyl-4- yl]carbonyl}-2-piperidinecarboxylic acid |
| 13 | 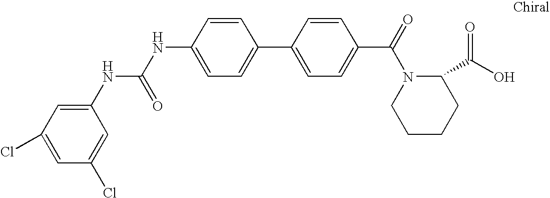 | 512.1 | 3.55 | 2 | S | (2S)-1-{[4′-({[(3,5-dichlorophenyl)- amino]carbonyl}amino)-1,1′-biphenyl-4- yl]carbonyl}-2-piperidinecarboxylic acid |
| 14 | 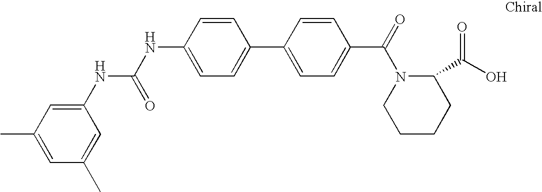 | 472.2 | 3.29 | 2 | S | (2S)-1-{[4′-({[(3,5-dimethylphenyl)- amino]carbonyl}amino)-1,1′-biphenyl-4- yl]carbonyl}-2-piperidinecarboxylic acid |
| 15 | 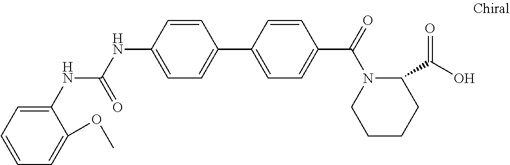 | 474.2 | 3.15 | 2 | S | (2S)-1-{[4′-({[(2-methoxyphenyl)- amino]carbonyl}amino)-1,1′-biphenyl-4- yl]carbonyl}-2-piperidinecarboxylic acid |
| 16 | 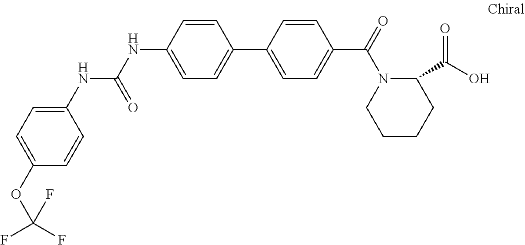 | 528.2 | 3.30 | 2 | S | (2S)-1-{[4′-({[(4-trifluoromethoxyphenyl)- amino]carbonyl}amino)-1,1′-biphenyl-4- yl]carbonyl}-2-piperidinecarboxylic acid |
| 17 | 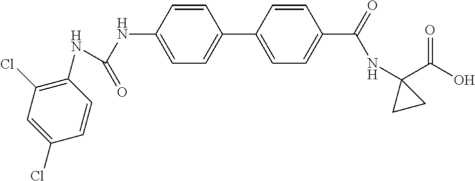 | 484.1 | 3.10 | 1 | not chiral | 1-({[4′-({[(2,4-dichlorophenyl)- amino]carbonyl}amino)-1,1′-biphenyl-4- yl]carbonyl}amino)cyclopropanecarboxylic acid |
| 18 | 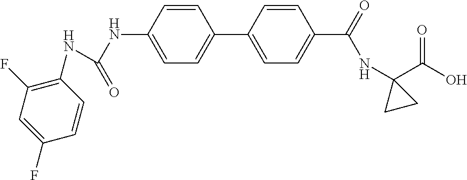 | 452.2 | 2.78 | 1 | not chiral | 1-({[4′-({[(2,4-difluorophenyl)- amino]carbonyl}amino)-1,1′-biphenyl-4- yl]carbonyl}amino)cyclopropanecarboxylic acid |
| 19 | 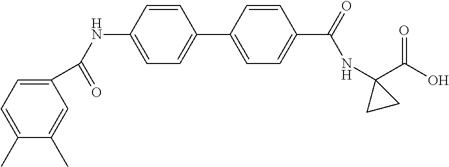 | 429.2 | 2.94 | 2 | not chiral | 1-({[4′-({[(3,4-dimethylphenyl)- amino]carbonyl}amino)-1,1′-biphenyl-4- yl]carbonyl}amino)cyclopropanecarboxylic acid |
| 20 | 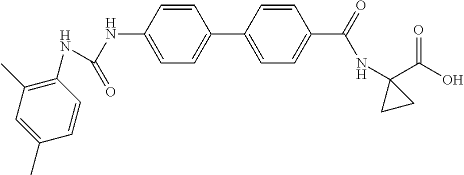 | 444.2 | 2.96 | 2 | not chiral | 1-({[4′-({[(2,4-dimethylphenyl)- amino]carbonyl}amino)-1,1′-biphenyl-4- yl]carbonyl}amino)cyclopropanecarboxylic acid |
| 21 | 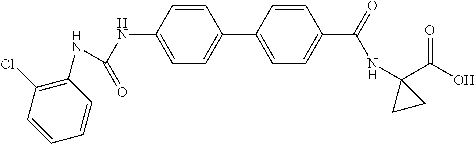 | 450.1 | 2.82 | 1 | not chiral | 1-({[4′-({[(2-chlorophenyl)- amino]carbonyl}amino)-1,1′-biphenyl-4- yl]carbonyl}amino)cyclopropanecarboxylic acid |
| 22 | 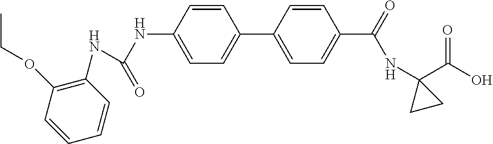 | 460.2 | 2.58 | 2 | not chiral | 1-({[4′-({[(2-ethoxyphenyl)- amino]carbonyl}amino)-1,1′-biphenyl-4- yl]carbonyl}amino)cyclopropanecarboxylic acid |
| 23 | 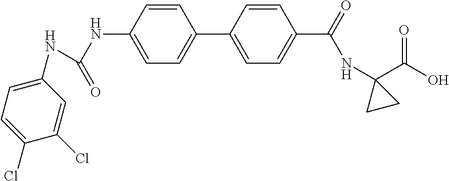 | 484.1 | 3.07 | 1 | not chiral | 1-({[4′-({[(3,4-dichlorophenyl)- amino]carbonyl}amino)-1,1′-biphenyl-4- yl]carbonyl}amino)cyclopropanecarboxylic acid |
| 24 | 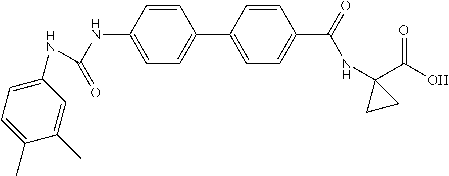 | 444.2 | 2.88 | 1 | not chiral | 1-({[4′-({[(3,4-dimethylphenyl)- amino]carbonyl}amino)-1,1′-biphenyl-4- yl]carbonyl}amino)cyclopropanecarboxylic acid |
| 25 | 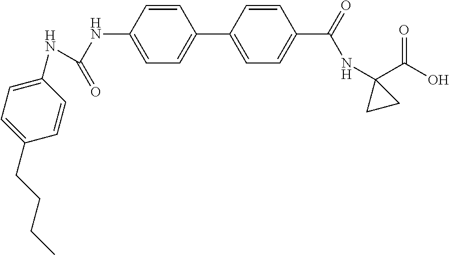 | 472.0 | 3.23 | 1 | not chiral | 1-({[4′-({[(2,4-butylphenyl)- amino]carbonyl}amino)-1,1′-biphenyl-4- yl]carbonyl}amino)cyclopropanecarboxylic acid |
| 26 | 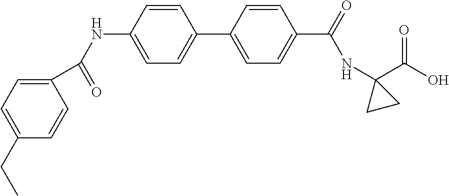 | 429.2 | 3.03 | 2 | not chiral | 1-({[4′-({[(4-ethylphenyl)- amino]carbonyl}amino)-1,1′-biphenyl-4- yl]carbonyl}amino)cyclopropanecarboxylic acid |
| 27 | 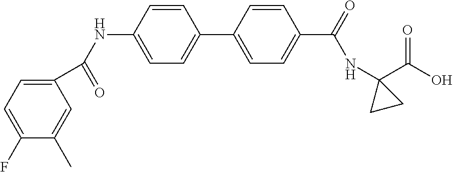 | 433.2 | 3.31 | 1 | not chiral | 1-({[4′-({[(4-fluoro-3-methylphenyl)- amino]carbonyl}amino)-1,1′-biphenyl-4- yl]carbonyl}amino)cyclopropanecarboxylic acid |
| 28 | 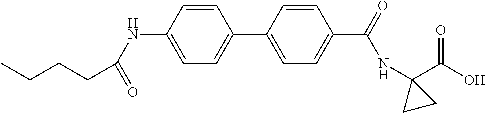 | 381.2 | 3.02 | 1 | not chiral | 1-({[4′-(pentanoylamino)biphenyl-4- yl]carbonyl]amino]carbonyl}amino)cyclopropane- carboxylic acid |
| 29 | 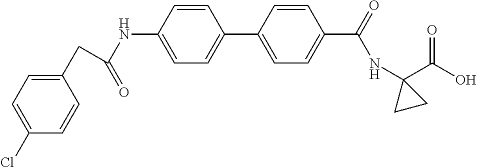 | 449.1 | 2.88 | 2 | not chiral | 1-[({4′-[((4-chlorophenyl)-acetyl)amino]- 1,1′-biphenyl-4- yl}carbonyl)amino]cyclopropanecarboxylic acid |
| 30 | 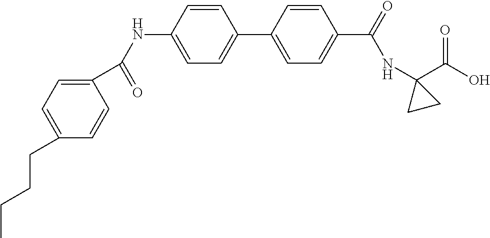 | 457.2 | 3.27 | 2 | not chiral | 1-[({4′-[(4-butylbenzoyl)amino]-1,1′- biphenyl-4- yl}carbonyl)amino]cyclopropanecarboxylic acid |
| 31 | 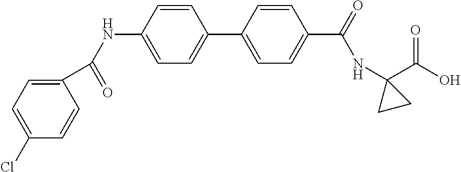 | 434.1 | 2.80 | 1 | not chiral | 1-[({4′-[(4-chlorobenzoyl)amino]-1,1′- biphenyl-4- yl}carbonyl)amino]cyclopropanecarboxylic acid |
| 32 | 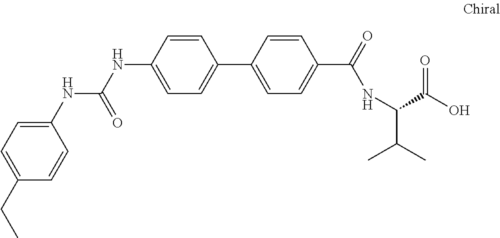 | 460.2 | 3.15 | 2 | S | N-{[4′-({[4-ethylphenyl)-amino]carbonyl}amino)biphenyl-4- yl]carbonyl}-L-valine |
| 33 | 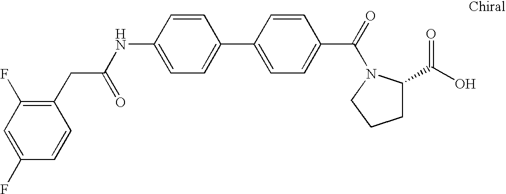 | 465.2 | 3.00 | 2 | S | 1-[(4′-{[(2,4-difluorophenyl)-acetyl]amino}- 1,1′-biphenyl-4-yl)carbonyl]-L-proline |
| 34 | 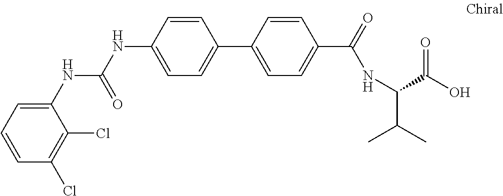 | 500.1 | 3.35 | 2 | S | N-{[4′-({[2,3-dichlorophenyl)-amino]carbonyl}amino)biphenyl-4- yl]carbonyl}-L-valine |
| 35 | 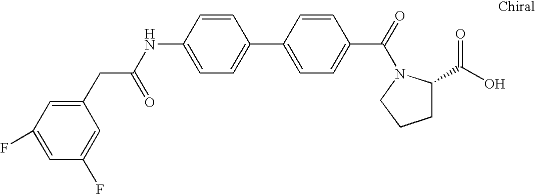 | 465.2 | 2.96 | 2 | S | 1-[(4′-{[(3,5-difluorophenyl)-acetyl]amino}- 1,1′-biphenyl-4-yl)carbonyl]-L-proline |
| 36 | 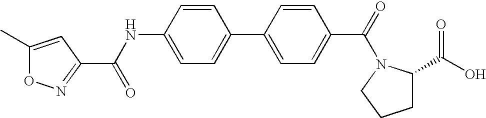 | 420.2 | 2.64 | 1 | S | 1-[(4′-{[(5-methylisoxazol-3- yl)carbonyl]amino}biphenyl-4-yl)carbonyl]- L-proline |
| 37 | 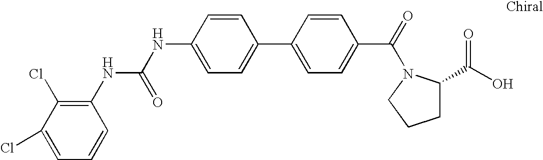 | 498.1 | 3.20 | 1 | S | 1-{[4′-({[(2,3-dichlorophenyl)- amino]carbonyl}amino)-1,1′-biphenyl-4- yl)carbonyl}-L-proline |
| 38 | 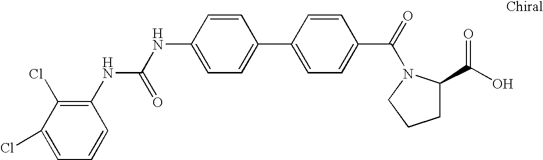 | 498.1 | 3.10 | 1 | R | 1-{[4′-({[(2,3-dichlorophenyl)- amino]carbonyl}amino)-1,1′-biphenyl-4- yl)carbonyl}-D-proline |
| 39 | 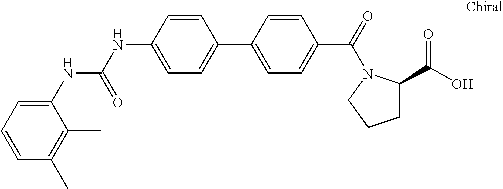 | 458.2 | 2.85 | 1 | R | 1-{[4′-({[(2,3-dimethylphenyl)- amino]carbonyl}amino)-1,1′-biphenyl-4- yl)carbonyl}-D-proline |
| 40 | 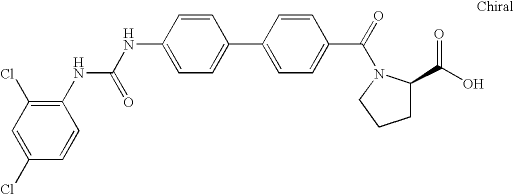 | 498.1 | 3.23 | 1 | R | 1-{[4′-({[(2,4-dichlorophenyl)- amino]carbonyl}amino)-1,1′-biphenyl-4- yl)carbonyl}-D-proline |
| 41 | 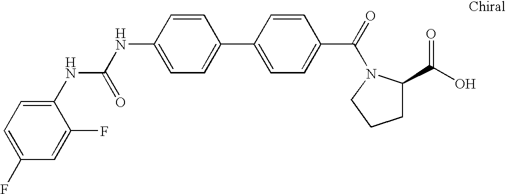 | 466.2 | 2.78 | 1 | R | 1-{[4′-({[(2,4-difluorophenyl)- amino]carbonyl}amino)-1,1′-biphenyl-4- yl)carbonyl}-D-proline |
| 42 | 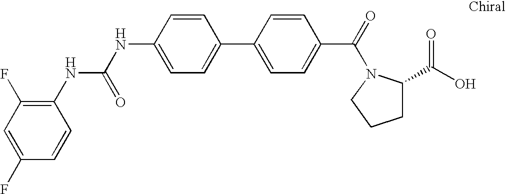 | 466.2 | 2.88 | 1 | S | 1-{[4′-({[(2,4-difluorophenyl)- amino]carbonyl}amino)-1,1′-biphenyl-4- yl)carbonyl}-L-proline |
| 43 | 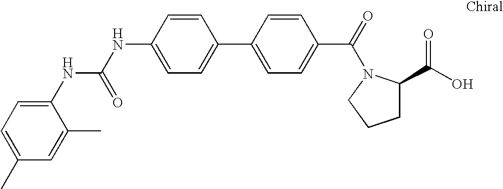 | 458.2 | 2.78 | 1 | R | 1-{[4′-({[(2,4-dimethylphenyl)- amino]carbonyl}amino)-1,1′-biphenyl-4- yl)carbonyl}-D-proline |
| 44 | 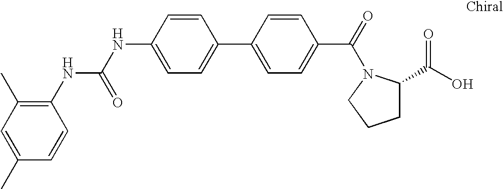 | 458.2 | 2.92 | 1 | S | 1-{[4′-({[(2,4-dimethylphenyl)- amino]carbonyl}amino)-1,1′-biphenyl-4- yl)carbonyl}-L-proline |
| 45 | 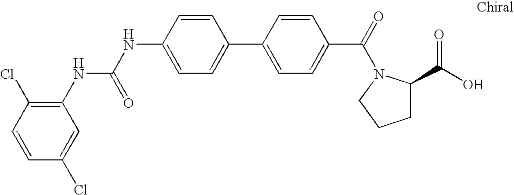 | 498.1 | 3.24 | 1 | R | 1-{[4′-({[(2,5-dichlorophenyl)- amino]carbonyl}amino)-1,1′-biphenyl-4- yl)carbonyl}-D-proline |
| 46 | 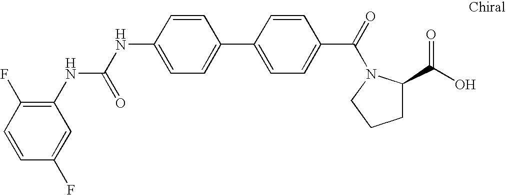 | 466.2 | 2.88 | 1 | R | 1-{[4′-({[(2,5-difluorophenyl)- amino]carbonyl}amino)-1,1′-biphenyl-4- yl)carbonyl}-D-proline |
| 47 | 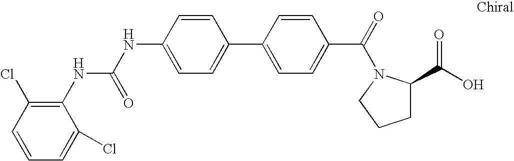 | 498.2 | 2.72 | 1 | R | 1-{[4′-({[(2,6-dichlorophenyl)- amino]carbonyl}amino)-1,1′-biphenyl-4- yl)carbonyl}-D-proline |
| 48 | 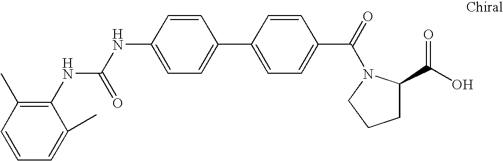 | 458.2 | 2.73 | 1 | R | 1-{[4′-({[(2,6-dimethylphenyl)- amino]carbonyl}amino)-1,1′-biphenyl-4- yl)carbonyl}-D-proline |
| 49 | 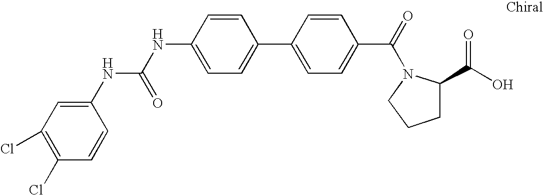 | 498.1 | 3.13 | 2 | R | 1-{[4′-({[(3,4-dichlorophenyl)- amino]carbonyl}amino)-1,1′-biphenyl-4- yl)carbonyl}-D-proline |
| 50 | 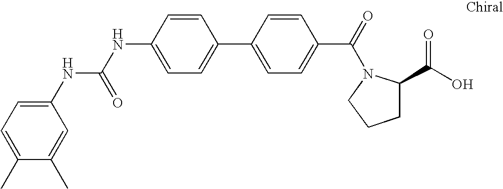 | 458.2 | 2.89 | 1 | R | 1-{[4′-({[(3,4-dimethylphenyl)- amino]carbonyl}amino)-1,1′-biphenyl-4- yl)carbonyl}-D-proline |
| 51 | 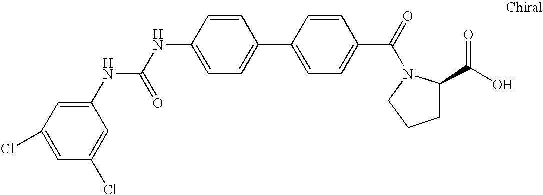 | 498.1 | 3.32 | 1 | R | 1-{[4′-({[(3,5-dichlorophenyl)- amino]carbonyl}amino)-1,1′-biphenyl-4- yl)carbonyl}-D-proline |
| 52 | 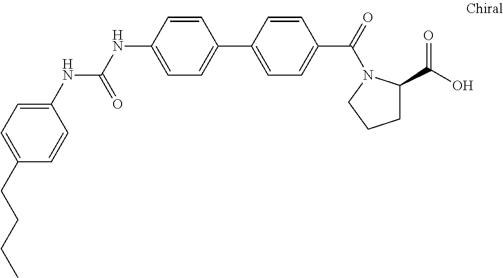 | 486.2 | 3.37 | 1 | R | 1-{[4′-({[(4-butylphenyl)- amino]carbonyl}amino)-1,1′-biphenyl-4- yl)carbonyl}-D-proline |
| 53 | 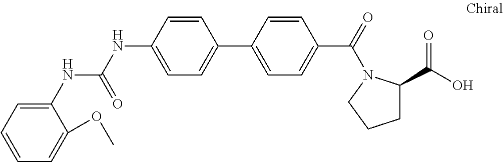 | 458.2 | 2.84 | 1 | R | 1-{[4′-({[(2-methoxyphenyl)- amino]carbonyl}amino)-1,1′-biphenyl-4- yl)carbonyl}-D-proline |
| 54 | 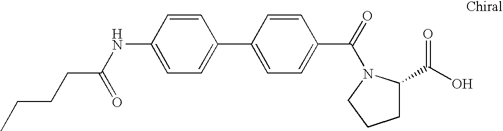 | 395.2 | 2.66 | 1 | S | 1-[{4′-(pentanoylamino]carbonyl}amino)- 1,1′-biphenyl-4- yl)carbonyl}-L-proline |
| 55 | 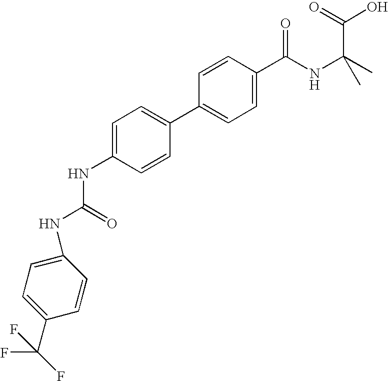 | 486.4 | 3.38 | 2 | not chiral | 2-methyl-N-({4′-[({[4- (trifluoromethyl)phenyl]amino}carbonyl) amino]biphenyl-4-yl)}carbonyl)alanine |
| 56 | 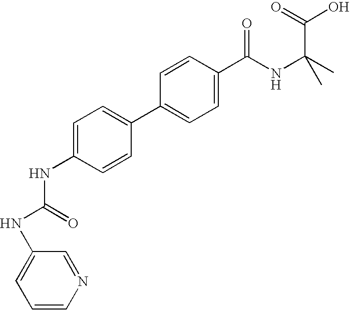 | 419.2 | 1.85 | 1 | not chiral | 2-methyl-N-[(4′-{[(pyridin-3- ylamino)carbonyl]amino}biphenyl-4- yl)carbonyl]alanine |
| 57 | 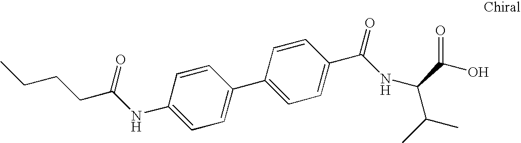 | 397.2 | 2.80 | 1 | R | N-({4′-(pentanoylamino]-1,1′-biphenyl-4- yl}carbonyl)-D-valine |
| 58 | 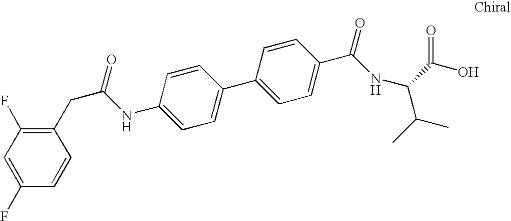 | 445.2 | 3.16 | 2 | R | N-({4′-[((2,4-difluoropheny)acetyl)amino]- 1,1′-biphenyl-4-yl}carbonyl)-L-valine |
| 59 | 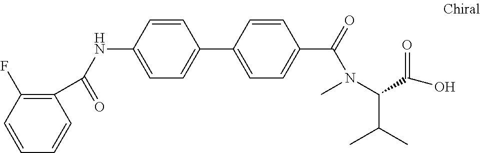 | 449.2 | 2.90 | 2 | S | N-({4′-[(2-fluorobenzoyl)amino]-1,1′- biphenyl-4-yl}carbonyl)-N-methyl-L-valine |
| 60 | 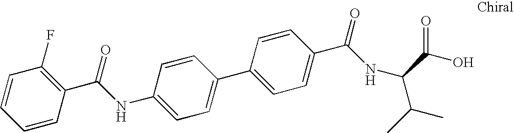 | 435.2 | 2.95 | 1 | R | N-({4′-[(2-fluorobenzoyl)amino]biphenyl-4- yl)}carbonyl)-D-valine |
| 61 | 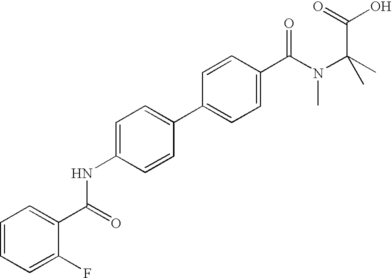 | 435.1 | 2.77 | 1 | not chiral | N-({4′-[(2-fluorobenzoyl)amino]biphenyl-4- yl}carbonyl)-N,2-dimethylalanine |
| 62 | 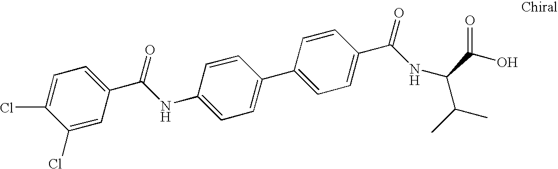 | 485.1 | 3.33 | 1 | R | N-({4′-[(3,4-dichlorobenzoyl)amino]-1,1′- biphenyl-4-yl}carbonyl)-D-valine |
| 63 | 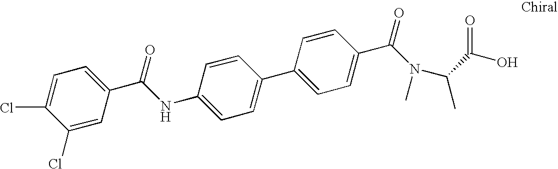 | 471.1 | 3.00 | 2 | S | N-({4′-[(3,4-dichlorobenzoyl)amino]-1,1′- biphenyl-4-yl}carbonyl)-N-methyl-L- alanine |
| 64 | 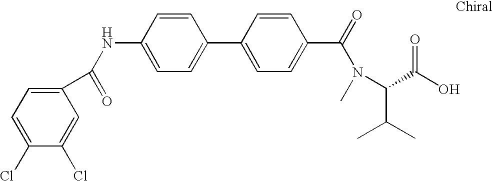 | 499.1 | 3.42 | 1 | S | N-({4′-[(3,4-dichlorobenzoyl)amino]-1,1′- biphenyl-4-yl}carbonyl)-N-methyl-L-valine |
| 65 | 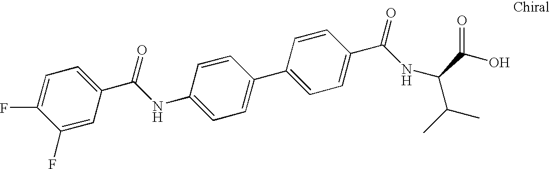 | 453.2 | 3.21 | 2 | R | N-({4′-[(3,4-difluorobenzoyl)amino]-1,1′- biphenyl-4-yl}carbonyl)-D-valine |
| 66 | 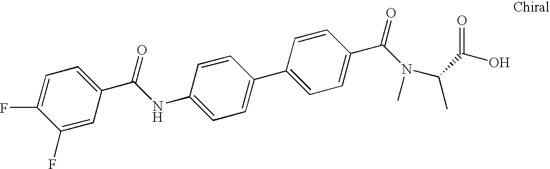 | 439.2 | 2.59 | 2 | S | N-({4′-[(3,4-difluorolbenzoyl)amino]-1,1′- biphenyl-4-yl](carbonyl)-N-methyl-L- alanine |
| 67 | 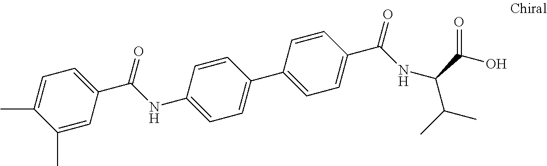 | 445.2 | 3.16 | 1 | R | N-({4′-[(3,4-dimethylbenzoyl)amino]-1,1′- biphenyl-4-yl]carbonyl)-D-valine |
| 68 | 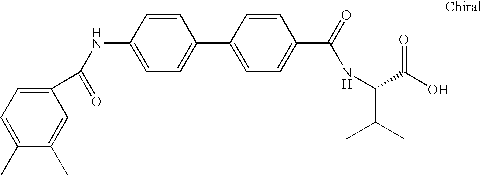 | 445.2 | 3.03 | 2 | S | N-({4′-[(3,4-dimethylbenzoyl)amino]-1,1′- biphenyl-4-yl}carbonyl)-L-valine |
| 69 | 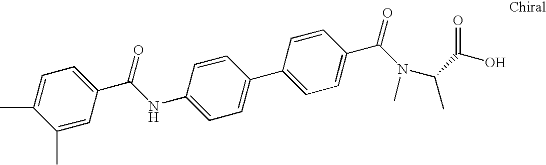 | 431.2 | 2.85 | 2 | S | N-({4′-[(3,4-dimethylbenzoyl)amino]-1,1′- biphenyl-4-yl}carbonyl)-N-methyl-L- alanine |
| 70 | 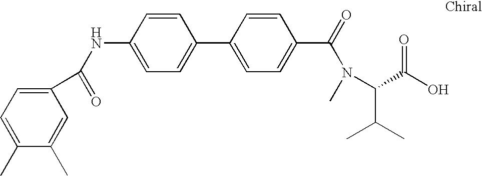 | 459.2 | 3.14 | 2 | S | N-({4′-[(3,4-dimethylbenzoyl)amino]-1,1′- biphenyl-4-yl}carbonyl)-N-methyl-L-valine |
| 71 | 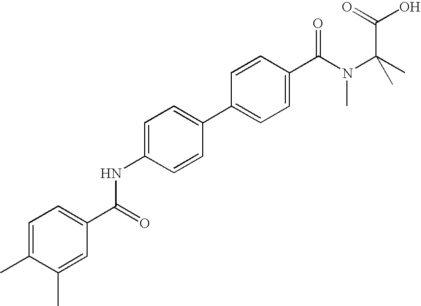 | 445.2 | 3.02 | 1 | not chiral | N-({4′-[3,4- dimethylbenzoyl)amino]biphenyl-4- yl}carbonyl)-N,2-dimethylalanine |
| 72 | 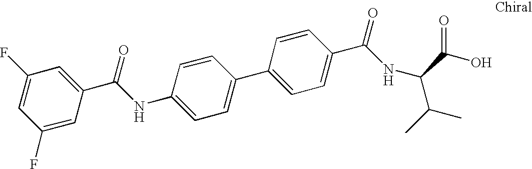 | 453.2 | 3.07 | 1 | R | N-({4′-[(3,5-difluorobenzoyl)amino]-1,1′- biphenyl-4-yl}carbonyl)-D-valine |
| 73 | 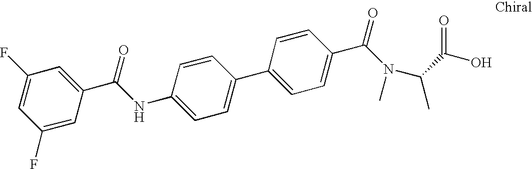 | 439.2 | 2.67 | 2 | S | N-({4′-[(3,5-difluorobenzoyl)amino]-1,1′- biphenyl-4-yl}carbonyl)-N-methyl-L- alanine |
| 74 | 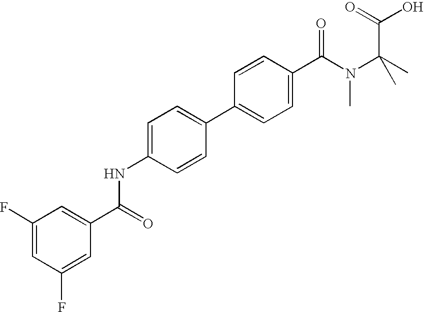 | 452.9 | 3.01 | 1 | not chiral | N-({4′-[(3,5- difluorobenzoyl)amino]biphenyl-4- yl}carbonyl)-N,2-dimethylalanine |
| 75 | 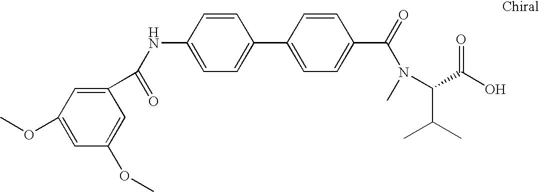 | 491.2 | 2.96 | 2 | S | N-({4′-[(3,5-dimethoxybenzoyl)amino]-1,1′- biphenyl-4-yl}carbonyl)-N-methyl-L-valine |
| 76 | 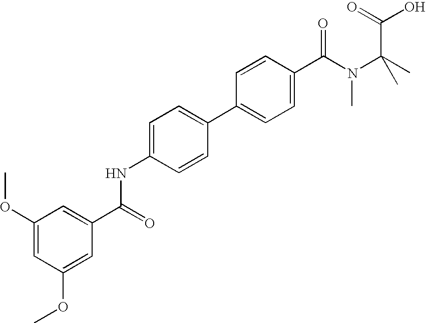 | 477.1 | 2.88 | 1 | not chiral | N-({4′-[(3,5- dimethoxybenzoyl)amino]biphenyl-4- yl}carbonyl)-N,2-dimethylalanine |
| 77 | 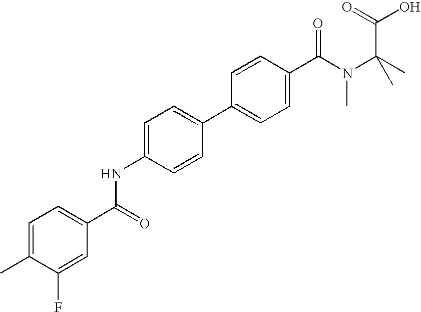 | 449.0 | 3.00 | 1 | not chiral | N-({4′-[(3-fluoro-4- methylbenzoyl)amino]biphenyl-4- yl}carbonyl)-N,2-dimethylalanine |
| 78 | 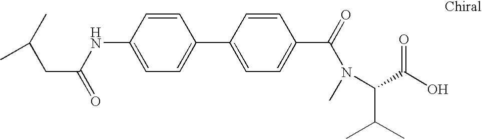 | 411.2 | 2.81 | 2 | S | N-({4′-[(3-methylbutanoyl)amino]-1,1′- biphenyl-4-yl}carbonyl)-N-methyl-L-valine |
| 79 | 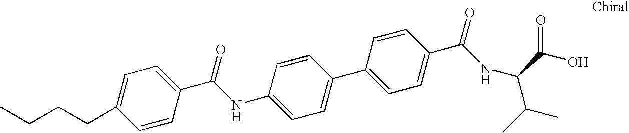 | 473.2 | 3.40 | 1 | R | N-({4′-[(4-butylbenzoyl)amino]-1,1′- biphenyl-4-yl}carbonyl)-D-valine |
| 80 | 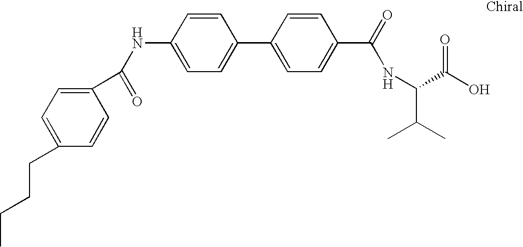 | 472.2 | 3.46 | 2 | R | N-({4′-[(4-butylbenzoyl)amino]-1,1- biphenyl-4-yl}carbonyl)-L-valine |
| 81 | 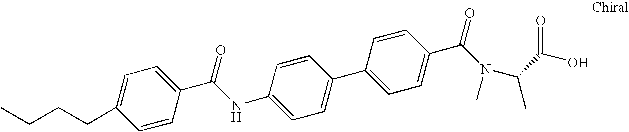 | 459.2 | 3.14 | 2 | S | N-({4′-[(4-butylbenzoyl)amino]-1,1′- biphenyl-4-yl}carbonyl)-N-methyl-L- alanine |
| 82 | 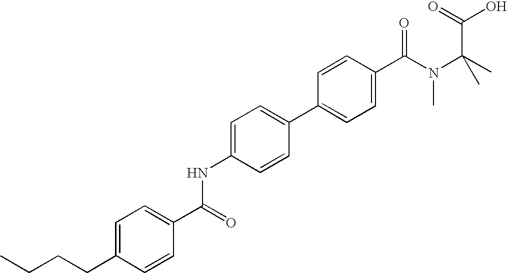 | 473.0 | 3.50 | 1 | not chiral | N-({4′-[(4-butylbenzoyl)amino]biphenyl-4- yl}carbonyl)-N,2-dimethylalanine |
| 83 | 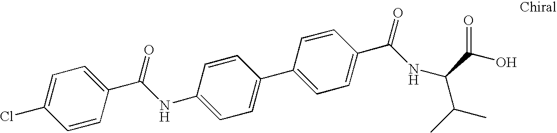 | 451.1 | 3.12 | 1 | R | N-({4′-[(4-chlorobenzoyl)amino]-1,1′- biphenyl-4-yl}carbonyl)-D-valine |
| 84 | 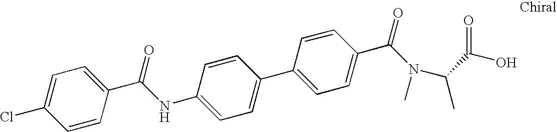 | 437.1 | 2.67 | 2 | S | N-({4′-[(4-chlorobenzoyl)amino]-1,1′- biphenyl-4-yl}carbonyl)-N-methyl-L- alanine |
| 85 | 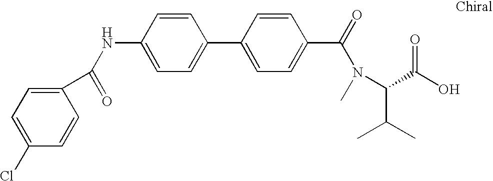 | 465.1 | 3.11 | 2 | S | N-({4′-[(4-chlorobenzoyl)amino]-1,1′- biphenyl-4-yl}carbonyl)-N-methyl-L-valine |
| 86 | 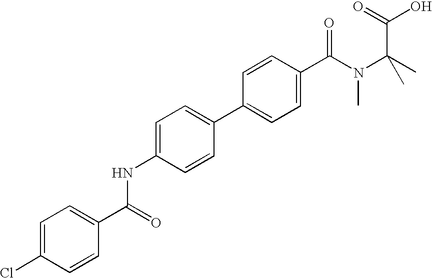 | 451.0 | 3.19 | 1 | not chiral | N-({4′-[(4-chlorobenzoyl)amino]biphenyl-4- yl}carbonyl)-N,2-dimethylalanine |
| 87 | 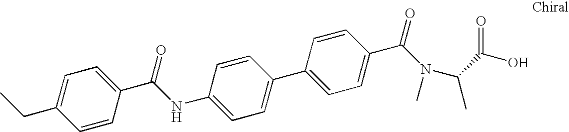 | 431.2 | 2.74 | 2 | S | N-({4′-[(4-ethylbenzoyl)amino]-1,1′- biphenyl-4-yl}carbonyl)-N-methyl-L- alanine |
| 88 | 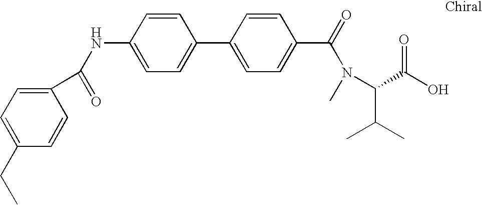 | 459.2 | 3.16 | 2 | S | N-({4′-[(4-ethylbenzoyl)amino]-1,1′- biphenyl-4-yl}carbonyl)-N-methyl-L-Valine |
| 89 | 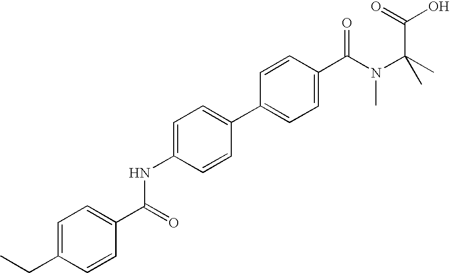 | 445.2 | 3.06 | 1 | not chiral | N-({4′-[(4-ethylbenzoyl)amino]biphenyl-4- yl}carbonyl)-N,2-dimethylalanine |
| 90 | 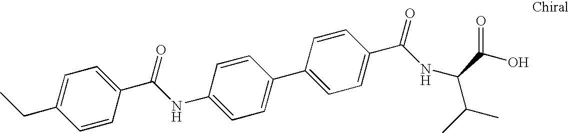 | 445.2 | 3.15 | 1 | R | N-({4′-[(4-ethyllbenzoyl)amino]-1,1′- biphenyl-4-yl}carbonyl)-D-Valine |
| 91 | 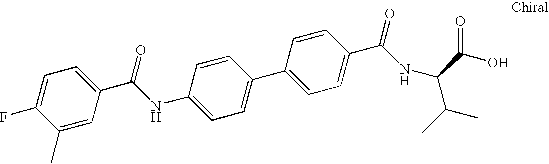 | 499.2 | 3.08 | 1 | R | N-({4′-[(4-fluoro-3-methylbenzoyl)amino]- 1,1′-biphenyl-4-yl}carbonyl)-D-Valine |
| 92 | 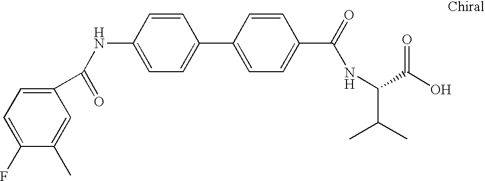 | 448.2 | 3.09 | 2 | R | N-({4′-[(4-fluoro-3-methylbenzoyl)amino]- 1,1′-biphenyl-4-yl}carbonyl)-L-valine |
| 93 | 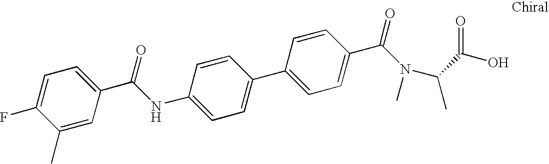 | 435.2 | 2.74 | 2 | S | N-({4′-[(4-fluoro-3-methylbenzoyl)amino]- 1,1′-biphenyl-4-yl}carbonyl)-N-methyl-L- alanine |
| 94 | 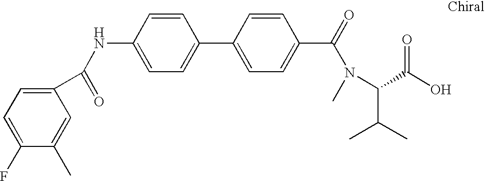 | 463.2 | 2.95 | 2 | S | N-({4′-[(4-fluoro-3-methylbenzoyl)amino]- 1,1′-biphenyl-4-yl}carbonyl)-N-methyl-L- valine |
| 95 | 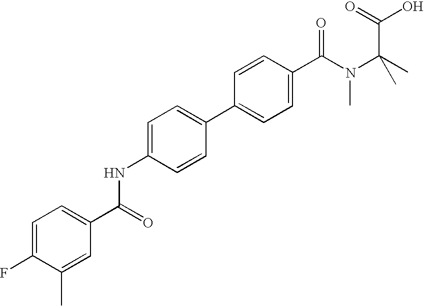 | 449.2 | 2.98 | 1 | not chiral | N-({4′-[(4-fluoro-3- methylbenzoyl)amino]biphenyl-4- yl}carbonyl)-N,2-dimethylalanine |
| 96 | 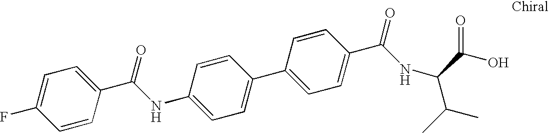 | 435.2 | 2.95 | 1 | R | N-({4′-[(4-fluorobenzoyl)amino]-1,1′- biphenyl-4-yl}carbonyl)-D-valine |
| 97 | 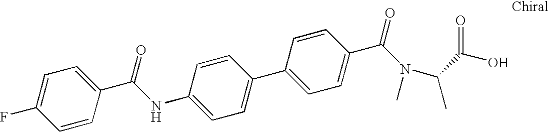 | 421.2 | 2.48 | 2 | S | N-({4′-[(4-fluorobenzoyl)amino]-1,1′- biphenyl-4-yl}carbonyl)-N-methyl-L- alanine |
| 98 | 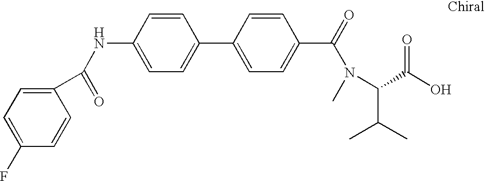 | 499.3 | 3.01 | 2 | S | N-({4′-[(4-fluorobenzoyl)amino]-1,1′- biphenyl-4-yl}carbonyl)-N-methyl-L-valine |
| 99 | 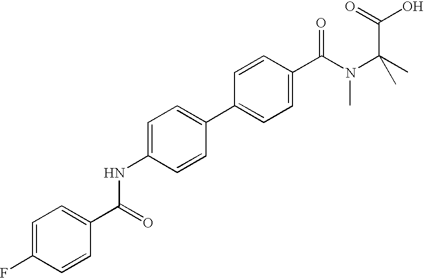 | 435.2 | 2.82 | 1 | not chiral | N-({4′-[(4-fluorobenzoyl)amino]biphenyl-4- yl}carbonyl)-N,2-dimethylalanine |
| 100 | 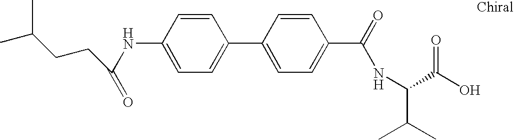 | 411.2 | 2.58 | 2 | S | N-({4′-[(4-methylpentanoyl)amino]-1,1′- biphenyl-4-yl}carbonyl)-L-valine |
| 101 | 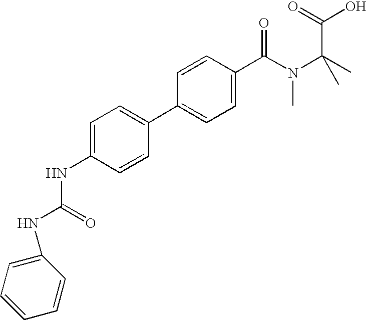 | 418.4 | 3.08 | 2 | not chiral | N-({4′-[(anilinocarbonyl)amino]biphenyl-4- yl}carbonyl)-2-methylalanine |
| 102 | 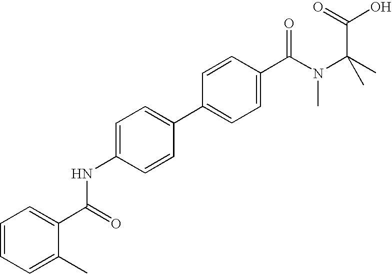 | 430.9 | 2.85 | 1 | not chiral | N,2-dimethyl-N-({4′-[(2- methylbenzoyl)amino]biphenyl-4- yl}carbonyl)alanine |
| 103 | 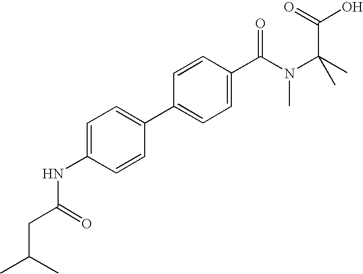 | 397.2 | 2.65 | 1 | not chiral | N,2-dimethyl-N-({4′-[(3- methylbutanoyl)amino]biphenyl-4- yl}carbonyl)alanine |
| 104 | 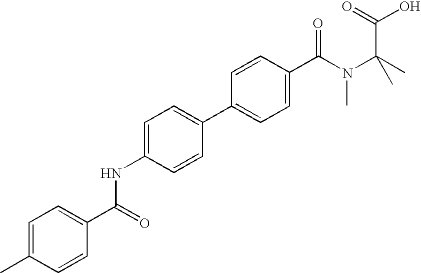 | 430.9 | 2.94 | 1 | not chiral | N,2-dimethyl-N-({4′-[(4- methylbenzoyl)amino]biphenyl-4- yl}carbonyl)alanine |
| 105 | 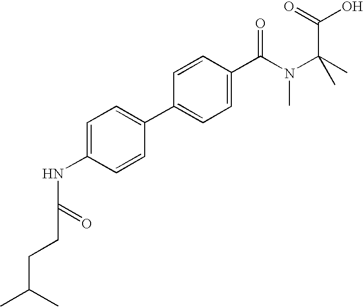 | 411.0 | 2.87 | 1 | not chiral | N,2-dimethyl-N-({4′-[(4- methylpentanoyl)amino]biphenyl-4- yl}carbonyl)alanine |
| 106 | 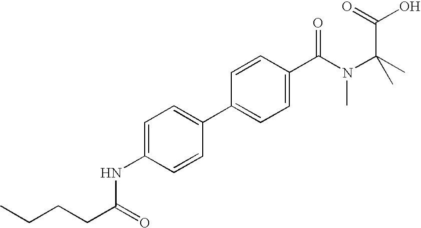 | 397.2 | 2.69 | 1 | not chiral | N,2-dimethyl-N-{[4′- (pentanoylamino)biphenyl-4- yl]carbonyl}alanine |
| 107 | 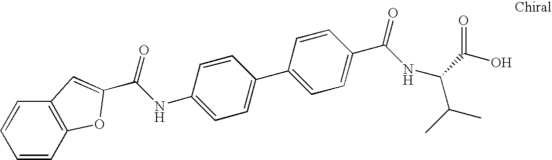 | 457.2 | 3.14 | 2 | S | N-[(4′-{[(1-benzofuran-2- yl)carbonyl]amino}-1,1′-biphenyl-4- yl)carbonyl]-L-valine |
| 108 | 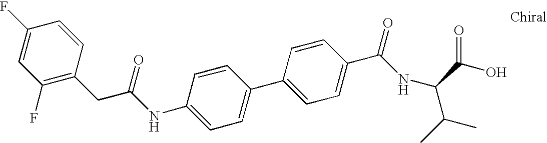 | 467.2 | 3.03 | 2 | R | N-[(4′-{[(2,4-difluorophenyl)-acetyl]amino}- 1,1′-biphenyl-4-yl)carbonyl]-D-valine |
| 109 | 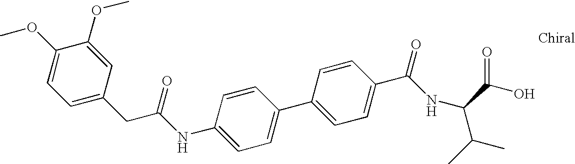 | 491.1 | 2.68 | 1 | R | N-[(4′-{[(3,4-dimethoxyphenyl)- acetyl]amino}-1,1′-biphenyl-4-yl)carbonyl] D-valine |
| 110 | 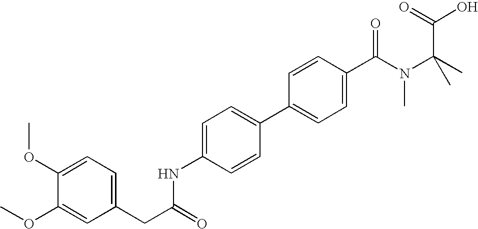 | 491.3 | 2.58 | 1 | not chiral | N-[(4′-{[(3,4-dimethoxyphenyl)- acetyl]amino}biphenyl-4-yl)carbonyl]-N,2- dimethylalanine |
| 111 | 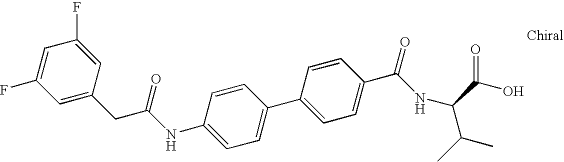 | 467.2 | 3.11 | 2 | R | N-[(4′-{[(3,5-difluorophenyl)-acetyl]amino}- 1,1′-biphenyl-4-yl)carbonyl]-D-valine |
| 112 | 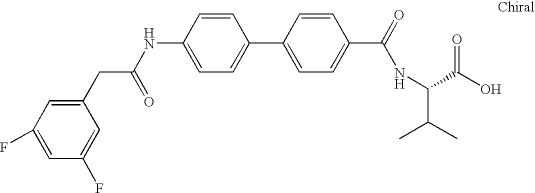 | 467.2 | 2.75 | 2 | S | N-[(4′-{[(3,5-difluorophenyl)-acetyl]amino}- 1,1′-biphenyl-4-yl)carbonyl]-L-valine |
| 113 | 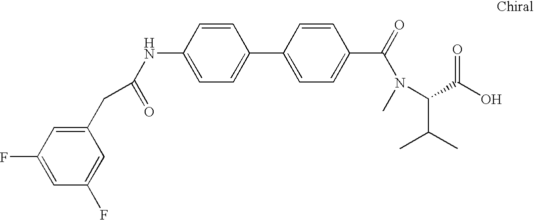 | 481.2 | 2.95 | 2 | S | N-[(4′-{[(3,5-difluorophenyl)-acetyl]amino}- 1,1′-biphenyl-4-yl)carbonyl]-N-methyl-L- valine |
| 114 | 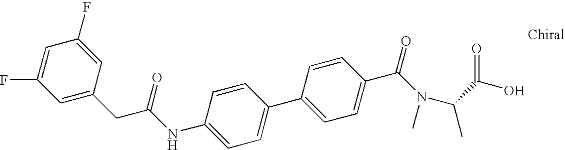 | 453.2 | 2.59 | 2 | S | N-[(4′-{[(3,5-difluorophenyl)-acetyl]amino}- 1,1′-biphenyl-4-yl)carbonyl]-N-methyl-L- alanine |
| 115 | 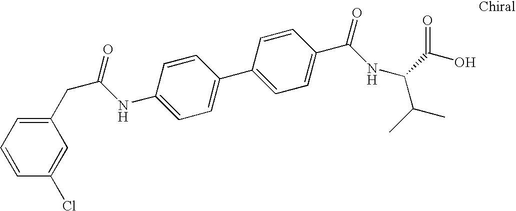 | 465.1 | 2.96 | 2.00 | S | N-[(4′-{[(3-chloroyphenyl)-acetyl]amino}- 1,1′-biphenyl-4-yl)carbonyl]-L-valine |
| 116 | 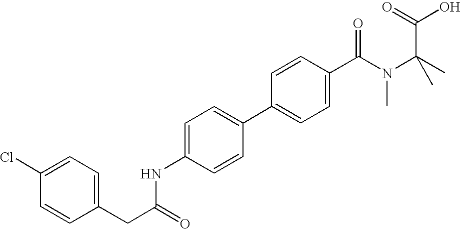 | 465.2 | 2.96 | 1 | not chiral | N-[(4′-{[(4-chlorophenyl)- acetyl]amino}biphenyl-4-yl)carbonyl]-N,2- dimethylalanine |
| 117 | 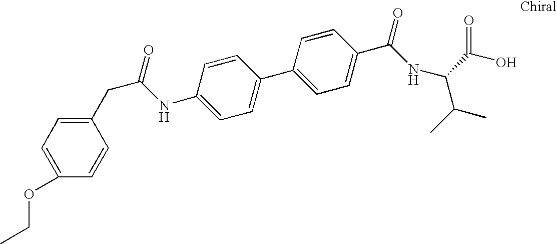 | 475.2 | 3.03 | 2 | S | N-[(4′-{[(4-ethoxyphenyl)-acetyl[amino}- 1,1′-biphenyl-4-yl)carbonyl]-L-valine |
| 118 | 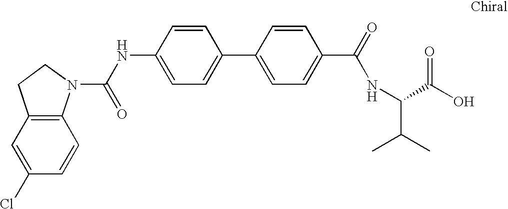 | 492.1 | 3.36 | 2 | S | N-[(4′-{[(5-chloro-2,3-dihydro-1H-indol-1- yl)carbonyl]amino}-1,1′-biphenyl-4- yl)carbonyl]-L-valine |
| 119 | 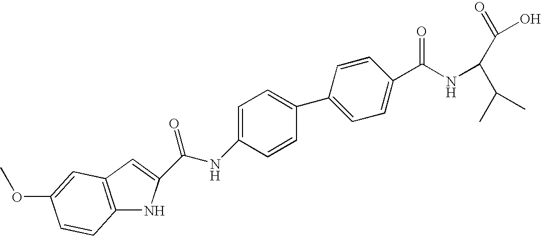 | 486.2 | 2.89 | 2 | S | N-[(4′-{[(5-methoxy-1H-indol-2- yl)carbonyl]amino}biphenyl-4-yl)carbonyl]- L-valine |
| 120 | 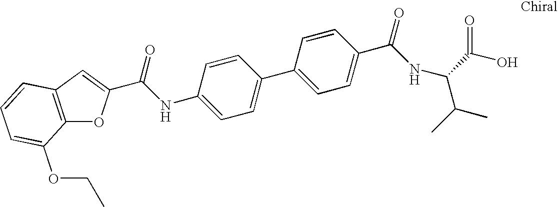 | 501.1 | 3.29 | 2 | S | N-[(4′-{[(7-ethoxy-1-benzofuran-2- yl)carbonyl]amino}-1,1′-biphenyl-4- yl)carbonyl]-L-valine |
| 121 | 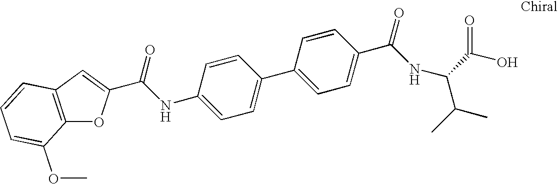 | 487.2 | 3.18 | 2 | S | N-[(4′-55 (7-methoxy-1-benzofuran-2- yl)carbonyl]amino}-1,1′-biphenyl-4- yl)carbonyl-L-valine |
| 122 | 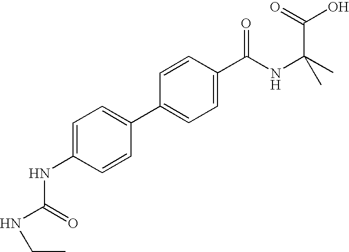 | 370.1 | 2.44 | 2 | not chiral | N-[(4′- {[(ethylamino)carbonyl]amino}biphenyl-4- yl)carbonyl]-2-methylalanine |
| 123 | 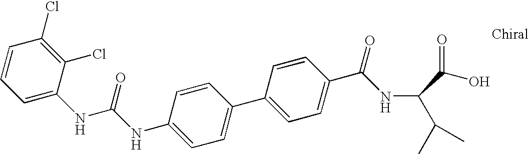 | 500.1 | 3.31 | 1 | R | N-{[4′-({[(2,3-dichlorophenyl)- amino]carbonyl}amino)-1,1′-biphenyl-4- yl]carbonyl}-D-valine |
| 124 | 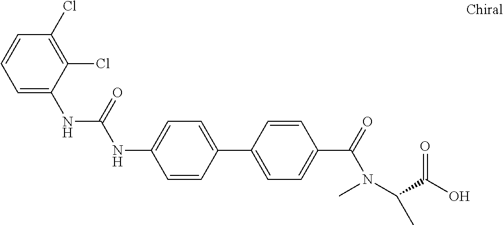 | 486.1 | 3.00 | 2 | S | N-{[4′-({[(2,3-dichlorophenyl)- amino]carbonyl}amino)-1,1′-biphenyl-4- yl]carbonyl}-N-methyl-L-alanine |
| 125 | 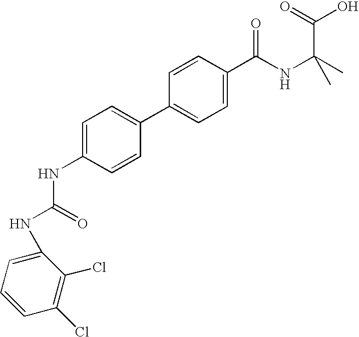 | 486.3 | 3.39 | 2 | not chiral | N-{[4′-({[(2,3-dichlorophenyl)- amino]carbonyl}amino)biphenyl-4- yl]carbonyl}-2-methy[alanine |
| 126 | 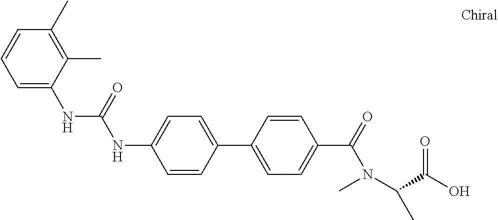 | 446.2 | 2.37 | 2 | S | N-{[4′-({[(2,3-dimethylphenyl)- amino]carbonyl}amino)-1,1′-biphenyl-4- yl]carbonyl}-N-methyl-L-alanine |
| 127 | 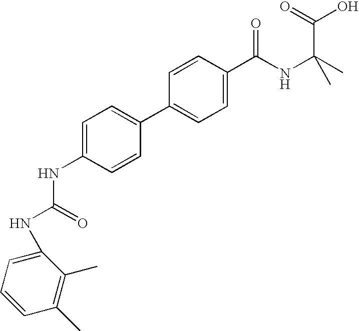 | 446.1 | 2.92 | 2 | not chiral | N-{[4′-({[(2,3-dimethylphenyl)- amino]carbonyl}amino)-biphenyl-4- yl]carbonyl}-2-methylalanine |
| 128 | 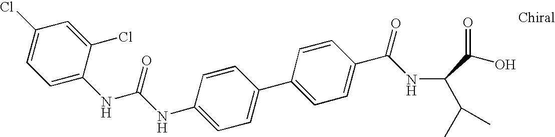 | 500.1 | 3.37 | 1 | R | N-{[4′-({[(2,4-dichlorophenyl)- amino]carbonyl}amino)-1,1′-biphenyl-4- yl]carbonyl}-D-valine |
| 129 | 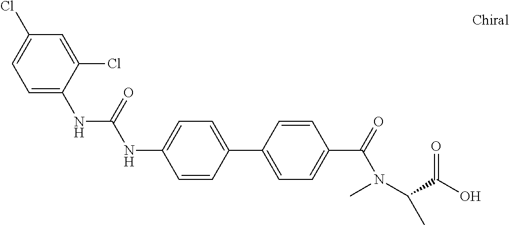 | 486.1 | 2.96 | 2 | S | N-{4′-({[(2,4-dichlorophenyl)- amino]carbonyl}amino)-1,1′-biphenyl-4- yl]carbonyl}-N-methyl-L-alanine |
| 130 | 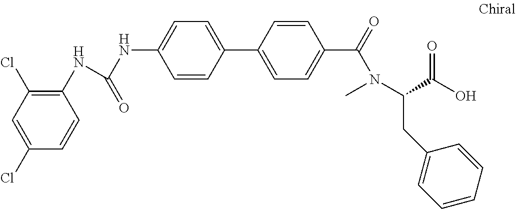 | 563.1 | 3.29 | 2 | S | N-{[4′-({[(2,4-dichlorophenyl)- amino]carbonyl}amino)-1,1′-biphenyl-4- yl]carbonyl}-N-methyl-L-phenylalanine |
| 131 | 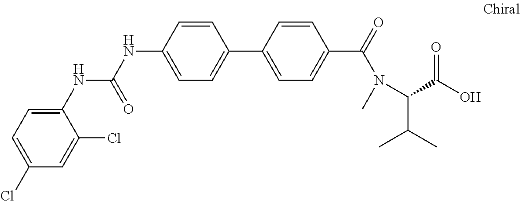 | 514.1 | 3.47 | 1 | S | N-{[4′-({[(2,4-dichlorophenyl)- amino]carbonyl}amino)-1,1′-biphenyl-4- yl]carbonyl}-N-methyl-L-valine |
| 132 | 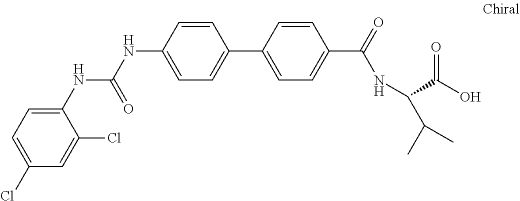 | 501.1 | 3.44 | 2 | S | N-{[4′-({[(2,4-dichlorophenyl)- amino]carbonyl}amino)-1,1′-biphenyl-4- yl]carbonyl}-L-valine |
| 133 | 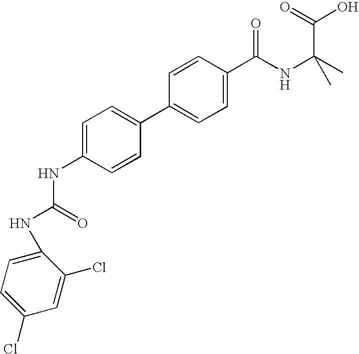 | 486.0 | 3.31 | 2 | not chiral | N-{[4′-({[(2,4-dichlorophenyl)- amino)carbonyl}amino)biphenyl-4- yl]carbonyl}-2-methylalanine |
| 134 | 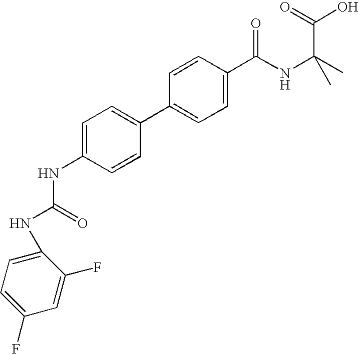 | 454.0 | 2.92 | 2 | not chiral | N-{[4′-({[(2,4-diflourophenyl)- amino]carbonyl}amino)biphenyl-4- yl]carbonyl}-2-methylalanine |
| 135 | 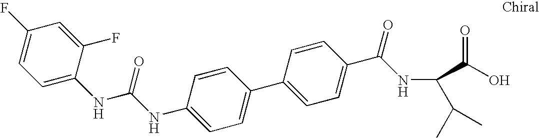 | 468.2 | 3.04 | 1 | R | N-{[4′-({[(2,4-difluorophenyl)- amino]carbonyl}amino)-1,1′-biphenyl-4- yl]carbonyl}-D-valine |
| 136 | 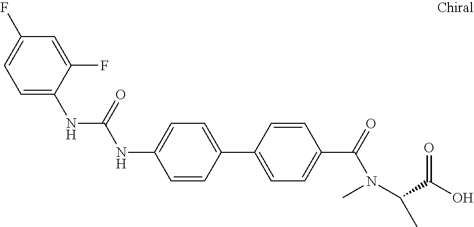 | 454.2 | 2.74 | 2 | S | N-{[4′-({[(2,4-difluorophenyl)- amino]carbonyl}amino)-1,1′-biphenyl-4- yl]carbonyl}-N-methyl-L-alanine |
| 137 | 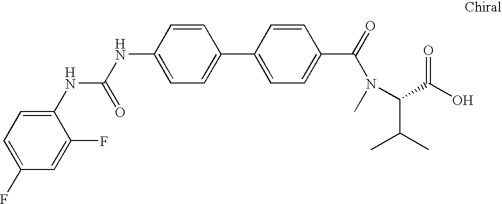 | 481.2 | 3.01 | 1 | S | N-{[4′-(2,4-difluorophenyl)- amino]carbonyl}amino)-1,1′-biphenyl-4- yl]carbonyl}-N-methyl-L-valine |
| 138 | 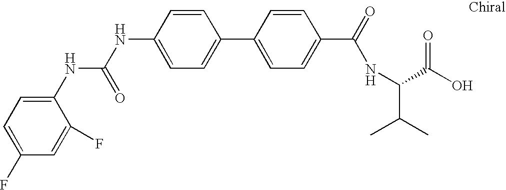 | 468.2 | 3.06 | 2 | S | N-{[4′-({[(2,4-difluorophenyl)- amino]carbonyl}amino)-1,1′-biphenyl-4- yl]carbonyl}-L-valine |
| 139 | 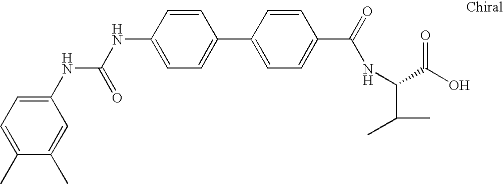 | 460.2 | 3.24 | 2 | S | N-{[4′-({[(3,4-dimethylphenyl)- amino]carbonyl}amino)-1,1′-biphenyl-4- yl]carbonyl}-L-valine |
| 140 | 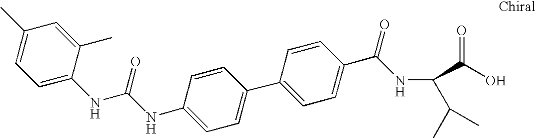 | 460.2 | 3.08 | 2 | R | N-{[4′-({[(2,4-dimethylphenyl)- amino]carbonyl}amino)-1,1′-biphenyl-4- yl]carbonyl}-D-valine |
| 141 | 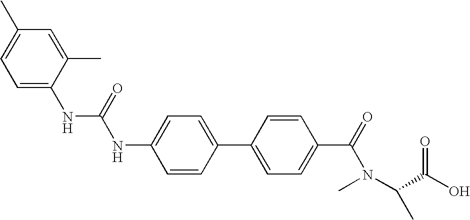 | 446.2 | 1.58 | 2 | S | N-{[4′-({[(2,4-dimethylphenyl)- amino]carbonyl}amino)-1,1′-biphenyl-4- yl]carbonyl}-N-methyl-L-alanine |
| 142 | 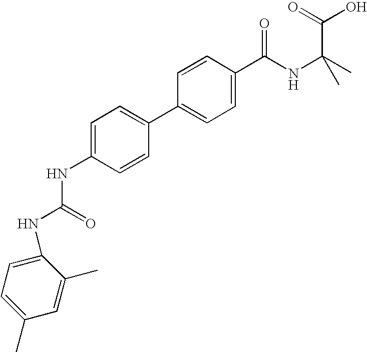 | 446.1 | 3.03 | 2 | not chiral | N-{[4′-({[(2,4-dimethylphenyl)- amino]carbonyl}amino)biphenyl-4- yl]carbonyl}-2-methylalanine |
| 143 | 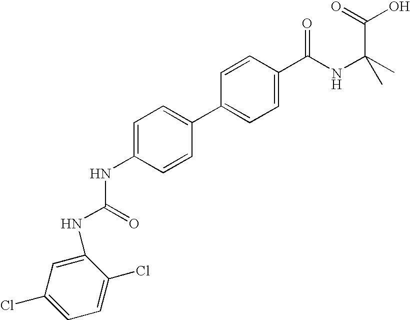 | 486.3 | 3.45 | 2 | not chiral | N-{[4′-({[(2,5-dichlorophenyl)- amino]carbonyl}amino)biphenyl-4- yl]carbonyl}-2-methylalanine |
| 144 | 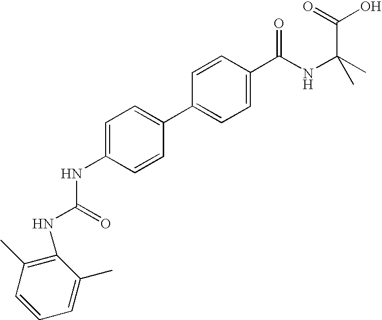 | 446.2 | 2.74 | 2 | not chiral | N-{[4′-({[(2,6-dimethyhlphenyl)- amino]carbonyl}amino)biphenyl-4- yl]carbonyl}-2-methylalanine |
| 145 | 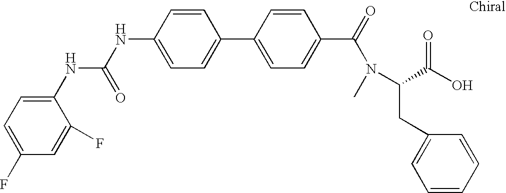 | 530.2 | 2.92 | 2 | S | N-{[4′-({[(2,4-difluorophenyl)- amino]carbonyl}amino)-1,1′-biphenyl-4- yl]carbonyl}-N-methyl-L-phenylalanine |
| 146 | 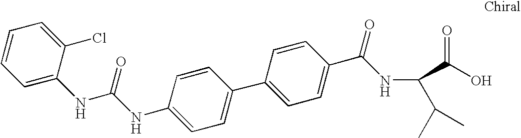 | 466.1 | 3.10 | 1 | R | N-{[4′-({[(2-chlorophenyl)- amino]carbonyl}amino)-1,1′-biphenyl-4- yl]carbonyl}-D-valine |
| 147 | 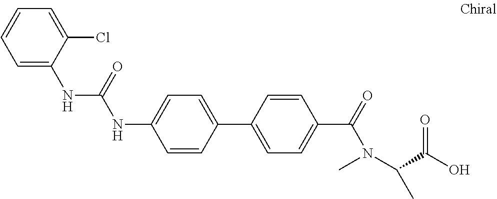 | 452.1 | 2.26 | 2 | S | N-{[4′-({[(2-chlorophenyl)- amino]carbonyl}amino)-1,1′-biphenyl-4- yl]carbonyl}-N-methyl-L-alanine |
| 148 | 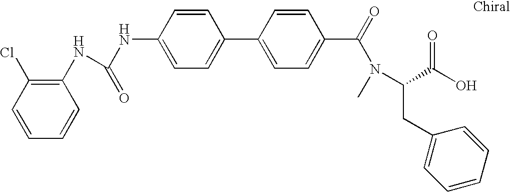 | 529.1 | 3.01 | 2 | S | N-{[4′-({[(2-chlorophenyl)- amino]carbonyl}amino)-1,1′-biphenyl-4- yl]carbonyl}-N-methyl-L-phenylalanine |
| 149 | 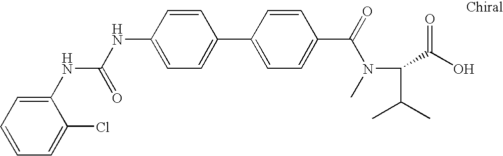 | 480.1 | 3.13 | 1 | S | N-{[4-({[(2-chlorophenyl)- amino]carbonyl}amino)-1,1′-biphenyl-4- yl]carbonyl}-N-methyl-L-valine |
| 150 | 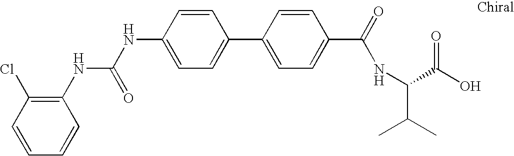 | 466.1 | 3.10 | 2 | S | N-{[4-({[(2-chlorophenyl)- amino]carbonyl}amino)-1,1′-biphenyl-4- yl]carbonyl}-L-valine |
| 151 | 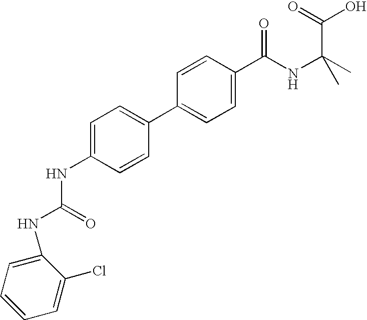 | 452.0 | 3.03 | 2 | not chiral | N-{[4′-({[(2-chlorophenyl)- amino]carbonyl}amino)biphenyl-4- yl]carbonyl}-2-methylalanine |
| 152 | 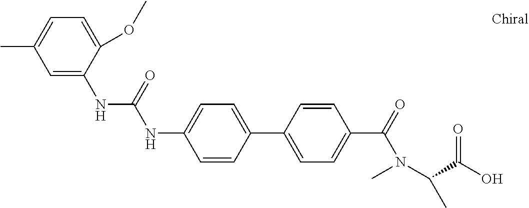 | 462.2 | 2.52 | 2 | S | N-{[4′-({[(2-methoxy-5-methylphenyl)- amino]carbonyl}amino)-1,1′-biphenyl-4- yl]carbonyl}-N-methyl-L-alanine |
| 153 | 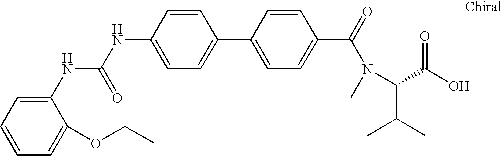 | 490.2 | 3.18 | 1 | S | N-{[4′-({[(2-ethoxyphenyl)- amino]carbonylamino)-1,1′-biphenyl-4- yl]carbonyl}-N-methyl-L-valine |
| 154 | 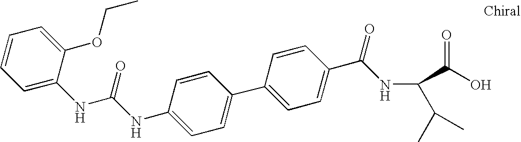 | 476.2 | 3.22 | 2 | R | N-{[4′-({[(2-ethoxyphenyl)- amino]carbonyl}amino)-1,1′-biphenyl-4- yl]carbonyl}-D-valine |
| 155 | 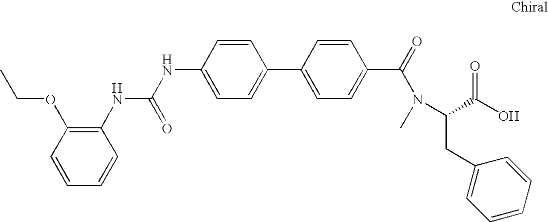 | 538.2 | 2.97 | 2 | S | N-{[4′-({[(2-ethoxyphenyl)- amino]carbonyl}amino)-1,1′-biphenyl-4- yl]carbonyl}-N-methyl-L-phenylalanine |
| 156 | 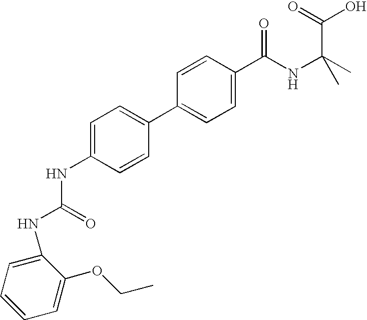 | 462.1 | 3.00 | 2 | not chiral | N-{[4′-({[(2-ethoxyphenyl)- amino]carbonyl}amino)biphenyl-4- yl]carbonyl}-2-methylalanine |
| 157 | 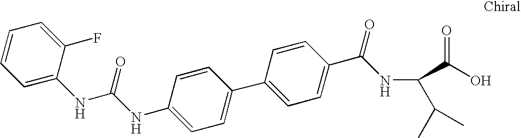 | 450.2 | 3.00 | 2 | R | N-{[4′-({[(2-fluorophenyl)- amino]carbonyl}amino)-1,1′-biphenyl-4- yl]carbonyl}-D-valine |
| 158 | 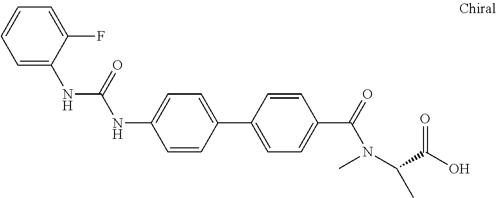 | 436.2 | 2.26 | 2 | S | N-{[4′-({[(2-fluorophenyl)- amino]carbonyl}amino)-1,1′-biphenyl-4- yl]carbonyl}-N-methyl-L-alanine |
| 159 | 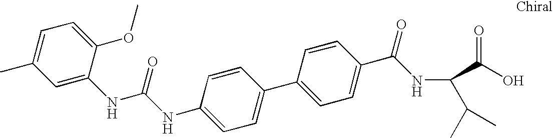 | 476.2 | 3.22 | 2 | R | N-{[4′-({[(2-methoxy-5-methylphenyl)- amino]carbonyl}amino)-1,1′-biphenyl-4- yl]carbonyl}-D-valine |
| 160 | 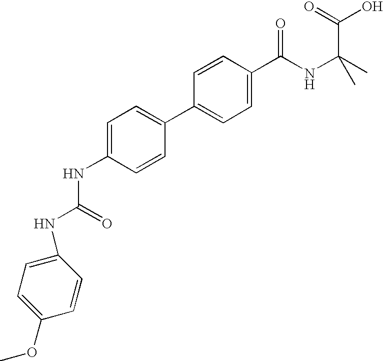 | 448.4 | 3.02 | 2 | not chiral | N-{[4′-({[(4-methoxyphenyl)- amino]carbonyl}amino)biphenyl-4- yl]carbonyl}-2-methylalanine |
| 161 | 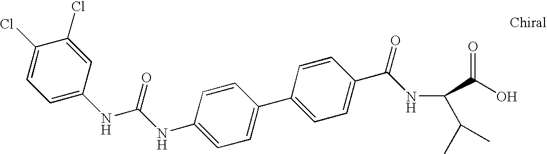 | 500.1 | 3.39 | 1 | R | N-{[4′-({[(3,4-dichlorophenyl)- amino]carbonyl}amino)-1,1′-biphenyl-4- yl]carbonyl}-D-valine |
| 162 | 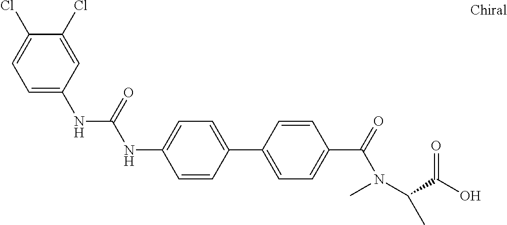 | 486.1 | 2.92 | 2 | S | N-{[4′-({[(3,4-dichlorophenyl)- amino]carbonyl}amino)-1,1′-biphenyl-4- yl]carbonyl}-N-methyl-L-alanine |
| 163 | 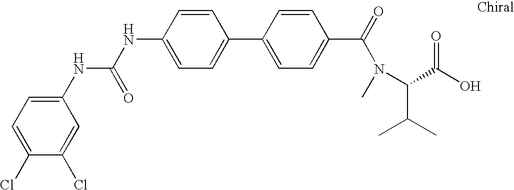 | 514.1 | 3.45 | 1 | S | N-{[4′-({[(3,4-dichlorophenyl)- amino]carbonyl}amino)-1,1′-biphenyl-4- yl]carbonyl}-N-methyl-L-valine |
| 164 | 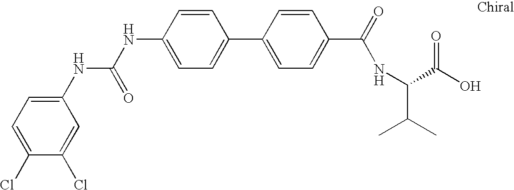 | 501.1 | 3.47 | 2 | S | N-{[4′-({[(3,4-dichlorophenyl)- amino]carbonyl}amino)-1,1′-biphenyl-4- yl]carbonyl}-L-valine |
| 165 | 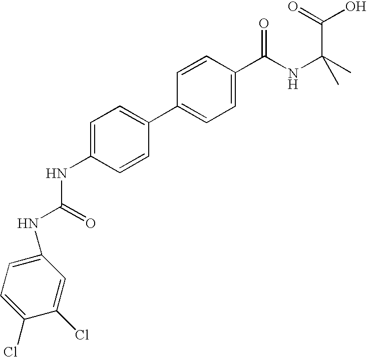 | 486.3 | 3.39 | 2 | not chiral | N-{[4′-({[(3,4-dichlorophenyl)- amino]carbonyl}amino)biphenyl-4- yl]carbonyl}-2-methylalanine |
| 166 | 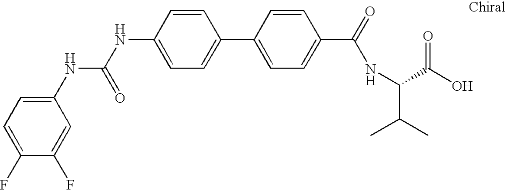 | 468.2 | 3.02 | 2 | S | N-{[4′-({[(3,4-difluorophenyl)- amino]carbonyl}amino)-1,1′-biphenyl-4- yl]carbonyl}-L-valine |
| 167 | 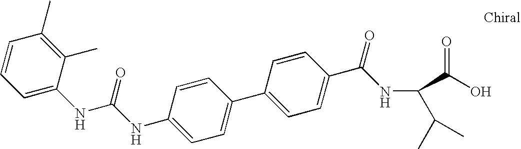 | 460.2 | 3.07 | 2 | R | N-{[4′-({[(2,3-dimethylphenyl)- amino]carbonyl}amino)-1,1′-biphenyl-4- yl]carbonyl}-D-valine |
| 168 | 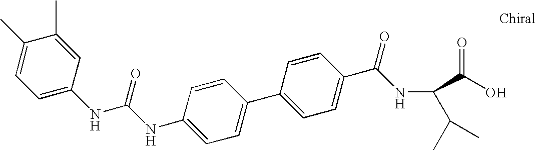 | 460.2 | 3.10 | 1 | R | N-{[4′-({[(3,4-dimethylphenyl)- amino]carbonyl}amino)-1,1′-biphenyl-4- yl]carbonyl}-D-valine |
| 169 | 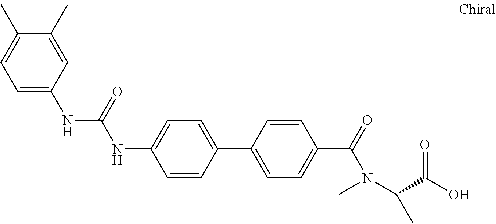 | 446.2 | 2.48 | 2 | S | N-{[4′-({[(3,4-dimethylphenyl)- amino]carbonyl}amino)-1,1′-biphenyl-4- yl]carbonyl}-N-methyl-L-alanine |
| 170 | 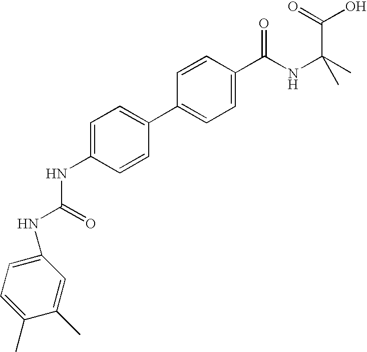 | 446.1 | 3.03 | 2 | not amino | N-{[4′-({[(3,4-dimethylphenyl)- amino]carbonyl}amino)biphenyl-4- yl]carbonyl)-2-methylalanine |
| 171 | 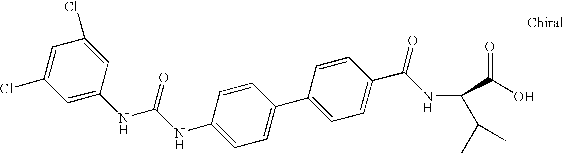 | 500.1 | 3.44 | 2 | R | N-{[4′-({[(3,5-dichlorophenyl)- amino]carbonyl}amino)-1,1′-biphenyl-4- yl]carbonyl}-D-valine |
| 172 | 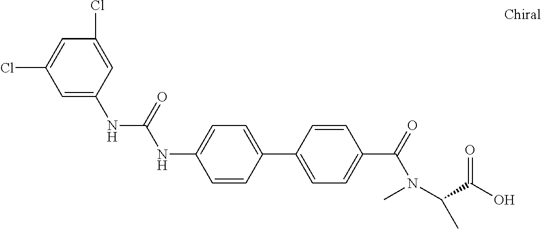 | 486.1 | 3.17 | 1 | S | N-{[4′-({[(3,5-dichlorophenyl)- amino]carbonyl}amino)-1,1′-biphenyl-4- yl]carbonyl}-N-methyl-L-alanine |
| 173 | 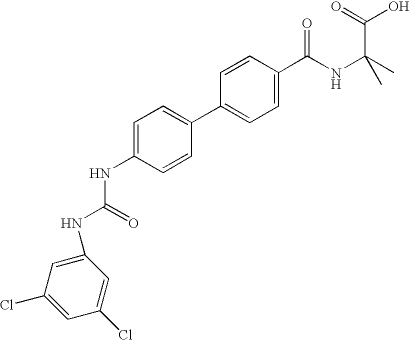 | 486.3 | 3.48 | 2 | not chiral | N-{[4-({[(3,5-dichlorophenyl)- amino]carbonyl}amino)biphenyl-4- yl]carbonyl}-2-methylalanine |
| 174 | 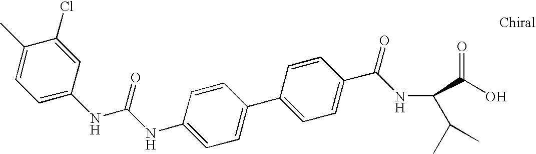 | 480.2 | 3.29 | 2 | R | N-{[4′-({[(3-chloro-4-methylphenyl)- amino]carbonyl}amino)-1,1′-biphenyl-4- yl]carbonyl}-D-valine |
| 175 | 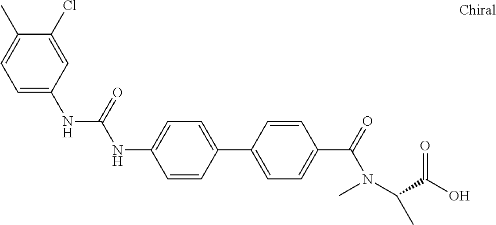 | 466.2 | 2.99 | 1 | S | N-{[4′-({[(3-chloro-4-methylphenyl)- amino]carbonyl}amino)-1,1-biphenyl-4- yl]carbonyl}-N-methyl-L-alanine |
| 176 | 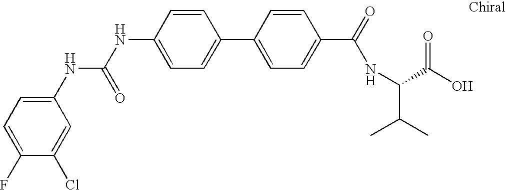 | 460.2 | 3.37 | 2 | S | N-{[4′-({[(3-chloro-4-methylphenyl)- amino]carbonyl}amino)-1,1′-biphenyl-4- yl]carbonyl}-L-valine |
| 177 | 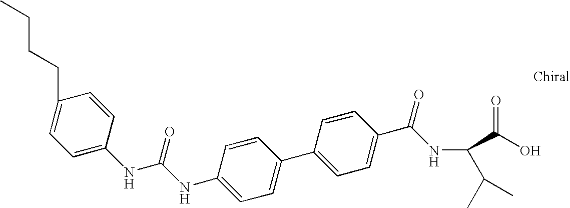 | 488.2 | 3.46 | 1 | R | N-{[4′-({[(4-butylphenyl)- amino]carbonyl}amino)-1,1′biphenyl-4- yl]carbonyl}-D-valine |
| 178 | 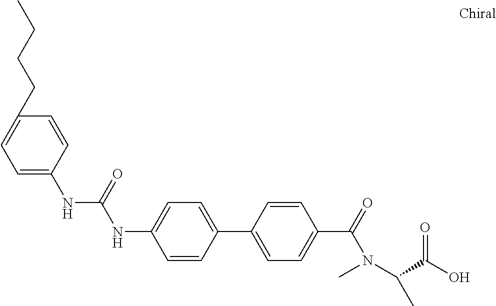 | 474.2 | 2.95 | 2 | S | N-{[4′-({[(4-butylphenyl)- amino]carbonyl}amino)-1,1-biphenyl-4- yl]carbonyl}-N-methyl-L-alanine |
| 179 | 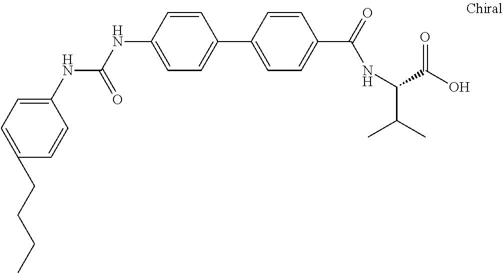 | 488.2 | 3.45 | 2 | S | N-{[4′-({[(4-butylphenyl)- amino]carbonyl}amino)-1,1′-biphenyl-4- yl]carbonyl}-L-valine |
| 180 | 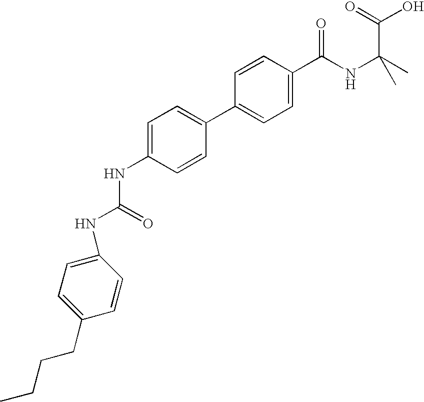 | 474.1 | 3.40 | 2 | not chiral | N-{[4′-({[(4-butylphenyl)- amino]carbonyl}amino)biphenyl-4- yl]carbonyl}-2-methylalanine |
| 181 | 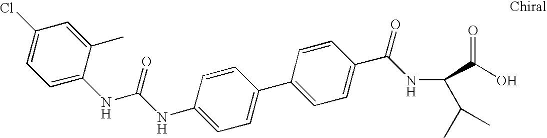 | 480.2 | 3.18 | 1 | R | N-{[4′-({[(4-chloro-2-methylphenyl)- amino]carbonyl}amino)-1,1′-biphenyl-4- yl]carbonyl}-D-valine |
| 182 | 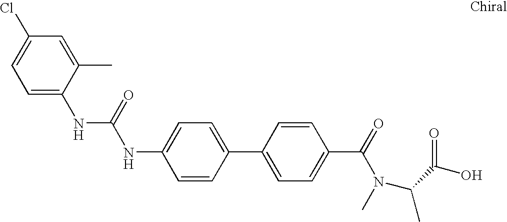 | 466.2 | 2.56 | 2 | S | N-{[4′-({[(4-chloro-2-methylphenyl)- amino]carbonyl}amino)-1,1′-biphenyl-4- yl]carbonyl}-N-methyl-L-alanine |
| 183 | 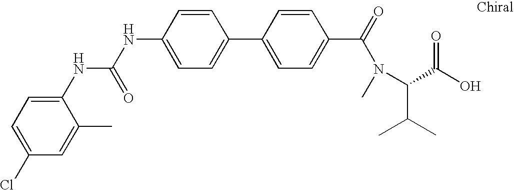 | 494.2 | 3.21 | 1 | S | N-{[4′-({[(4-chloro-2-methylphenyl)- amino]carbonyl}amino)-1,1′-biphenyl-4- yl]carbonyl}-N-methyl-L-valine |
| 184 | 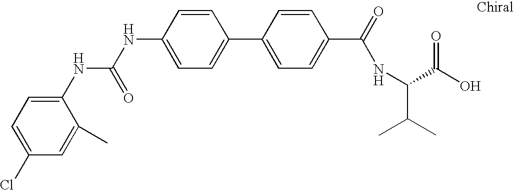 | 480.2 | 3.24 | 2 | S | N-{[4′-({[(4-chloro-2-methylphenyl)- amino]carbonyl}amino)-1,1′-biphenyl-4- yl]carbonyl}-L-valine |
| 185 | 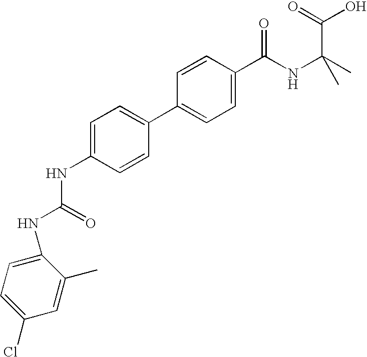 | 466.1 | 3.07 | 2 | not chiral | N-{[4′-({[(4-chloro-2-methylphenyl)- amino]carbonyl}amino)biphenyl-4- yl]carbonyl}-2-methylalanine |
| 186 | 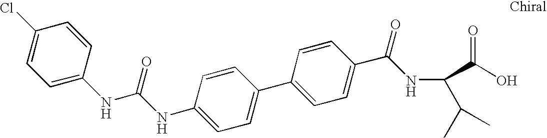 | 466.1 | 3.12 | 1 | R | N-{[4′-({[(4-chlorophenyl)- amino]carbonyl}amino)-1,1′-biphenyl-4- yl]carbonyl}-D-valine |
| 187 | 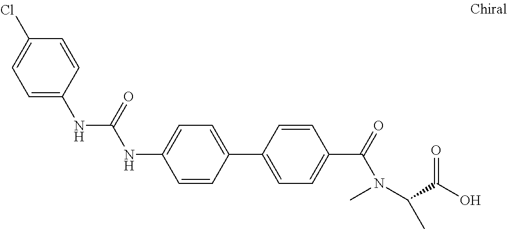 | 452.1 | 2.70 | 2 | S | N-{[4′-({[(4-chlorophenyl)- amino]carbonyl}amino)-1,1′-biphenyl-4- yl]carbonyl}-N-methyl-L-alanine |
| 188 | 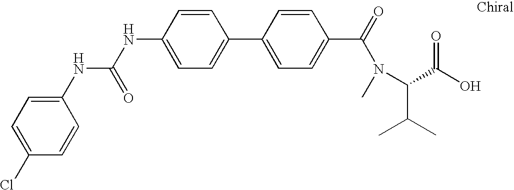 | 480.1 | 3.20 | 1 | S | N-{[4′-({[(4-chlorophenyl)- amino]carbonyl}amino)-1,1′-biphenyl-4- yl]carbonyl)-N-methyl-L-valine |
| 189 | 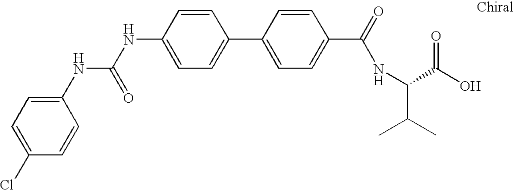 | 466.1 | 3.23 | 2 | S | N-{[4′-({[(4-chlorophenyl)- amino]carbonyl}amino)-1,1′-biphenyl-4- yl]carbonyl}-L-valine |
| 190 | 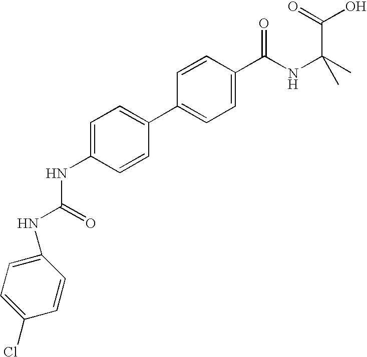 | 452.0 | 3.05 | 2 | not chiral | N-{[4′-({[(4-chlorophenyl)- amino]carbonyl}amino)biphenyl-4- yl]carbonyl}-2-methylalanine |
| 191 | 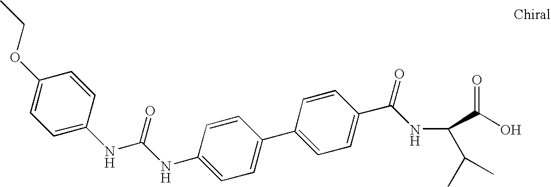 | 477.2 | 3.03 | 2 | R | N-{[4′-({[(4-ethoxyphenyl)- amino]carbonyl}amino)-1,1′-biphenyl-4- yl]carbonyl}-D-valine |
| 192 | 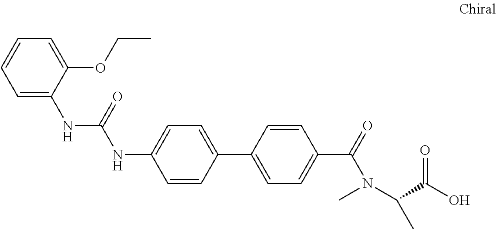 | 462.2 | 1.92 | 2 | S | N-{[4′-({[(2-ethoxyphenyl)- amino]carbonyl}amino)-1,1′-biphenyl-4- yl]carbonyl}-N-methyl-L-alanine |
| 193 | 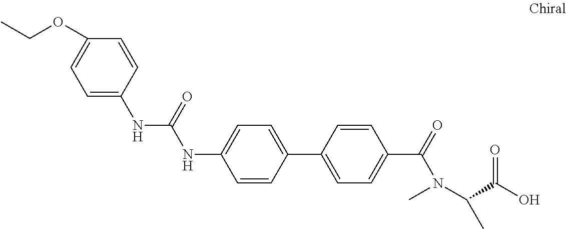 | 462.2 | 2.69 | 1 | S | N-{[4′-({[(4-ethoxyphenyl)- amino]carbonyl}amino)-1,1′-biphenyl-4- yl]carbonyl}-N-methyl-L-alanine |
| 194 | 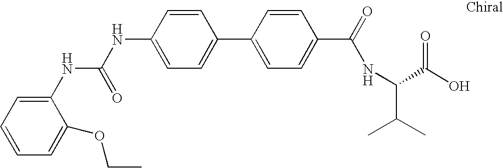 | 476.2 | 3.14 | 2 | S | N-{[4′-({[(2-ethoxyphenyl)- amino]carbonyl}amino)-1,1′-biphenyl-4- yl]carbonyl}-L-valine |
| 195 | 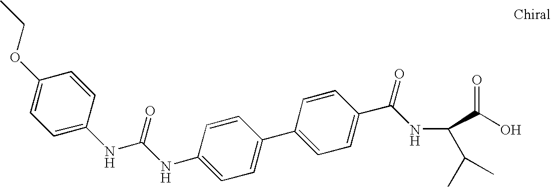 | 460.2 | 3.14 | 2 | R | N-{[4′-({[(4-ethylphenyl)- amino]carbonyl}amino)-1,1′-biphenyl-4- yl]carbonyl}-D-valine |
| 196 | 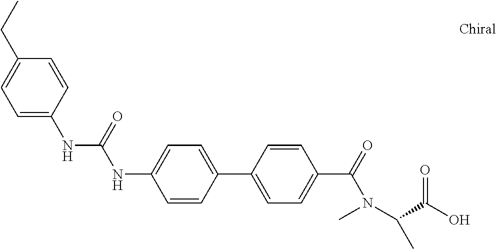 | 446.2 | 2.56 | 2 | S | N-{[4-({[(4-ethylphenyl)- amino)carbonyl}amino)-1,1′-biphenyl-4- yl]carbonyl}-N-methyl-L-alanine |
| 197 | 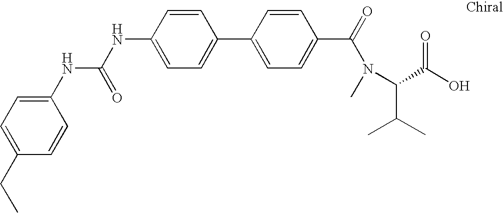 | 474.2 | 3.24 | 1 | S | N-{[4′-({[(4-ethylphenyl)- amino]carbonyl}amino)-1,1′-biphenyl-4- yl]carbonyl}-N-methyl-L-valine |
| 198 | 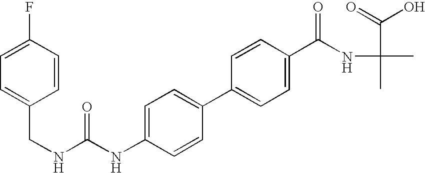 | 450.4 | 3.05 | 2 | not chiral | N-{[4′-({[(4- fluorobenzyl)amino]carbonyl}amino)biphenyl- 4-yl]carbonyl}-2-methylalanine |
| 199 | 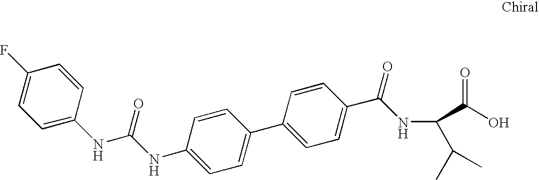 | 450.2 | 3.00 | 2 | R | N-{[4′-({[(4-fluorophenyl)- amino]carbonyl}amino)-1,1′-biphenyl-4- yl]carbonyl}-D-valine |
| 200 | 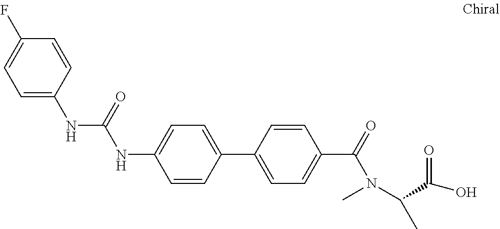 | 436.2 | 2.66 | 1 | S | N-{[4′-({[(4-fluorophenyl)- amino]carbonyl}amino)-1,1′-biphenyl-4- yl]carbonyl}-N-methyl-L-alanine |
| 201 | 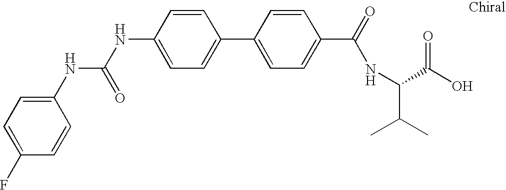 | 459.2 | 2.98 | 2 | S | N-{[4′-({[(4-fluorophenyl)- amino]carbonyl}amino)-1,1′-biphenyl-4- yl]carbonyl}-L-valine |
| 202 | 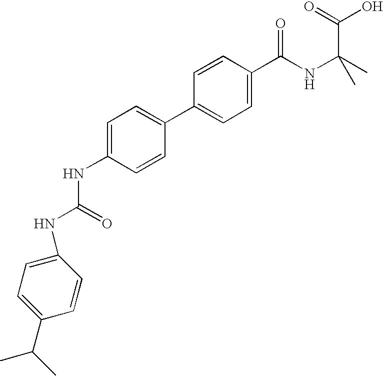 | 460.5 | 3.37 | 2 | not chiral | N-{[4′-({[(4-isopropylphenyl)- amino]carbonyl}amino)biphenyl-4- yl]carbonyl}-2-methylalanine |
| 203 | 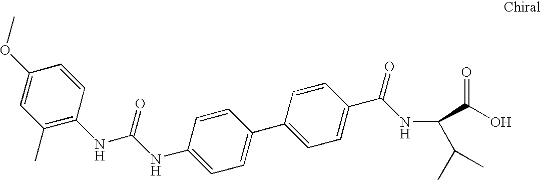 | 476.2 | 2.92 | 2 | R | N-{[4′-({[(4-methoxy-2-methylphenyl)- amino]carbonyl}amino)-1,1′-biphenyl-4- yl]carbonyl}-D-valine |
| 204 | 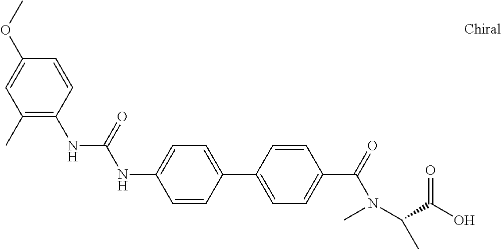 | 462.2 | 2.12 | 2 | S | N-{(4-({[(4-methoxy-2-methylphenyl)- amino]carbonyl}amino)-1,1′-biphenyl-4- yl]carbonyl}-N-methyl-L-alanine |
| 205 | 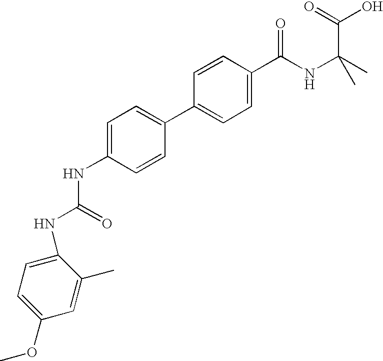 | 462.4 | 3.05 | 2 | not chiral | N-{[4′-({[(4-methoxy-2-methylphenyl)- amino]carbonyl}amino)biphenyl-4- yl]carbonyl}-2-methylalanine |
| 206 | 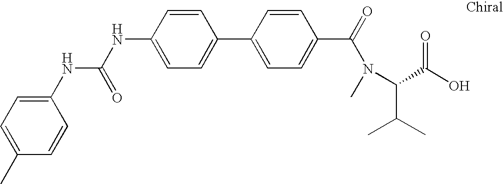 | 460.2 | 3.09 | 1 | S | N-{[4′-({[(4-methylphenyl)- amino]carbonyl}amino)-1,1′-biphenyl-4- yl]carbonyl}-N-methyl-L-valine |
| 207 | 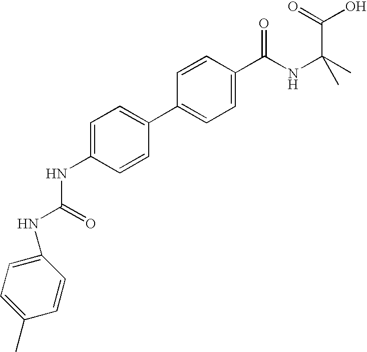 | 432.4 | 3.10 | 2 | not chiral | N-{[4′-({[(4-methylphenyl)- amino]carbonyl}amino)biphenyl-4- yl]carbonyl}-2-methylalanine |
| 208 | 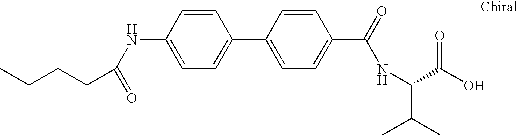 | 397.2 | 2.78 | 2 | S | N-{[4′-(pentanoylamino)-1,1′-biphenyl-4- yl]carbonyl}-L-valine |
| 209 | 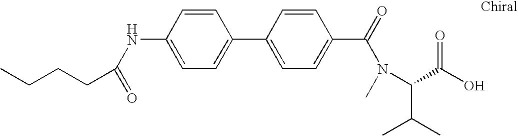 | 411.2 | 2.75 | 2 | S | N-methyl-N-{[4′-(pentanoylamino)1,1′- biphenyl-4-yl]carbonyl}-N-methyl-L-valine |

TABLE 2

| | | LC-MS | LC-MS | | | |
|---|---|---|---|---|---|---|
| Example | | MH+ | ret. time | LC-MS | | |
| No. | structure | (m/z) | (min) | method | chirality | IUPAC name |
| 210 | 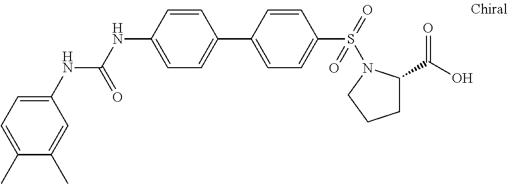 | 494.2 | 3.41 | 2 | S | 1-{[4′-({[(3,4-dimethylphenyl)- amino]carbonyl}amino)-1,1′-biphenyl-4- yl]sulfonyl}-L-proline |
| 211 | 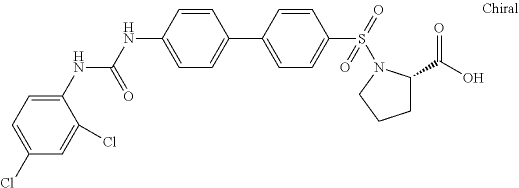 | 534.1 | 3.51 | 2 | S | 1-{[4′-({[(2,4-dichlorophenyl)- amino]carbonyl}amino)-1,1′-biphenyl-4- yl]sulfonyl}-L-proline |
| 212 | 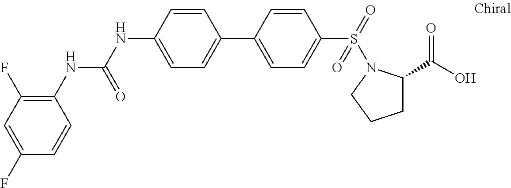 | 502.1 | 3.23 | 2 | S | 1-{[4′-({[(2,4-difluorophenyl)- amino]carbonyl}amino)-1,1′-biphenyl-4- yl]sulfonyl}-L-proline |
| 213 | 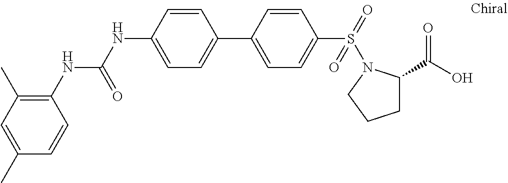 | 494.2 | 3.07 | 2 | S | 1-{[4′-({[(2,4-dimethylphenyl)- amino]carbonyl}amino)-1,1′-biphenyl-4- yl]sulfonyl}-L-proline |
| 214 | 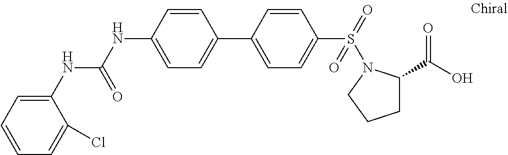 | 500.1 | 3.40 | 2 | S | 1-{[4′-({[(2-chlorophenyl)- amino]carbonyl}amino)-1,1′-biphenyl-4- yl]sulfonyl}-L-proline |
| 215 | 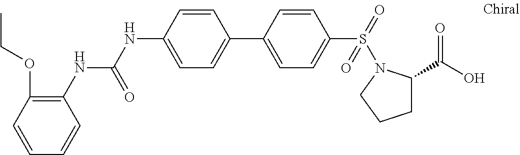 | 510.2 | 2.97 | 2 | S | 1-{[4′-({[(2-ethoxyphenyl)- amino]carbonyl}amino)-1,1′-biphenyl-4- yl]sulfonyl}-L-proline |
| 216 | 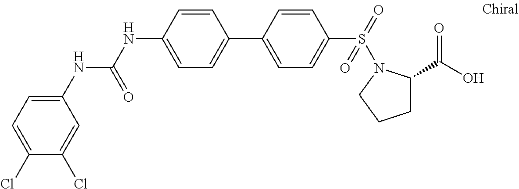 | 534.1 | 3.63 | 2 | S | 1-{[4′-({[(3,4-dichlorophenyl)- amino]carbonyl}amino)-1,1′-biphenyl-4- yl]sulfonyl}-L-proline |
| 217 | 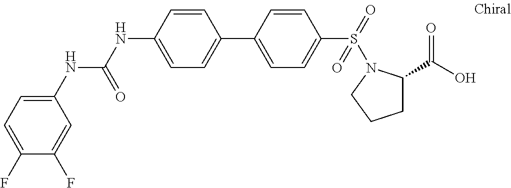 | 502.1 | 2.98 | 2 | S | 1-{[4′-({[(3,4-difluorophenyl)- amino]carbonyl}amino)-1,1′-biphenyl-4- yl]sulfonyl}-L-proline |
| 218 | 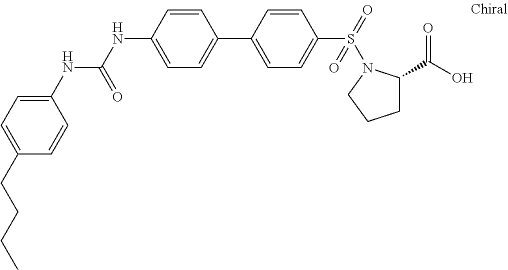 | 522.2 | 3.81 | 2 | S | 1-{[4′-({[(4-butylphenyl)- amino]carbonyl}amino)-1,1′-biphenyl-4- yl]sulfonyl}-L-proline |
| 219 | 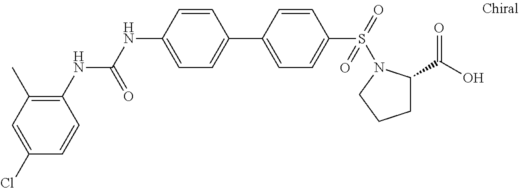 | 514.1 | 3.48 | 2 | S | 1-{[4′-({[(4-chloro-2-methylphenyl)- amino]carbonyl}amino)-1,1′-biphenyl-4- yl]sulfonyl}-L-proline |
| 220 | 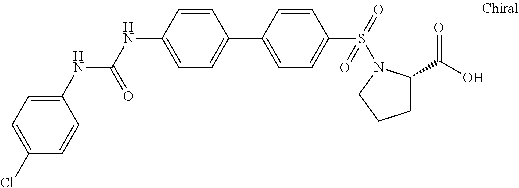 | 500.1 | 3.45 | 2 | S | 1-{[4′-({[(4-chlorophenyl)- amino]carbonyl}amino)-1,1′-biphenyl-4- yl]sulfonyl}-L-proline |
| 221 | 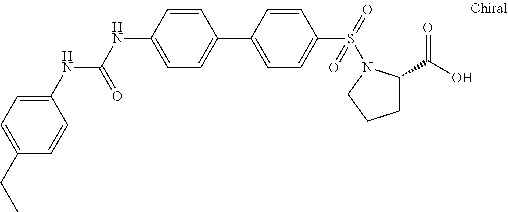 | 494.2 | 3.43 | 2 | S | 1-{[4′-({[(4-ethylphenyl)- amino]carbonyl}amino)-1,1′-biphenyl-4- yl]sulfonyl}-L-proline |
| 222 | 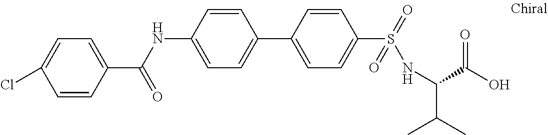 | 487.1 | 3.29 | 2 | S | N-({4′-[(4-chlorobenzoyl)amino]-1,1′- biphenyl-4-yl}sulfonyl)-L-valine |
| 223 | 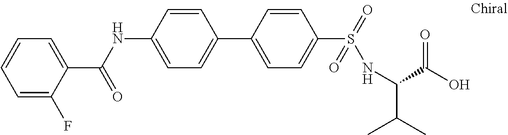 | 471.1 | 3.10 | 2 | S | N-({4′-[(2-fluorobenzoyl)amino]-1,1′- biphenyl-4-yl}sulfonyl)-L-valine |
| 224 | 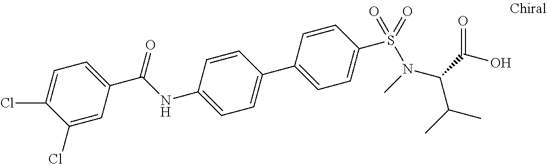 | 535.1 | 3.62 | 1 | S | N-({4′-[(3,4-dichlorobenzoyl)amino]- 1,1′-biphenyl-4-yl}sulfonyl)-N-methyl-L- valine |
| 225 | 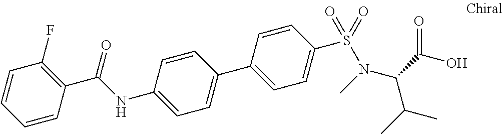 | 485.1 | 3.10 | 2 | S | N-({4′-[(2-fluorobenzoyl)amino]-1,1′- biphenyl-4-yl}sulfonyl)-N-methyl-L- valine |
| 226 | 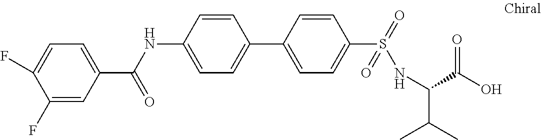 | 489.1 | 3.16 | 2 | S | N-({4′-[(3,4-difluorobenzoyl)amino]-1,1′- biphenyl-4-yl}sulfonyl)-L-valine |
| 227 | 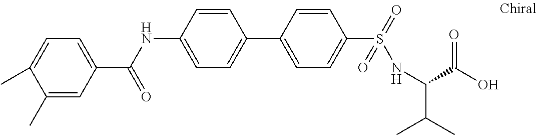 | 481.2 | 3.40 | 2 | S | N-({4′-[(3,4-dimethylbenzoyl)amino]- 1,1′-biphenyl-4-yl}sulfonyl)-L-valine |
| 228 | 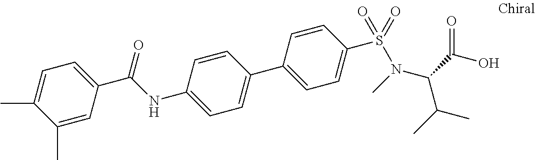 | 495.2 | 3.51 | 2 | S | N-({4′-[(3,4-dimethylbenzoyl)amino]- 1,1′-biphenyl-4-yl}sulfonyl)-N-methyl-L- valine |
| 229 | 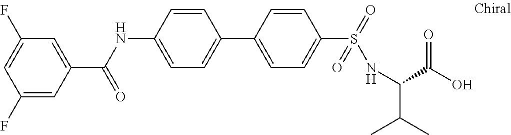 | 489.1 | 3.16 | 2 | S | N-({4′-[(3,5-difluorobenzoyl)amino]-1,1′- biphenyl-4-yl}sulfonyl)-L-valine |
| 230 | 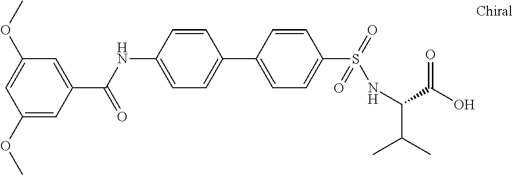 | 513.2 | 2.93 | 2 | S | N-({4′-[(3,5-dimethoxybenzoyl)amino]- 1,1′-biphenyl-4-yl}sulfonyl)-L-valine |
| 231 | 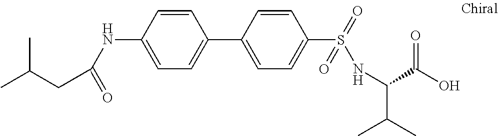 | 433.2 | 4.40 | 2 | S | N-({4′-[(3-methylbutanoyl)amino]-1,1′- biphenyl-4-yl}sulfonyl)-L-valine |
| 232 | 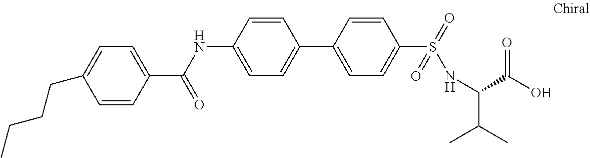 | 509.2 | 3.61 | 2 | S | N-({4′-[(4-butylbenzoyl)amino]-1,1′- biphenyl-4-yl}sulfonyl)-L-valine |
| 233 | 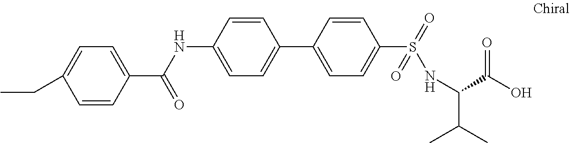 | 481.2 | 3.33 | 2 | S | N-({4′-[(4-ethyllbenzoyl)amino]-1,1′- biphenyl-4-yl}sulfonyl)-L-valine |
| 234 | 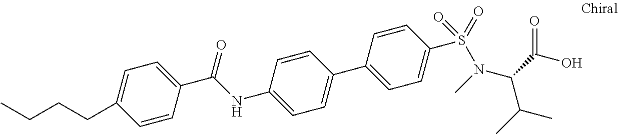 | 523.2 | 3.57 | 2 | S | N-({4′-[(4-butylbenzoyl)amino]-1,1′- biphenyl-4-yl}sulfonyl)-N-methyl-L- valine |
| 235 | 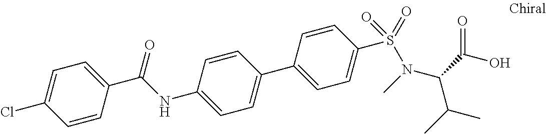 | 502.1 | 3.47 | 2 | S | N-({4′-[(4-chlorobenzoyl)amino]-1,1′- biphenyl-4-yl}sulfonyl)-N-methyl-L- valine |
| 236 | 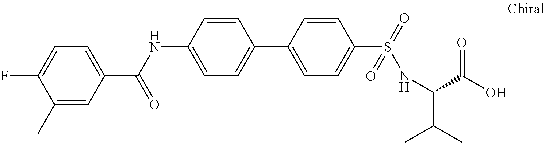 | 485.2 | 3.25 | 2 | S | N-({4′-[(4-fluoro-3- methylbenzoyl)amino]-1,1′-biphenyl-4- yl}sulfonyl)-L-valine |
| 237 | 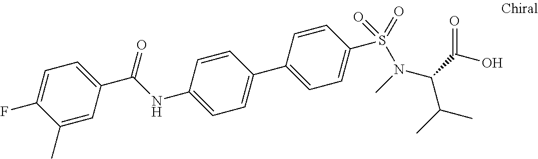 | 499.2 | 3.40 | 2 | S | N-({4′-[(4-fluoro-3- methylbenzoyl)amino]-1,1′-biphenyl-4- yl}sulfonyl)-N-methyl-L-valine |
| 238 | 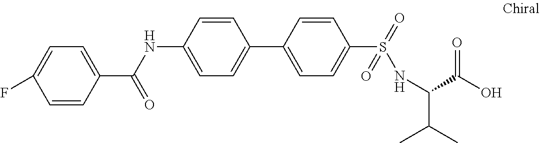 | 471.1 | 3.14 | 2 | S | N-({4′-[(4-fluorobenzoyl)amino]-1,1′- biphenyl-4-yl}sulfonyl)-L-valine |
| 239 | 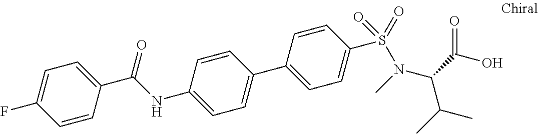 | 485.1 | 3.25 | 2 | S | N-({4′-[(4-fluorobenzoyl)amino]-1,1′- biphenyl-4-yl}sulfonyl)-N-methyl-L- valine |
| 240 | 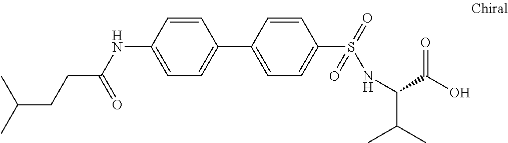 | 447.2 | 4.28 | 2 | S | N-({4′-[(4-methylpentanoyl)amino]-1,1′- biphenyl-4-yl}sulfonyl)-L-valine |
| 241 | 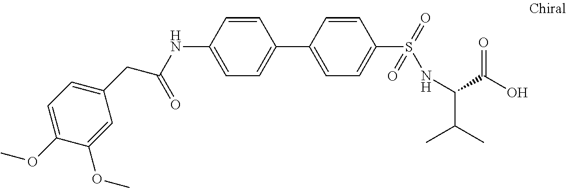 | 527.2 | 2.96 | 2 | S | N-[(4′-{[(3,4-dimethoxyphenyl)- acetyl]amino}-1,1′-biphenyl-4- yl)sulfonyl]-L-valine |
| 242 | 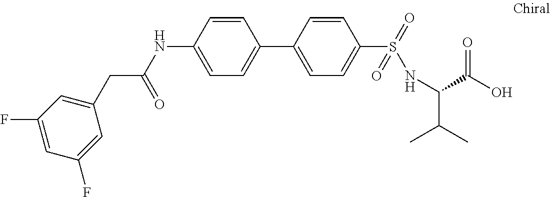 | 503.1 | 3.18 | 2 | S | N-[(4′-{[(3,5-difluorophenyl)- acetyl]amino}-1,1′-biphenyl-4- yl)sulfonyl]-L-valine |
| 243 | 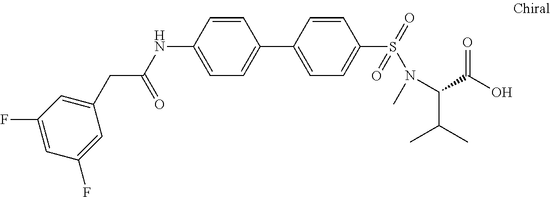 | 517.1 | 3.31 | 2 | S | N-[(4′-{[(3,5-difluorophenyl)- acetyl]amino}-1,1′-biphenyl-4- yl)sulfonyl]-N-methyl-L-valine |
| 244 | 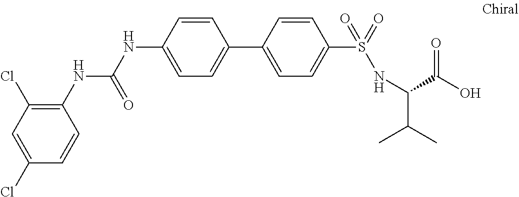 | 536.1 | 3.14 | 2 | S | N-{[4′-({[(2,4-dichlorophenyl)- amino]carbonyl}amino)-1,1′-biphenyl-4- yl]sulfonyl}-L-valine |
| 245 | 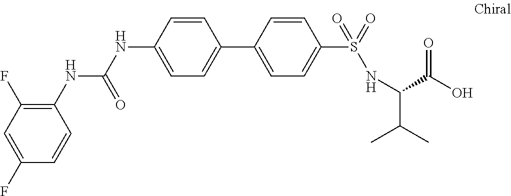 | 504.1 | 2.85 | 2 | S | N-{[4′-({[(2,4-difluorophenyl)- amino]carbonyl}amino)-1,1′-biphenyl-4- yl]sulfonyl}-L-valine |
| 246 | 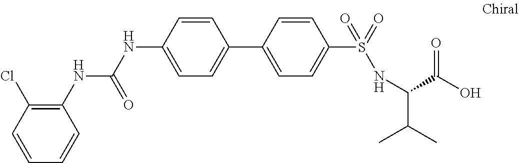 | 502.1 | 2.92 | 2 | S | N-{[4′-({[(2-chlorophenyl)- amino]carbonyl}amino)-1,1′-biphenyl-4- yl]sulfonyl}-L-valine |
| 247 | 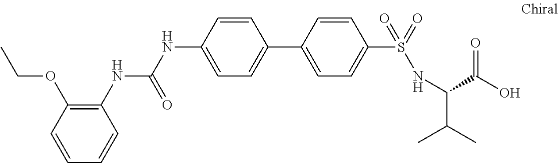 | 512.2 | 2.96 | 2 | S | N-{[4′-({[(2-ethoxyphenyl)- amino]carbonyl}amino)-1,1′-biphenyl-4- yl]sulfonyl}-L-valine |
| 248 | 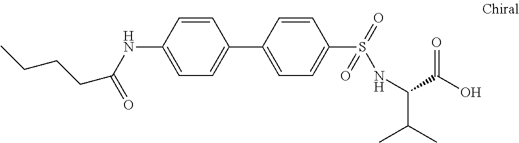 | 433.2 | 3.01 | 2 | S | N-{[4′-(pentanoylamino)-1,1′-biphenyl-4 yl]sulfonyl}-L-valine |
| 249 | 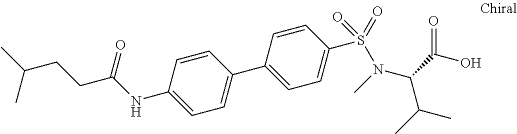 | 461.2 | 3.30 | 2 | S | N-methyl-N-({4′-[(4- methylpentanoyl)amino]-1,1′-biphenyl- 4-yl}sulfonyl)-L-valine |

By using the methods described above and by selecting the appropriate staring materials, additional compounds of Formula (I) can be prepared, such as those illustrated in Table 3 below.

TABLE 3

| Example | |
|---|---|
| No. | Structure |
| 250 | 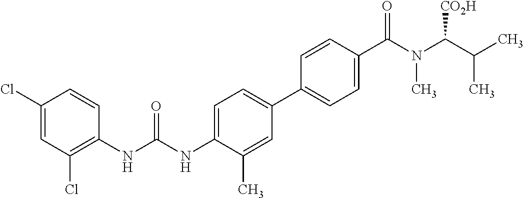 |
| 251 | 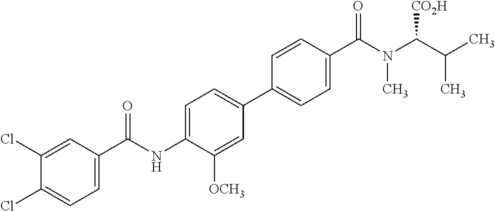 |
| 252 | 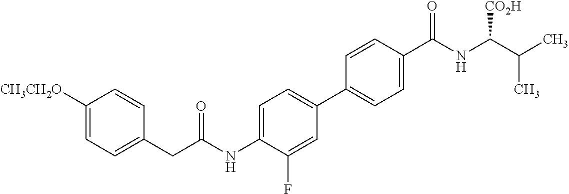 |
| 253 | 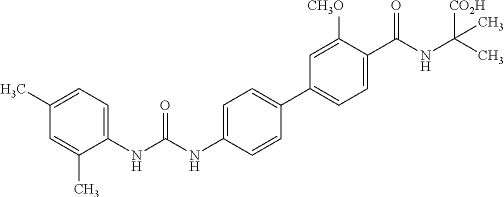 |
| 254 | 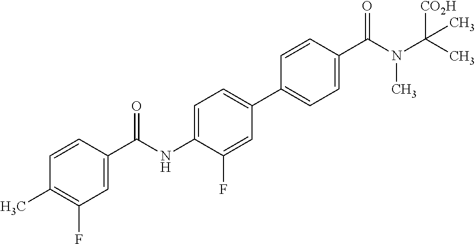 |
| 255 | 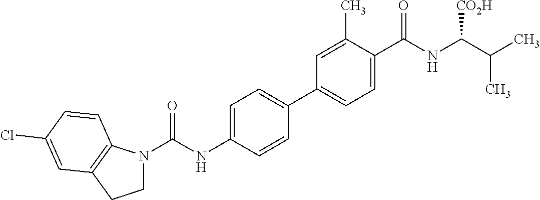 |
| 256 | 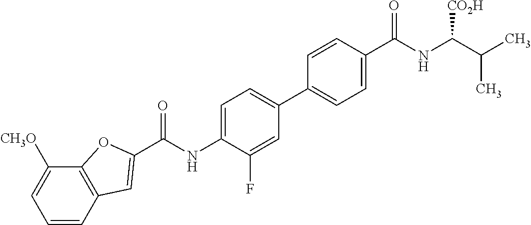 |
| 257 | 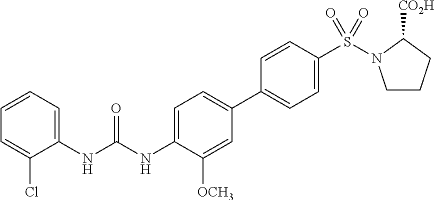 |
| 258 | 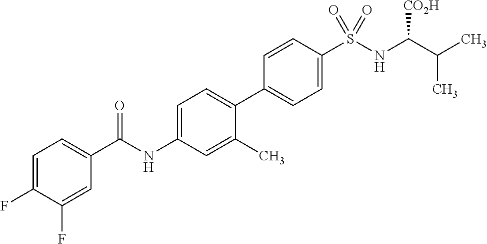 |
| 259 | 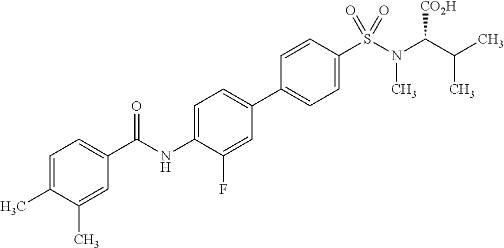 |
| 260 | 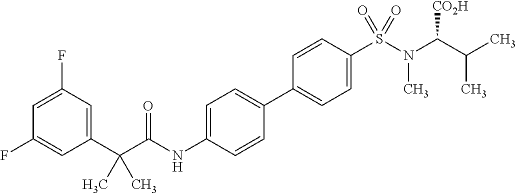 |

### Methods of Use

As used herein, various terms are defined below.

When introducing elements of the present invention or the preferred embodiment(s) thereof, the articles “a,” “an,” “the,” and “said” are intended to mean that there are one or more of the elements. The terms “comprising,” “including,” and “having” are intended to be inclusive and mean that there may be additional elements other than the listed elements.

The term “subject” as used herein includes mammals (e.g., humans and animals).

The term “treatment” includes any process, action, application, therapy, or the like, wherein a subject, including a human being, is provided medical aid with the object of improving the subject's condition, directly or indirectly, or slowing the progression of a condition or disorder in the subject.

The term “combination therapy” or “co-therapy” means the administration of two or more therapeutic agents to treat an obese condition and/or disorder. Such administration encompasses co-administration of two or more therapeutic agents in a substantially simultaneous manner, such as in a single capsule having a fixed ratio of active ingredients or in multiple, separate capsules for each inhibitor agent. In addition, such administration encompasses use of each type of therapeutic agent in a sequential manner.

The phrase “therapeutically effective” means the amount of each agent administered that will achieve the goal of improvement in an obese condition or disorder severity, while avoiding or minimizing adverse side effects associated with the given therapeutic treatment.

The term “pharmaceutically acceptable” means that the subject item is appropriate for use in a pharmaceutical product.

The compounds of Formula (I) of this invention are expected to be valuable as therapeutic agents. Accordingly, an embodiment of this invention includes a method of treating the various conditions in a patient (including mammals) which comprises administering to said patient a composition containing an amount of the compound of Formula (I) that is effective in treating the target condition.

An object of this invention is to provide methods for treating obesity and inducing weight loss in an individual by administration of a compound of the invention. The method of the invention comprises administering to an individual a therapeutically effective amount of at least one compound of the invention, or a prodrug thereof, which is sufficient to induce weight loss. The invention further comprises a method of preventing weight gain in an individual by administering an amount of at least one compound of the invention, or a prodrug thereof, which is sufficient to prevent weight gain.

The present invention also relates to the use of the compounds of this invention for the treatment of obesity-related diseases including associated dyslipidemia and other obesity- and overweight-related complications such as, for example, cholesterol gallstones, gallbladder disease, gout, cancer (e.g., colon, rectum, prostate, breast, ovary, endometrium, cervix, gallbladder, and bile duct), menstrual abnormalities, infertility, polycystic ovaries, osteoarthritis, and sleep apnea, as well as for a number of other pharmaceutical uses associated therewith, such as the regulation of appetite and food intake, dyslipidemia, hypertriglyceridemia, Syndrome X, type 2 diabetes (non-insulin-dependent diabetes), atherosclerotic diseases such as heart failure, hyperlipidemia, hypercholesteremia, low HDL levels, hypertension, cardiovascular disease (including atherosclerosis, coronary heart disease, coronary artery disease, and hypertension), cerebrovascular disease such as stroke, and peripheral vessel disease. The compounds of this invention may also be useful for treating physiological disorders related to, for example, regulation of insulin sensitivity, inflammatory response, plasma triglycerides, HDL, LDL and cholesterol levels and the like.

Compounds of Formula (I) may be administered alone or in combination with one or more additional therapeutic agents. Combination therapy includes administration of a single pharmaceutical dosage formulation which contains a compound of Formula (I) and one or more additional therapeutic agents, as well as administration of the compound of Formula (I) and each additional therapeutic agents in its own separate pharmaceutical dosage formulation. For example, a compound of Formula (I) and a therapeutic agent may be administered to the patient together in a single oral dosage composition such as a tablet or capsule, or each agent may be administered in separate oral dosage formulations.

Where separate dosage formulations are used, the compound of Formula (I) and one or more additional therapeutic agents may be administered at essentially the same time (e.g., concurrently) or at separately staggered times (e.g., sequentially).

For example, the compound of Formula (I) may be used in combination with other therapies and drugs useful for the treatment of obesity and diabetes. For example, anti-obesity drugs include β-3 agonists such as CL 316,243; cannabinoid (e.g., CB-1) antagonists, such as, for example, rimonabant (Acomplia); neuropeptide Y5 inhibitors; appetite suppressants, such as, for example, sibutramine (Meridia); and lipase inhibitors, such as, for example, orlistat (Xenical). The compounds of the present invention may also be administered in combination with a drug compound that modulates digestion and/or metabolism such as drugs that modulate thermogenesis, lipolysis, gut motility, fat absorption, and satiety.

In addition, the compounds of Formula (I) may be administered in combination with one or more of the following agents for the treatment of diabetes or diabetes-related disorders including PPAR ligands (agonists, antagonists), insulin secretagogues, for example, sulfonylurea drugs and non-sulfonylurea secretagogues, oglucosidase inhibitors, insulin sensitizers, hepatic glucose output lowering compounds, and insulin and insulin derivatives. Such therapies may be administered prior to, concurrently with, or following administration of the compounds of the invention. Insulin and insulin derivatives include both long and short acting forms and formulations of insulin. PPAR ligands may include agonists and/or antagonists of any of the PPAR receptors or combinations thereof. For example, PPAR ligands may include ligands of PPAR-α, PPAR-γ, PPAR-δ or any combination of two or three of the receptors of PPAR. PPAR ligands include, for example, rosiglitazone, troglitazone, and pioglitazone. Sulfonylurea drugs include, for example, glyburide, glimepiride, chlorpropamide, tolbutamide, and glipizide. α-glucosidase inhibitors that may be useful in treating diabetes when administered with a compound of the invention include acarbose, miglitol, and voglibose. Insulin sensitizers that may be useful in treating diabetes include PPAR-γ agonists such as the glitazones (e.g., troglitazone, pioglitazone, englitazone, MCC-555, rosiglitazone, and the like) and other thiazolidinedione and non-thiazolidinedione compounds; biguanides such as metformin and phenformin; protein tyrosine phosphatase-1B (PIP-1B) inhibitors; dipeptidyl peptidase IV (DPP-IV) inhibitors, and 11beta-HSD inhibitors. Hepatic glucose output lowering compounds that may be useful in treating diabetes when administered with a compound of the invention include glucagon anatgonists and metformin, such as Glucophage and Glucophage XR. Insulin secretagogues that may be useful in treating diabetes when administered with a compound of the invention include sulfonylurea and non-sulfonylurea drugs: GLP-1, GIP, PACAP, secretin, and derivatives thereof; nateglinide, meglitinide, repaglinide, glibenclamide, glimepiride, chlorpropamide, glipizide. GLP-1 includes derivatives of GLP-1 with longer half-lives than native GLP-1, such as, for example, fatty-acid derivatized GLP-1 and exendin.

Compounds of the invention may also be used in methods of the invention in combination with drugs commonly used to treat lipid disorders in patients. Such drugs include, but are not limited to, HMG-CoA reductase inhibitors, nicotinic acid, fatty acid lowering compounds (e.g., acipimox); lipid lowering drugs (e.g., stanol esters, sterol glycosides such as tiqueside, and azetidinones such as ezetimibe), ACAT inhibitors (such as avasimibe), bile acid sequestrants, bile acid reuptake inhibitors, microsomal triglyceride transport inhibitors, and fibric acid derivatives. HMG-CoA reductase inhibitors include, for example, lovastatin, simvastatin, pravastatin, fluvastatin, atorvastatin, rivastatin, itavastatin, cerivastatin, and ZD-4522. Fibric acid derivatives include, for example, clofibrate, fenofibrate, bezafibrate, ciprofibrate, beclofibrate, etofibrate, and gemfibrozil. Sequestrants include, for example, cholestyramine, colestipol, and dialkylaminoalkyl derivatives of a cross-linked dextran.

Compounds of the invention may also be used in combination with anti-hypertensive drugs, such as, for example, β-blockers and ACE inhibitors. Examples of additional anti-hypertensive agents for use in combination with the compounds of the present invention include calcium channel blockers (L-type and T-type; e.g., diltiazem, verapamil, nifedipine, amlodipine and mybefradil), diuretics (e.g., chlorothiazide, hydrochlorothiazide, flumethiazide, hydroflumethiazide, bendroflumethiazide, methylchlorothiazide, trichloromethiazide, polythiazide, benzthiazide, ethacrynic acid tricrynafen, chlorthalidone, furosemide, musolimine, bumetanide, triamtrenene, amiloride, spironolactone), renin inhibitors, ACE inhibitors (e.g., captopril, zofenopril, fosinopril, enalapril, ceranopril, cilazopril, delapril, pentopril, quinapril, ranipril, lisinopril), AT-1 receptor antagonists (e.g., losartan, irbesartan, valsartan), ET receptor antagonists (e.g., sitaxsentan, atrsentan, neutral endopeptidase (NEP) inhibitors, vasopepsidase inhibitors (dual NEP-ACE inhibitors) (e.g., omapatrilat and gemopatrilat), and nitrates.

The compounds of Formula (I) may also be utilized, in free base form or in compositions, as well as in research and diagnostics or as analytical reference standards, and the like, which are well known in the art. Therefore, the present invention includes compositions which are comprised of an inert carrier and an effective amount of a compound of Formula (I) or a salt, or ester thereof. An inert carrier is any material which does not interact with the compound to be carried and which lends support, means of conveyance, bulk, traceable material, and the like to the compound to be carried. An effective amount of the compound is that amount which produces a result or exerts an influence on the particular procedure being performed.

It is anticipated that prodrug forms of the compounds of this invention will prove useful in certain circumstances, and such compounds are also intended to fall within the scope of the invention. Prodrug forms may have advantages over the parent compounds exemplified herein, in that they are better absorbed, better distributed, more readily penetrate the central nervous system, are more slowly metabolized or cleared, etc. Prodrug forms may also have formulation advantages in terms of crystallinity or water solubility. For example, compounds of the invention having one or more hydroxyl groups may be converted to esters or carbonates bearing one or more carboxyl, hydroxyl or amino groups, which are hydrolyzed at physiological pH values or are cleaved by endogenous esterases or lipases in vivo (see, e.g., U.S. Pat. Nos. 4,942,184; 4,960,790; 5,817,840; and 5,824,701, all of which are incorporated herein by reference in their entirety, and references therein).

### Pharmaceutical Compositions

Based on the above tests, or other well known assays used to determine the efficacy for treatment of conditions identified above in mammals, and by comparison of these results with the results of known medicaments that are used to treat these conditions, the effective dosage of the compounds of this invention can readily be determined for treatment of each desired indication. The amount of the active ingredient to be administered in the treatment of one of these conditions can vary widely according to such considerations as the particular compound and dosage unit employed, the mode of administration, the period of treatment, the age and sex of the patient treated, and the nature and extent of the condition treated.

The total amount of the active ingredient to be administered may generally range from about 0.001 mg/kg to about 200 mg/kg, and preferably from about 0.01 mg/kg to about 200 mg/kg body weight per day. A unit dosage may contain from about 0.05 mg to about 1500 mg of active ingredient, and may be administered one or more times per day. The daily dosage for administration by injection, including intravenous, intramuscular, subcutaneous, and parenteral injections, and use of infusion techniques may be from about 0.01 to about 200 mg/kg. The daily rectal dosage regimen may be from 0.01 to 200 mg/kg of total body weight. The transdermal concentration may be that required to maintain a daily dose of from 0.01 to 200 mg/kg.

Of course, the specific initial and continuing dosage regimen for each patient will vary according to the nature and severity of the condition as determined by the attending diagnostician, the activity of the specific compound employed, the age of the patient, the diet of the patient, time of administration, route of administration, rate of excretion of the drug, drug combinations, and the like. The desired mode of treatment and number of doses of a compound of the present invention or a pharmaceutically acceptable salt thereof may be ascertained by those skilled in the art using conventional treatment tests.

The compounds of this invention may be utilized to achieve the desired pharmacological effect by administration to a subject in need thereof in an appropriately formulated pharmaceutical composition. A subject, for example, may be a mammal, including a human, in need of treatment for a particular condition or disease. Therefore, the present invention includes pharmaceutical compositions which are comprised of a pharmaceutically acceptable carrier and a pharmaceutically effective amount of a compound identified by the methods described herein, or a pharmaceutically acceptable salt or ester thereof. A pharmaceutically acceptable carrier is any carrier which is relatively non-toxic and innocuous to a patient at concentrations consistent with effective activity of the active ingredient so that any side effects ascribable to the carrier do not vitiate the beneficial effects of the active ingredient. A pharmaceutically effective amount of a compound is that amount which produces a result or exerts an influence on the particular condition being treated. The compounds identified by the methods described herein may be administered with a pharmaceutically-acceptable carrier using any effective conventional dosage unit forms, including, for example, immediate and timed release preparations, orally, parenterally, topically, or the like.

For oral administration, the compounds may be formulated into solid or liquid preparations such as, for example, capsules, pills, tablets, troches, lozenges, melts, powders, solutions, suspensions, or emulsions, and may be prepared according to methods known to the art for the manufacture of pharmaceutical compositions. The solid unit dosage forms may be a capsule which can be of the ordinary hard- or soft-shelled gelatin type containing, for example, surfactants, lubricants, and inert fillers such as lactose, sucrose, calcium phosphate, and corn starch

In another embodiment, the compounds of this invention may be tableted with conventional tablet bases such as lactose, sucrose, and cornstarch in combination with binders such as acacia, cornstarch, or gelatin; disintegrating agents intended to assist the break-up and dissolution of the tablet following administration such as potato starch, alginic acid, corn starch, and guar gum; lubricants intended to improve the flow of tablet granulation and to prevent the adhesion of tablet material to the surfaces of the tablet dies and punches, for example, talc, stearic acid, or magnesium, calcium or zinc stearate; dyes; coloring agents; and flavoring agents intended to enhance the aesthetic qualities of the tablets and make them more acceptable to the patient. Suitable excipients for use in oral liquid dosage forms include diluents such as water and alcohols, for example, ethanol, benzyl alcohol, and polyethylene alcohols, either with or without the addition of a pharmaceutically acceptable surfactant, suspending agent, or emulsifying agent. Various other materials may be present as coatings or to otherwise modify the physical form of the dosage unit. For instance tablets, pills or capsules may be coated with shellac, sugar or both.

Dispersible powders and granules are suitable for the preparation of an aqueous suspension. They provide the active ingredient in admixture with a dispersing or wetting agent, a suspending agent, and one or more preservatives. Suitable dispersing or wetting agents and suspending agents are exemplified by those already mentioned above. Additional excipients, for example, those sweetening, flavoring and coloring agents described above, may also be present.

The pharmaceutical compositions of this invention may also be in the form of oil-in-water emulsions. The oily phase may be a vegetable oil such as liquid paraffin or a mixture of vegetable oils. Suitable emulsifying agents may be (1) naturally occurring gums such as gum acacia and gum tragacanth, (2) naturally occurring phosphatides such as soy bean and lecithin, (3) esters or partial esters derived from fatty acids and hexitol anhydrides, for example, sorbitan monooleate, and (4) condensation products of said partial esters with ethylene oxide, for example, polyoxyethylene sorbitan monooleate. The emulsions may also contain sweetening and flavoring agents.

Oily suspensions may be formulated by suspending the active ingredient in a vegetable oil such as, for example, arachis oil, olive oil, sesame oil, or coconut oil; or in a mineral oil such as liquid paraffin. The oily suspensions may contain a thickening agent such as, for example, beeswax, hard paraffin, or cetyl alcohol. The suspensions may also contain one or more preservatives, for example, ethyl or n-propyl p-hydroxybenzoate; one or more coloring agents; one or more flavoring agents; and one or more sweetening agents such as sucrose or saccharin.

Syrups and elixirs may be formulated with sweetening agents such as, for example, glycerol, propylene glycol, sorbitol, or sucrose. Such formulations may also contain a demulcent, and preservative, flavoring and coloring agents.

The compounds of this invention may also be administered parenterally, that is, subcutaneously, intravenously, intramuscularly, or interperitoneally, as injectable dosages of the compound in a physiologically acceptable diluent with a pharmaceutical carrier which may be a sterile liquid or mixture of liquids such as water, saline, aqueous dextrose and related sugar solutions; an alcohol such as ethanol, isopropanol, or hexadecyl alcohol; glycols such as propylene glycol or polyethylene glycol; glycerol ketals such as 2,2-dimethyl-1,1-dioxolane-4-methanol, ethers such as poly(ethyleneglycol) 400; an oil; a fatty acid; a fatty acid ester or glyceride; or an acetylated fatty acid glyceride with or without the addition of a pharmaceutically acceptable surfactant such as a soap or a detergent, suspending agent such as pectin, carbomers, methylcellulose, hydroxypropylmethyl-cellulose, or carboxymethylcellulose, or emulsifying agent and other pharmaceutical adjuvants.

Illustrative of oils which can be used in the parenteral formulations of this invention are those of petroleum, animal, vegetable, or synthetic origin, for example, peanut oil, soybean oil, sesame oil, cottonseed oil, corn oil, olive oil, petrolatum, and mineral oil. Suitable fatty acids include oleic acid, stearic acid, and isostearic acid. Suitable fatty acid esters are, for example, ethyl oleate and isopropyl myristate. Suitable soaps include fatty alkali metal, ammonium, and triethanolamine salts and suitable detergents include cationic detergents, for example, dimethyl dialkyl ammonium halides, alkyl pyridinium halides, and alkylamine acetates; anionic detergents, for example, alkyl, aryl, and olefin sulfonates, alkyl, olefin, ether, and monoglyceride sulfates, and sulfosuccinates; nonionic detergents, for example, fatty amine oxides, fatty acid alkanolamides, and polyoxyethylenepolypropylene copolymers; and amphoteric detergents, for example, allyl-beta-aminopropionates, and 2-alkyl-imidazoline quarternary ammonium salts, as well as mixtures.

The parenteral compositions of this invention may typically contain from about 0.5% to about 25% by weight of the active ingredient in solution. Preservatives and buffers may also be used advantageously. In order to minimize or eliminate irritation at the site of injection, such compositions may contain a non-ionic surfactant having a hydrophile-lipophile balance (HLB) of from about 12 to about 17. The quantity of surfactant in such formulation ranges from about 5% to about 15% by weight. The surfactant can be a single component having the above HLB or can be a mixture of two or more components having the desired HLB.

Illustrative of surfactants used in parenteral formulations are the class of polyethylene sorbitan fatty acid esters, for example, sorbitan monooleate and the high molecular weight adducts of ethylene oxide with a hydrophobic base, formed by the condensation of propylene oxide with propylene glycol.

The pharmaceutical compositions may be in the form of sterile injectable aqueous suspensions. Such suspensions may be formulated according to known methods using suitable dispersing or wetting agents and suspending agents such as, for example, sodium carboxymethylcellulose, methylcellulose, hydroxypropylmethyl-cellulose, sodium alginate, polyvinylpyrrolidone, gum tragacanth and gum acacia; dispersing or wetting agents which may be a naturally occurring phosphatide such as lecithin, a condensation product of an alkylene oxide with a fatty acid, for example, polyoxyethylene stearate, a condensation product of ethylene oxide with a long chain aliphatic alcohol, for example, heptadecaethyleneoxycetanol, a condensation product of ethylene oxide with a partial ester derived form a fatty acid and a hexitol such as polyoxyethylene sorbitol monooleate, or a condensation product of an ethylene oxide with a partial ester derived from a fatty acid and a hexitol anhydride, for example polyoxyethylene sorbitan monooleate.

The sterile injectable preparation may also be a sterile injectable solution or suspension in a non-toxic parenterally acceptable diluent or solvent. Diluents and solvents that may be employed are, for example, water, Ringer's solution, and isotonic sodium chloride solution. In addition, sterile fixed oils are conventionally employed as solvents or suspending media. For this purpose, any bland, fixed oil may be employed including synthetic mono or diglycerides. In addition, fatty acids such as oleic acid may be used in the preparation of injectables.

A composition of the invention may also be administered in the form of suppositories for rectal administration of the drug. These compositions may be prepared by mixing the drug with a suitable non-irritation excipient which is solid at ordinary temperatures but liquid at the rectal temperature and will therefore melt in the rectum to release the drug. Such material are, for example, cocoa butter and polyethylene glycol.

Another formulation employed in the methods of the present invention employs transdermal delivery devices (“patches”). Such transdermal patches may be used to provide continuous or discontinuous infusion of the compounds of the present invention in controlled amounts. The construction and use of transdermal patches for the delivery of pharmaceutical agents is well known in the art (see, e.g., U.S. Pat. No. 5,023,252, incorporated herein by reference). Such patches may be constructed for continuous, pulsatile, or on demand delivery of pharmaceutical agents.

Another formulation employs the use of biodegradable microspheres that allow controlled, sustained release of the compounds of this invention. Such formulations can be comprised of synthetic polymers or copolymers. Such formulations allow for injection, inhalation, nasal, or oral administration. The construction and use of biodegradable microspheres for the delivery of pharmaceutical agents is well known in the art (e.g., U.S. Pat. No. 6,706,289, incorporated herein by reference).

It may be desirable or necessary to introduce the pharmaceutical composition to the patient via a mechanical delivery device. The construction and use of mechanical delivery devices for the delivery of pharmaceutical agents is well known in the art. For example, direct techniques for administering a drug directly to the brain usually involve placement of a drug delivery catheter into the patient's ventricular system to bypass the blood-brain barrier. One such implantable delivery system, used for the transport of agents to specific anatomical regions of the body, is described in U.S. Pat. No. 5,011,472, incorporated herein by reference.

The compositions of the invention may also contain other conventional pharmaceutically acceptable compounding ingredients, generally referred to as carriers or diluents, as necessary or desired. Any of the compositions of this invention may be preserved by the addition of an antioxidant such as ascorbic acid or by other suitable preservatives. Conventional procedures for preparing such compositions in appropriate dosage forms can be utilized.

Commonly used pharmaceutical ingredients which may be used as appropriate to formulate the composition for its intended route of administration include: acidifying agents, for example, but are not limited to, acetic acid, citric acid; fumaric acid, hydrochloric acid, nitric acid; and alkalinizing agents such as, but are not limited to, ammonia solution, ammonium carbonate, diethanolamine, monoethanolamine, potassium hydroxide, sodium borate, sodium carbonate, sodium hydroxide, triethanolamine, trolamine.

The compounds identified by the methods described herein may be administered as the sole pharmaceutical agent or in combination with one or more other pharmaceutical agents where the combination causes no unacceptable adverse effects. For example, the compounds of this invention can be combined with known anti-obesity, or with known antidiabetic or other indication agents, and the like, as well as with admixtures and combinations thereof.

The compounds identified by the methods described herein may also be utilized, in free base form or in compositions, in research and diagnostics, or as analytical reference standards, and the like. Therefore, the present invention includes compositions which are comprised of an inert carrier and an effective amount of a compound identified by the methods described herein, or a salt or ester thereof. An inert carrier is any material which does not interact with the compound to be carried and which lends support, means of conveyance, bulk traceable material, and the like to the compound to be carried. An effective amount of compound is that amount which produces a result or exerts an influence on the particular procedure being performed.

Formulations suitable for subcutaneous, intravenous, intramuscular, and the like; suitable pharmaceutical carriers; and techniques for formulation and administration may be prepared by any of the methods well known in the art (see, e.g., Remington's Pharmaceutical Sciences, Mack Publishing Co., Easton, Pa., 20^{th} edition, 2000).

### Biological Activity of the Compounds

In order that this invention may be better understood, the following examples are set forth. These examples are for the purpose of illustration only, and are not to be construed as limiting the scope of the invention in any manner. All publications mentioned herein are incorporated by reference in their entirety.

Demonstration of the activity of the compounds of the present invention may be accomplished through in vitro, ex vivo, and in vivo assays that are well known in the art. For example, to demonstrate the efficacy of a pharmaceutical agent for the treatment of obesity and related disorders, the following assays may be used.

### Evaluation of Compound Effect on the Inhibition of DGAT-1 Enzyme Activity

The human DGAT-1 gene (see, e.g., U.S. Pat. No. 6,100,077) was isolated from a human cDNA library by PCR. Recombinant AcNPV baculovirus was constructed in which the gene for occlusion body forming protein polyhedrin was replaced with the DGAT-1 gene. The DGAT-1 gene sequence was inserted into the AcNPV genome 3′ to the polyhedrin promoter sequence placing DGAT-1 under the transcriptional control of the polyhedrin promoter. Spodoptera frugiperda-derived Sf9 insect cells were infected with DGAT-1-containing recombinant baculovirus at the multiplicity of infection of 5 and harvested 48 h post-infection. DGAT-1-expressing insect cells were homogenized in 10 mM Tris, 250 mM sucrose, pH 75 at the concentration of 100 mg of wet cell biomass per mL. The homogenate was centrifuged at 25,000 g for 30 minutes. The 25,000 g pellet was discarded and the supernatant was centrifuged at 100,000 g for 1 h. The 100,000 g supernatant was discarded and the 100,000 g DGAT-1-containing membrane pellet was re-suspended in 10 mM Tris, 50% (v/v) glycerol pH 7.5.

DGAT-1 enzyme activity was determined by a phase partitioning protocol. Specifically, DGAT-1 containing membranes were incubated in 20 μM didecanoyl glycerol 5 μM ¹⁴C-decanoyl-CoA, 2 mM MgCl₂, 0.04% BSA, 20 mM HEPES, pH 7.5 buffer in the presence of varying concentrations of inhibitors. Assays were performed in 100 μl volumes in 96-well microtiter plates 0.5 μg total membrane protein per well. The assay was initiated by substrate and mixed gently for 1 h at ambient temperature. Activity was quenched by the addition of 25 μl of 0.1% phosphoric acid solution. Selective extraction of the hydrophobic tridecanolyglycerol product was accomplished by the addition of 150 μl phase partitioning scintillation fluid Microscint® (Packard, Inc.) and vigorous mixing for 30 minutes. Quantification of the product was accomplished by a MicroBeta® scintillation counter (Wallac, Inc.) after settling for approximately 16 h at ambient temperatures.

### Evaluation of Compound Effect on the Inhibition of Cellular Triglyceride Deposition

The cell-based assay for DGAT-1 was conducted with human colorectal adenocarcinoma cells Hr-29 (HTB-38, ATCC). HT-29 cells were grown in 75 cm² plate until ˜90% confluent in DMEM media with 10% FBS, PSF, glutamine, and 10 mM acetate. Cells were then re-plated in 24-well plates to give 1:1.2 dilution and grown approximately 16 h. Triacylglyceride formation was stimulated by the addition of lauric acid to 0.01% final concentration in the presence of varying concentrations of inhibitors. After 6 h, cells were released from the plate by trypsin, collected by centrifugation, re-suspended in water, transferred to glass HPLC, frozen at −70° C., and lyophilized. Freeze dried cell pellets were re-suspended in 150 μl HPLC grade tetrahydrofuran and sealed in the vials. Vials were sonicated for 30 minutes with heating in a sonicating water bath (Fisher, Inc.). Cellular triacylglycerides were quantified by HPLC (HP1100, Agilent, Inc.) utilizing evaporative light-scattering detection (PL-ELS 1000, Polymer Labs, Inc.). Chromatographic separation was accomplished by 30 to 100% B buffer in 4 minutes followed by 3 minutes at 100% B buffer using a PLRP S 100 column (5 micron, 150×4.6 mm, Polymer Labs, Inc.) at 50° C. (A: 50% acetonitrile, 2.5% methanol, B: 100% tetrahydrofuran). Sample injections were 20 μl and the detector was set at 0.4 SLM, 40° C. nebulizer and 80° C. evaporator. Non-polar fatty acids and glycerol lipids were identified and quantified by using commercially available standards.

### Evaluation of Compound Efficacy on the Reduction of Body Weight in Diet-Induced Obese Mice

The purpose of this protocol is to determine the effect of chronic administration of a compound on the body weight of mice made obese by exposure to a 45% kcal/g high fat diet for more than 10 weeks. The body weight of mice selected for these studies was higher than three standard deviations from the weight of a control group of mice fed standard low fat (56% fat) mouse chow. Diet-induced obese (DIO) animals have been used frequently in the determination of compound efficacy in the reduction of body weight (see, e.g., Brown, et al., Brit. J. Pharmacol. 132:1898-1904, 2001; Guerre-Millo, et al., J. Biol. Chem. 275(22):16638-42, 2000; Han, et al., Intl. J. Obesity and Related Metabolic Disorders 23(2): 174-79, 1999; Surwit, et al., Endocrinol. 141(10):3630-37, 2000).

This animal model has been successfully used in the identification and characterization of the efficacy profile of compounds that are or have been used in the management of body weight in obese humans (see, e.g., Brown, et al., 2001; Guerre-Millo, et al., 2000; Han, et al., 1999).

A typical study included 60-80 male C57bl/J6 mice (n=10/treatment group) with an average body weight of approximately 45 g. Mice were kept in standard animal rooms under controlled temperature and humidity and a 12 hour/12 hour light/dark cycle. Water and food were continuously available. Mice were individually housed. Animals were sham dosed with study vehicle for at least four days before the recording of two-day baseline measurements of body weight and 24-hour food and water consumption. Mice were assigned to one of 6-8 treatment groups based upon their body weight on baseline. The groups were set up so that the mean and standard error of the mean of body weight were similar.

Animals were orally gavaged (5 mL/kg) daily before the dark phase of the light/dark cycle for a predetermined number of days (typically 8-14 days) with their assigned dose/compound. Body weight, and food and water consumption were measured. Data was analyzed using appropriate statistics following the research design. On the final day, animals were euthanized using CO₂ inhalation.

Compounds were typically dosed at 5 or 10 mg/kg p.o. q.d. as a suspension formulation in 50:50 PEG/water, or p.o. b.i.d. as a suspension formulation in 0.5% methylcellulose, and compounds were considered to be active if a statistically significant reduction in body weight was observed for the treated animals after a treatment period of at least seven days, relative to vehicle-treated control animals.

The structures, materials, compositions, and methods described herein are intended to be representative examples of the invention, and it will be understood that the scope of the invention is not limited by the scope of the examples. Those skilled in the art will recognize that the invention may be practiced with variations on the disclosed structures, materials, compositions and methods, and such variations are regarded as within the ambit of the invention.

What is claimed:

1. A compound of Formula (I) 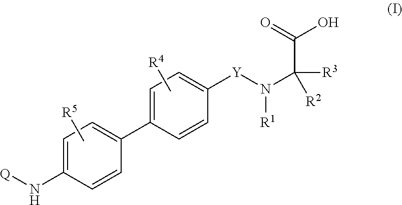
wherein
Y is C═O or S(═O)₂;
R¹ is hydrogen or (C₁-C₆)alkyl;
R² is (C₁-C₆)alkyl, hydroxy-C₁₋₆)alkyl, (C₁-C₆)alkoxy-(C₁-C₆)alkyl, amino-(C₁-C₆)alkyl, (C₁-C₆)alkylamino-(C₁-C₆)alkyl, or bis[(C₁-C₆)alkyl]amino-(C₁-C₆)alkyl;
R³ is hydrogen; or
R¹ is hydrogen or (C₁-C₆)alkyl;
R² is R⁶(CH₂)ₘ,
wherein
m is 0 to 3,
R⁶ is phenyl optionally substituted with one or more halogen, hydroxy, (C₁-C₆)alkyl, (C₁-C₆)alkoxy, trifluoromethyl, cyano, or nitro, or
R⁶ is 2-pyridinyl, 3-pyridinyl, or 4-pyridinyl, each of which is optionally substituted with halogen, (C₁-C₆)alkyl, (C₁-C₆)alkoxy, trifluoromethyl, cyano, or nitro;
R³ is hydrogen; or
R¹ is hydrogen or (C₁-C₆)alkyl;
R² and R³ are identical and are each selected from (C₁-C₆)alkyl; or
R¹ and R³, together with the carbon to which they are attached, form a three- to six-membered carbocyclic ring; or
R¹ and R², together with the atoms to which R¹ and R² are attached, form a five- to seven-membered pyrrolidinyl-, piperidinyl-, or homopiperidinyl ring, or form a ring fragment selected from 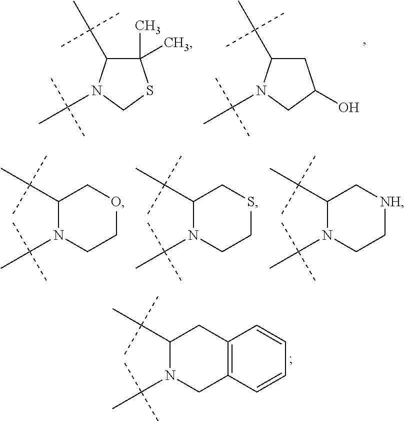
R³ is hydrogen;
R⁴ and R⁵ are independently selected from hydrogen, halogen, (C₁-C₆)alkyl, (C₁-C₆)alkoxy, hydroxy, trifluoromethyl, and cyano;
Q is R⁷—C(═O)—,
wherein
R⁷ is (C₁-C₆)alkyl optionally substituted with one or more hydroxy, (C₁-C₆)alkoxy, bis[(C₁-C₆)alkyl)]amino, or fluoro, or
R⁷ is R⁸(CH₂)ₙ,
wherein
n is 0 to 3,
R⁸ is phenyl optionally substituted with one or more halogen, hydroxy, (C₁-C₆)alkyl, (C₁-C₆)alkoxy, trifluoromethyl, cyano, or nitro, or
R⁸ is 2-pyridinyl, 3-pyridinyl, or 4-pyridinyl, each of which is optionally substituted with halogen, (C₁-C₆)alkyl, (C₁-C₆)alkoxy, trifluoromethyl, cyano, or nitro; or
R⁷ is R¹⁰C(R⁹)₂,
wherein
R⁹ is methyl or ethyl, or
C(R⁹)₂ is a 1,1-cyclopropyl, 1,1-cyclobutyl, 1,1-cyclopentyl, or 1,1-cyclohexyl ring,
R¹⁰ is phenyl optionally substituted with one or more halogen, hydroxy, (C₁-C₆)alkyl, (C₁-C₆)alkoxy, trifluoromethyl, cyano, or nitro, or
R¹⁰ is 2-pyridinyl, 3-pyridinyl, or 4-pyridinyl, each of which is optionally substituted with halogen, (C₁-C₆)alkyl, (C₁-C₆)alkoxy, trifluoromethyl, cyano, or nitro; or
R⁷ is a fragment group selected from 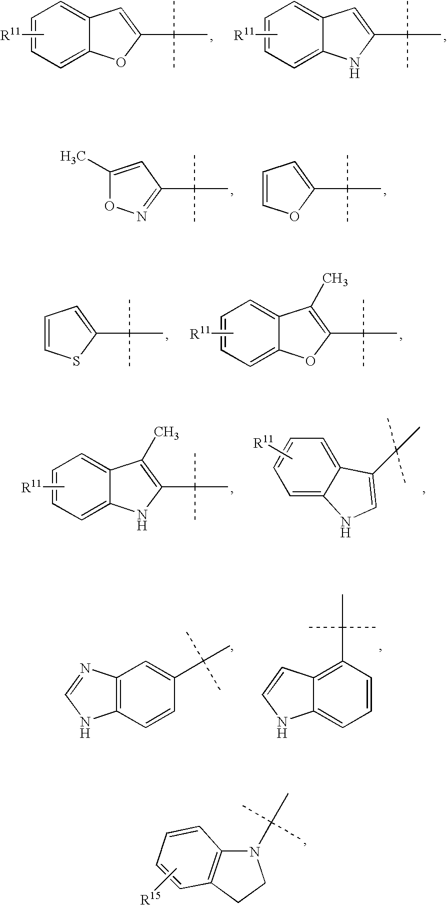
wherein
R¹¹ is one or more substituents selected from hydrogen, halogen, hydroxy, (C₁-C₆)alkyl, (C₁-C₆)alkoxy, trifluoromethyl, cyano, and nitro; or
Q is R¹³—N(R¹²)—C(═O)—,
wherein
R¹² is hydrogen or (C₁-C₆)alkyl,
R¹³ is (C₁-C₆)alkyl optionally substituted with one or more hydroxy, (C₁-C₆)alkoxy, bis[(C₁-C₆)alkyl)]amino, or fluoro; or
is R¹⁷(CH₂)ₚ,
wherein
p is 0 to 3,
R¹⁷ is phenyl optionally substituted with one or more halogen, hydroxy, (C₁-C₆)alkyl, (C₁-C₆)alkoxy, trifluoromethyl, trifluoromethoxy, cyano, or nitro, or
R¹⁷ is 2-pyridinyl, 3-pyridinyl, or 4-pyridinyl, each of which is optionally substituted with halogen, (C₁-C₆)alkyl, (C₁-C₆)alkoxy, trifluoromethyl, cyano, or nitro; or
R¹² and R¹³ and the nitrogen atom to which they are attached form a ring fragment, selected from 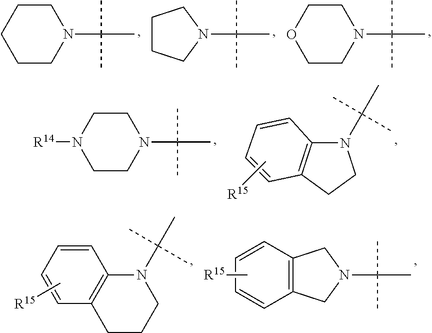
wherein
R¹⁴ is (C₁-C₆)alkyl; or
R¹⁴ is R¹⁶(CH₂)_{q};
wherein
q is 0 or 1,
R¹⁶ is phenyl optionally substituted with one or more halogen, hydroxy, (C₁-C₆)alkyl, (C₁-C₆)alkoxy, trifluoromethyl, trifluoromethoxy, cyano, or nitro, or
R¹⁶ is 2-pyridinyl, 3-pyridinyl, or 4-pyridinyl, each of which is optionally substituted with halogen, (C₁-C₆)alkyl, (C₁-C₆)alkoxy, trifluoromethyl, cyano, or nitro;
R¹⁵ is one or more substituents selected from halogen, hydroxy, (C₁-C₆)alkyl, (C₁-C₆)alkoxy, trifluoromethyl, cyano, and nitro;
or pharmaceutically acceptable salts and esters thereof,
with the proviso that Formula (I) is not N-{[4′-(2-methoxy-acetylamino)-1,1′-biphenyl-4-yl]-carbonyl}-L-phenylalanine.

2. The compound of claim 1, wherein
Y is C═O;
R¹ is hydrogen or (C₁-C₆)alkyl;
R² is (C₁-C₆)alkyl, hydroxy-(C₁-C₆)alkyl, (C₁₋)alkoxy-(C₁-C₆)alkyl, amino-C₁-C₆)alkyl, (C₁-C₆)alkylamino-(C₁-C₆)alkyl, or bis[(C₁-C₆)alkyl]amino-(C₁-C₆)alkyl;
R³ is hydrogen; or
R¹ is hydrogen or (C₁-C₆)alkyl;
R² is R⁶(CH₂M,
wherein
m is 0 to 3,
R⁶ is phenyl optionally substituted with one or more halogen, hydroxy, (C₁-C₆)alkyl, (C₁-C₆)alkoxy, trifluoromethyl, cyano, or nitro, or
R⁶ is 2-pyridinyl, 3-pyridinyl, or 4-pyridinyl, each of which is optionally substituted with halogen, (C₁-C₆)alkyl, (C₁-C₆)alkoxy, trifluoromethyl, cyano, or nitro;
R³ is hydrogen; or
R¹ is hydrogen or (C₁-C₆)alkyl;
R² and R³ are identical and are each selected from (C₁-C₆)alkyl; or
R² and R³, together with the carbon to which they are attached, form a three- to six-membered carbocyclic ring; or
R¹ and R², together with the atoms to which R¹ and R² are attached, form a five- to seven-membered pyrrolidinyl-, piperidinyl-, or homopiperidinyl ring, or form a ring fragment selected from 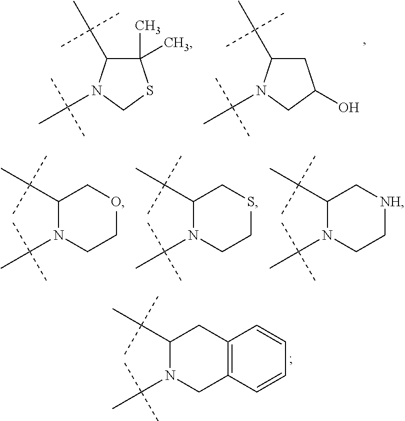
R³ is hydrogen;
R⁴ and R⁵ are independently selected from hydrogen, halogen, (C₁-C₆)alkyl, (C₁-C₆)alkoxy, hydroxy, trifluoromethyl, and cyano;
Q is R⁷—(═O)—,
wherein
R⁷ is (C₁-C₆)alkyl optionally substituted with one or more hydroxy, (C₁-C₆)alkoxy, bis[(C₁-C₆)alkyl)]amino, or fluoro, or
R⁷ is R⁸(CH₂)ₙ,
wherein
n is 0 to 3,
R⁸ is phenyl optionally substituted with one or more halogen, hydroxy, (C₁-C₆)alkyl, (C₁-C₆)alkoxy, trifluoromethyl, cyano, or nitro, or
R⁸ is 2-pyridinyl, 3-pyridinyl, or 4-pyridinyl, each of which is optionally substituted with halogen, (C₁-C₆)alkyl, (C₁₋)alkoxy, trifluoromethyl, cyano, or nitro; or
R⁷ is ROC(R)₂,
wherein
R⁹ is methyl or ethyl, or
C(R⁹)₂ is a 1,1-cyclopropyl, 1,1-cyclobutyl, 1,1-cyclopentyl, or 1,1-cyclohexyl ring,
R¹⁰ is phenyl optionally substituted with one or more halogen, hydroxy, (C₁-C₆)alkyl, (C₁-C₆)alkoxy, trifluoromethyl, cyano, or nitro, or
R¹⁰ is 2-pyridinyl, 3-pyridinyl, or 4-pyridinyl, each of which is optionally substituted with halogen, (C₁-C₆)alkyl, (C₁-C₆)alkoxy, trifluoromethyl, cyano, or nitro; or
R⁷ is a fragment group selected from 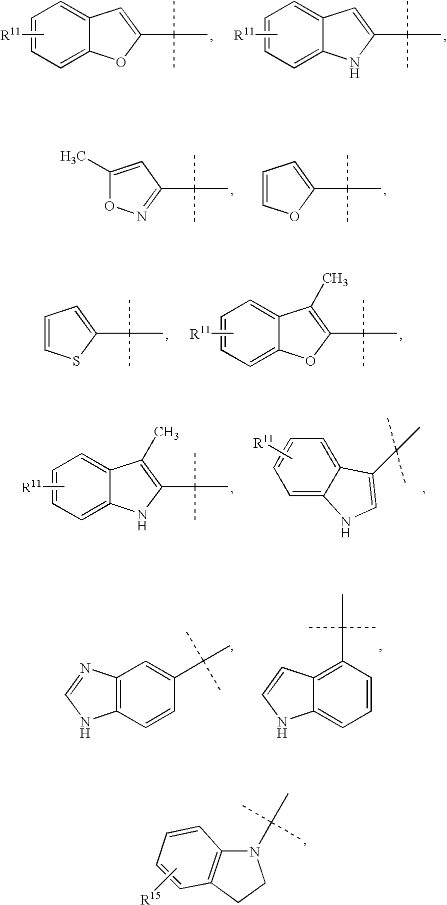
wherein
R¹¹ is one or more substituents selected from hydrogen, halogen, hydroxy, (C₁-C₆)alkyl, (C₁-C₆)alkoxy, trifluoromethyl, cyano, and nitro; or
Q is R¹³—N(R²)—C(═O)—,
wherein
R¹² is hydrogen or (C₁-C₆)alkyl,
R¹³ is (C₁-C₆)alkyl optionally substituted with one or more hydroxy, (C₁-C₆)alkoxy, bis[(C₁-C₆)alkyl)]amino, or fluoro; or
R¹³ is R¹⁷(CH₂)ₚ,
wherein
p is 0 to 3,
R¹⁷ is phenyl optionally substituted with one or more halogen, hydroxy, (C₁-C₆)alkyl, (C₁-C₆)alkoxy, trifluoromethyl, trifluoromethoxy, cyano, or nitro, or
R¹⁷ is 2-pyridinyl, 3-pyridinyl, or 4-pyridinyl, each of which is optionally substituted with halogen, (C₁-C₆)alkyl, (C₁-C₆)alkoxy, trifluoromethyl, cyano, or nitro; or
R¹² and R¹³ and the nitrogen atom to which they are attached form a ring fragment, selected from 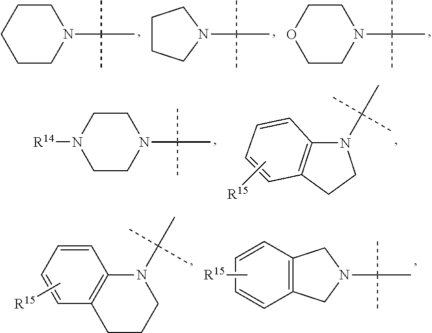
wherein
R¹⁴ is (C₁-C₆)alkyl; or
R¹⁴ is R¹⁶(CH₂)_{q},
wherein
q is 0 or 1,
R¹⁶ is phenyl optionally substituted with one or more halogen, hydroxy, (C₁-C₆)alkyl, (C₁-C₆)alkoxy, trifluoromethyl, trifluoromethoxy, cyano, or nitro, or
R¹⁶ is 2-pyridinyl, 3-pyridinyl, or 4-pyridinyl, each of which is optionally substituted with halogen, (C₁-C₆)alkyl, (C₁-C₆)alkoxy, trifluoromethyl, cyano, or nitro;
R¹⁵ is one or more substituents selected from halogen, hydroxy, (C₁-C₆)alkyl, (C₁-C₆)alkoxy, trifluoromethyl, cyano, and nitro;
or pharmaceutically acceptable salts and esters thereof.

3. The compound of claim 1, wherein
Y is S(═O)₂;
R¹ is hydrogen or (C₁-C₆)alkyl;
R² is (C₁-C₆)alkyl, hydroxy-(C₁-C₆)alkyl, (C₁-C₆)alkoxy-(C₁-C₆)alkyl, amino-(C₁-C₆)alkyl, (C₁-C₆)alkylamino-(C₁-C₆)alkyl, or bis[(C₁-C₆)alkyl]amino-(C₁-C₆)alkyl;
R³ is hydrogen; or
R¹ is hydrogen or (C₁-C₆)alkyl;
R² is R⁶(CH₂)ₘ,
wherein
m is 0 to 3,
R⁶ is phenyl optionally substituted with one or more halogen, hydroxy, (C₁-C₆)alkyl, (C₁-C₆)alkoxy, trifluoromethyl, cyano, or nitro, or
R⁶ is 2-pyridinyl, 3-pyridinyl, or 4-pyridinyl, each of which is optionally substituted with halogen, (C₁-C₆)alkyl, (C₁-C₆)alkoxy, trifluoromethyl, cyano, or nitro;
R³ is hydrogen; or
R¹ is hydrogen or (C₁-C₆)alkyl;
R² and R³ are identical and are each selected from (C₁-C₆)alkyl; or
R² and R³, together with the carbon to which they are attached, form a three- to six-membered carbocyclic ring; or
R¹ and R², together with the atoms to which R¹ and R² are attached, form a five to seven-membered pyrrolidinyl-, piperidinyl-, or homopiperidinyl ring, or form a ring fragment selected from 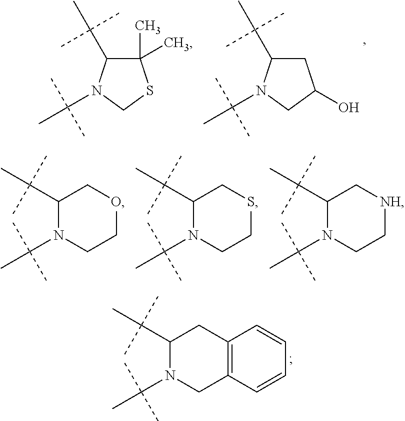
R³ is hydrogen;
R⁴ and R⁵ are independently selected from hydrogen, halogen, (C₁-C₆)alkyl, (C₁-C₆)alkoxy, hydroxy, trifluoromethyl, and cyano;
Q is R⁷—C(═O)—,
wherein
R⁷ is (C₁-C₆)alkyl optionally substituted with one or more hydroxy, (C₁-C₆)alkoxy, bis[(C₁-C₆)alkyl]amino, or fluoro, or
R⁷ is R⁸(CH₂)ₙ,
wherein
n is 0 to 3,
R⁸ is phenyl optionally substituted with one or more halogen, hydroxy, (C₁-C₆)alkyl, (C₁-C₆)alkoxy, trifluoromethyl, cyano, or nitro, or
R⁸ is 2-pyridinyl, 3-pyridinyl, or 4-pyridinyl, each of which is optionally substituted with halogen, (C₁-C₆)alkyl, (C₁-C₆)alkoxy, trifluoromethyl, cyano, or nitro; or
R⁷ is R¹⁰C(R)₂,
wherein
R⁹ is methyl or ethyl, or
C(R⁹)₂ is a 1,1-cyclopropyl, 1,1-cyclobutyl, 1,1-cyclopentyl, or 1,1-cyclohexyl ring,
R¹⁰ is phenyl optionally substituted with one or more halogen, hydroxy, (C₁-C₆)alkyl, (C₁-C₆)alkoxy, trifluoromethyl, cyano, or nitro, or
R¹⁰ is 2-pyridinyl, 3-pyridinyl, or 4-pyridinyl, each of which is optionally substituted with halogen, (C₁-C₆)alkyl, (C₁-C₆)alkoxy, trifluoromethyl, cyano, or nitro; or
R⁷ is a fragment group selected from 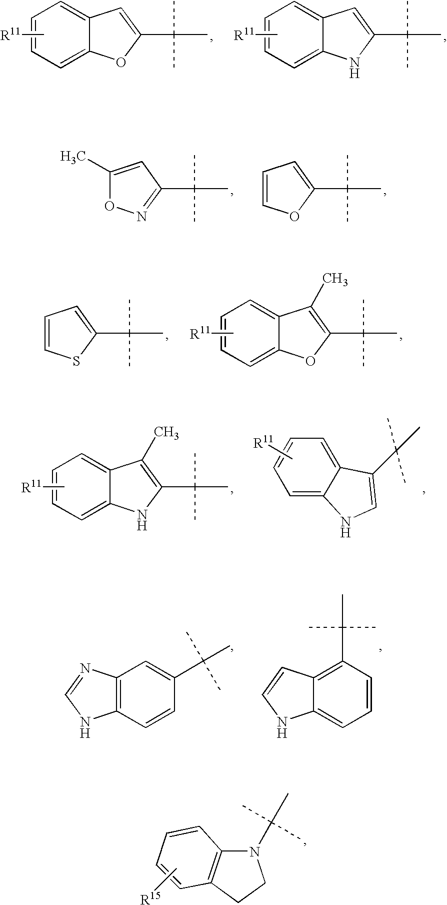
wherein
R¹¹ is one or more substituents selected from hydrogen, halogen, hydroxy, (C₁-C₆)alkyl, (C₁-C₆)alkoxy, trifluoromethyl, cyano, and nitro; or
Q is R¹³—N(R¹²)—C(═O)—,
wherein
R¹² is hydrogen or (C₁-C₆)alkyl,
R¹³ is (C₁-C₆)alkyl optionally substituted with one or more hydroxy, (C₁-C₆)alkoxy, bis[(C₁-C₆)alkyl)]amino, or fluoro; or
R¹³ is R¹⁷(CH₂)ₚ,
wherein
p is 0 to 3,
R¹⁷ is phenyl optionally substituted with one or more halogen, hydroxy, (C₁-C₆)alkyl, (C₁-C₆)alkoxy, trifluoromethyl, trifluoromethoxy, cyano, or nitro, or
R¹⁷ is 2-pyridinyl, 3-pyridinyl, or 4-pyridinyl, each of which is optionally substituted with halogen, (C₁-C₆)alkyl, (C₁-C₆)alkoxy, trifluoromethyl, cyano, or nitro; or
R¹² and R¹³ and the nitrogen atom to which they are attached form a ring fragment, selected from 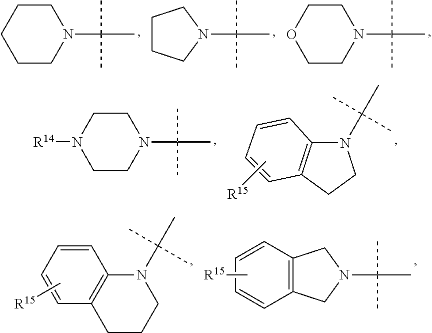
wherein
R¹⁴ is (C₁-C₆)alkyl; or
R¹⁴ is R¹⁶(CH₂)_{q},
wherein
q is 0 or 1,
R¹⁶ is phenyl optionally substituted with one or more halogen, hydroxy, (C₁-C₆)alkyl, (C₁-C₆)alkoxy, fluoromethyl, trifluoromethoxy, cyano, or nitro, or
R¹⁶ is 2-pyridinyl, 3-pyridinyl, or 4-pyridinyl, each of which is optionally substituted with halogen, (C₁-C₆)alkyl, (C₁-C₆)alkoxy, trifluoromethyl, cyano, or nitro;
R¹⁵ is one or more substituents selected from halogen, hydroxy, (C₁-C₆)alkyl, (C₁-C₆)alkoxy, trifluoromethyl, cyano, and nitro;
or pharmaceutically acceptable salts and esters thereof.

4. The compound of claim 1, wherein
Y is C═O or S(═O)₂;
R¹ is hydrogen or (C₁-C₆)alkyl;
R² is (C₁-C₆)alkyl, hydroxy-(C₁-C₆)alkyl, (C₁-C₆)alkoxy-(C₁-C₆)alkyl, amino-(C₁-C₆)alkyl, (C₁-C₆)alkylamino-(C₁-C₆)alkyl, or bis[(C₁-C₆)alkyl]amino-(C₁-C₆)alkyl;
R³ is hydrogen; or
R¹ is hydrogen or (C₁-C₆)alkyl;
R² is R⁶(CH₂)ₘ,
wherein
m is 0 to 3,
R⁶ is phenyl optionally substituted with one or more halogen, hydroxy, (C₁-C₆)alkyl, (C₁-C₆)alkoxy, trifluoromethyl, cyano, or nitro, or
R⁶ is 2-pyridinyl, 3-pyridinyl, or 4-pyridinyl, each of which is optionally substituted with halogen, (C₁-C₆)alkyl, (C₁-C₆)alkoxy, trifluoromethyl, cyano, or nitro;
R³ is hydrogen; or
R¹ is hydrogen or (C₁-C₆)alkyl;
R² and R³ are identical and are each selected from (C₁-C₆)alkyl; or
R² and R³, together with the carbon to which they are attached, form a three- to six-membered carbocyclic ring; or
R¹ and R², together with the atoms to which R¹ and R² are attached, form a five- to seven-membered pyrrolidinyl-, piperidinyl-, or homopiperidinyl ring, or form a ring fragment selected from 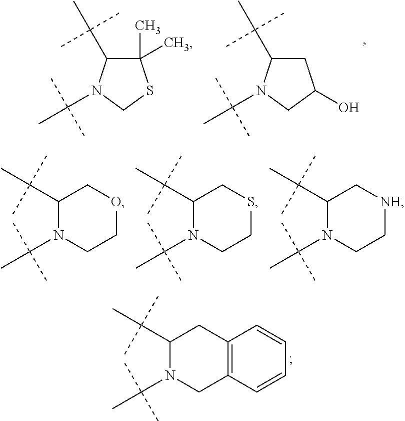
R³ is hydrogen;
R⁴ and R⁵ are independently selected from hydrogen, halogen, (C₁-C₆)alkyl, (C₁-C₆)alkoxy, hydroxy, trifluoromethyl, and cyano;
Q is R⁷—C(═O)—,
wherein
R⁷ is (C₁-C₆)alkyl optionally substituted with one or more hydroxy, (C₁-C₆)alkoxy, bis[(C₁-C₆)alkyl)]amino, or fluoro, or
R⁷ is R⁸(CH₂)ₙ,
wherein
n is 0 to 3,
R⁸ is phenyl optionally substituted with one or more halogen, hydroxy, (C₁-C₆)alkyl, (C₁-C₆)alkoxy, trifluoromethyl, cyano, or nitro, or
R⁸ is 2-pyridinyl, 3-pyridinyl, or 4-pyridinyl, each of which is optionally substituted with halogen, (C₁-C₆)alkyl, (C₁-C₆)alkoxy, trifluoromethyl, cyano, or nitro; or
R⁷ is R¹⁰C(R¹)₂,
wherein
R⁹ is methyl or ethyl, or
C(R)₂ is a 1,1-cyclopropyl, 1,1-cyclobutyl, 1,1-cyclopentyl, or 1,1-cyclohexyl ring,
R¹⁰ is phenyl optionally substituted with one or more halogen, hydroxy, (C₁-C₆)alkyl, (C₁-C₆)alkoxy, trifluoromethyl, cyano, or nitro, or
R¹⁰ is 2-pyridinyl, 3-pyridinyl, or 4-pyridinyl, each of which is optionally substituted with halogen, (C₁-C₆)alkyl, (C₁-C₆)alkoxy, trifluoromethyl, cyano, or nitro; or
R⁷ is a fragment group selected from 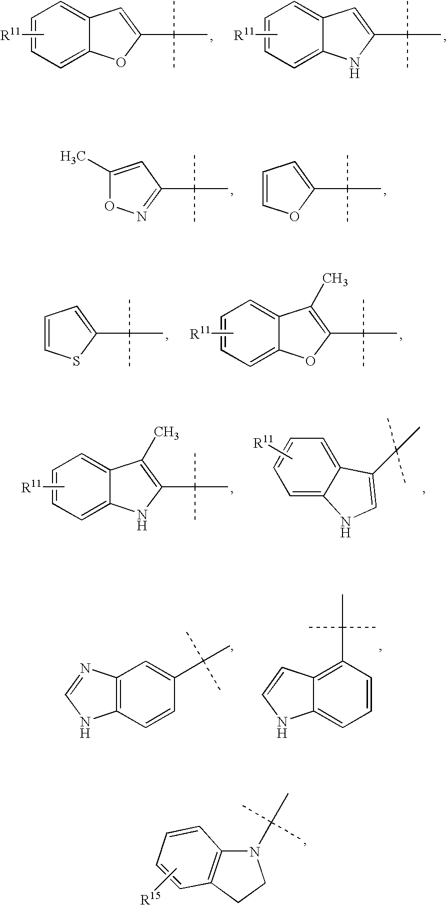
wherein
R¹¹ is one or more substituents selected from hydrogen, halogen, hydroxy, (C₁-C₆)alkyl, (C₁-C₆)alkoxy, trifluoromethyl, cyano, and nitro;
or pharmaceutically acceptable salts and esters thereof.

5. The compound of claim 1, wherein
Y is C═O or S(═O)₂;
R¹ is hydrogen or (C₁-C₆)alkyl;
R² is (C₁-C₆)alkyl, hydroxy-(C₁-C₆)alkyl, (C₁-C₆)alkoxy-C₁-C₆)alkyl, amino-(C₁-C₆)alkyl, (C₁-C₆)alkylamino-(C₁-C₆)alkyl, or bis[(C₁-C₆)alkyl]amino-(C₁-C₆)alkyl;
R³ is hydrogen; or
R¹ is hydrogen or (C₁-C₆)alkyl;
R² is R⁶(CH₂)ₘ,
wherein
m is 0 to 3,
R⁶ is phenyl optionally substituted with one or more halogen, hydroxy, (C₁-C₆)alkyl, (C₁-C₆)alkoxy, trifluoromethyl, cyano, or nitro, or
R⁶ is 2-pyridinyl, 3-pyridinyl, or 4-pyridinyl, each of which is optionally substituted with halogen, (C₁-C₆)alkyl, (C₁-C₆)alkoxy, trifluoromethyl, cyano, or nitro;
R³ is hydrogen; or
R¹ is hydrogen or (C₁-C₆)alkyl;
R² and R³ are identical and are each selected from (C₁-C₆)alkyl; or
R² and R³, together with the carbon to which they are attached, form a three- to six-membered carbocyclic ring; or
R¹ and R², together with the atoms to which R¹ and R² are attached, form a five- to seven-membered pyrrolidinyl-, piperidinyl-, or homopiperidinyl ring, or form a ring fragment selected from 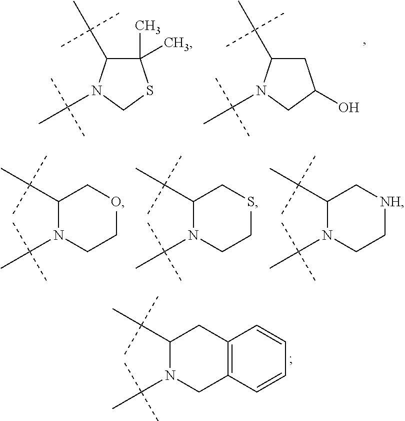
R³ is hydrogen;
R⁴ and R⁵ are independently selected from hydrogen, halogen, (C₁-C₆)alkyl, (C₁-C₆)alkoxy, hydroxy, trifluoromethyl, and cyano;
Q is R¹³—N(R¹²>C(═O)—,
wherein
R¹² is hydrogen or (C₁-C₆)alkyl,
R¹³ is (C₁-C₆)alkyl optionally substituted with one or more hydroxy, (C₁-C₆)alkoxy, bis[(C₁-C₆)alkyl)]amino, or fluoro; or
R¹³ is R¹⁷(CH₂)ₚ,
wherein
p is 0 to 3,
R¹⁷ is phenyl optionally substituted with one or more halogen, hydroxy, (C₁-C₆)alkyl, (C₁-C₆)alkoxy, trifluoromethyl, trifluoromethoxy, cyano, or nitro, or
R¹⁷ is 2-pyridinyl, 3-pyridinyl, or 4-pyridinyl, each of which is optionally substituted with halogen, (C₁-C₆)alkyl, (C₁-C₆)alkoxy, trifluoromethyl, cyano, or nitro; or
R¹² and R¹³ and the nitrogen atom to which they are attached form a ring fragment, selected from 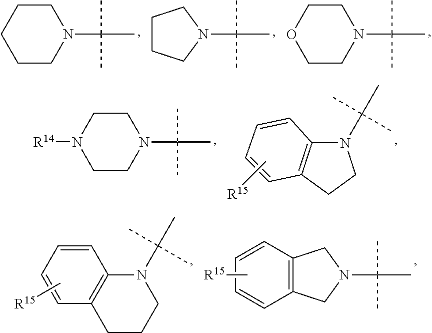
wherein
R¹⁴ is (C₁-C₆)alkyl; or
R¹⁴ is R¹⁶(CH₂)_{q},
wherein
q is 0 or 1,
R¹⁶ is phenyl optionally substituted with one or more halogen, hydroxy, (C₁-C₆)alkyl, (C₁-C₆)alkoxy, trifluoromethyl, trifluoromethoxy, cyano, or nitro, or
R¹⁶ is 2-pyridinyl, 3-pyridinyl, or 4-pyridinyl, each of which is optionally substituted with halogen, (C₁-C₆)alkyl, (C₁-C₆)alkoxy, trifluoromethyl, cyano, or nitro;
R¹⁵ is one or more substituents selected from halogen, hydroxy, (C₁-C₆)alkyl, (C₁-C₆)alkoxy, trifluoromethyl, cyano, and nitro;
or pharmaceutically acceptable salts and esters thereof.

6. The compound of claim 1, wherein
Y is C═O;
R¹ is hydrogen or (C₁-C₆)alkyl;
R² is (C₁-C₆)alkyl, hydroxy-(C₁-C₆)alkyl, (C₁-C₆)alkoxy-(C₁-C₆)alkyl, amino-(C₁-C₆)alkyl, (C₁-C₆)alkylamino-(C₁-C₆)alkyl, or bis[(C₁-C₆)alkyl]amino-C₁-C₆)alkyl;
R³ is hydrogen; or
R¹ is hydrogen or (C₁-C₆)alkyl;
R² is R⁶(CH₂)ₘ,
wherein
m is 0 to 3,
R⁶ is phenyl optionally substituted with one or more halogen, hydroxy, (C₁-C₆)alkyl, (C₁-C₆)alkoxy, trifluoromethyl, cyano, or nitro, or
R⁶ is 2-pyridinyl, 3-pyridinyl, or 4-pyridinyl, each of which is optionally substituted with halogen, (C₁-C₆)alkyl, (C₁-C₆)alkoxy, trifluoromethyl, cyano, or nitro;
R³ is hydrogen; or
R¹ is hydrogen or (C₁-C₆)alkyl;
R² and R³ are identical and are each selected from (C₁-C₆)alkyl; or
R² and R³, together with the carbon to which they are attached, form a three- to six-membered carbocyclic ring; or
R¹ and R², together with the atoms to which R¹ and R² are attached, form a five- to seven-membered pyrrolidinyl-, piperidinyl-, or homopiperidinyl ring, or form a ring fragment selected from 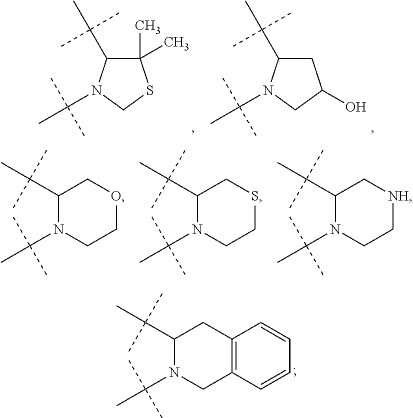
R³ is hydrogen;
R⁴ and R⁵ are independently selected from hydrogen, halogen, (C-Q)alkyl, (C₁-C₆)alkoxy, hydroxy, trifluoromethyl, and cyano;
Q is R⁷—C(═O)—,
wherein
R⁷ is (C₁-C₆)alkyl optionally substituted with one or more hydroxy, (C₁-C₆)alkoxy, bis[(C₁-C₆)alkyl)]amino, or fluoro, or
R⁷ is R(CH₇,
wherein
n is 0 to 3,
R⁸ is phenyl optionally substituted with one or more halogen, hydroxy, (C₁-C₆)alkyl, (C₁-C₆)alkoxy, trifluoromethyl, cyano, or nitro, or
R⁸ is 2-pyridinyl, 3-pyridinyl, or 4-pyridinyl, each of which is optionally substituted with halogen, (C₁-C₆)alkyl, (C₁-C₆)alkoxy, trifluoromethyl, cyano, or nitro; or
R⁷ is R¹⁰C(R⁹)₂,
wherein
R⁹ is methyl or ethyl, or
C(R⁹)₂ is a 1,1-cyclopropyl, 1,1-cyclobutyl, 1,1-cyclopentyl, or 1,1-cyclohexyl ring,
R¹⁰ is phenyl optionally substituted with one or more halogen, hydroxy, (C₁-C₆)alkyl, (C₁-C₆)alkoxy, trifluoromethyl, cyano, or nitro, or
R¹⁰ is 2-pyridinyl, 3-pyridinyl, or 4-pyridinyl, each of which is optionally substituted with halogen, (C₁-C₆)alkyl, (C₁-C₆)alkoxy, trifluoromethyl, cyano, or nitro; or
R⁷ is a fragment group selected from 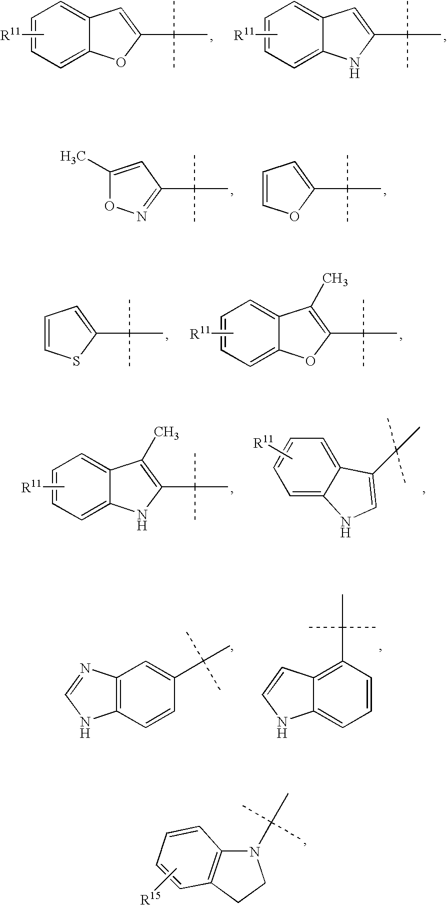
wherein
R¹¹ is one or more substituents selected from hydrogen, halogen, hydroxy, (C₁-C₆)alkyl, (C₁-C₆)alkoxy, trifluoromethyl, cyano, and nitro;
or pharmaceutically acceptable salts and esters thereof.

7. The compound of claim 1, wherein
Y is C═O;
R¹ is hydrogen or (C₁-C₆)alkyl;
R² is (C₁-C₆)alkyl, hydroxy-(C₁-C₆)alkyl, (C₁-C₆)alkoxy-(C₁-C₆)alkyl, amino-(C₁-C₆)alkyl, (C₁-C₆)alkylamino-(C₁-C₆)alkyl, or bis[(C₁-C₆)alkyl]amino-(C₁-C₆)alkyl;
R³ is hydrogen; or
R¹ is hydrogen or (C₁-C₆)alkyl;
R² is R⁶(CH₂)ₘ,
wherein
m is 0 to 3,
R⁶ is phenyl optionally substituted with one or more halogen, hydroxy, (C₁-C₆)alkyl, (C₁-C₆)alkoxy, trifluoromethyl, cyano, or nitro, or
R⁶ is 2-pyridinyl, 3-pyridinyl, or 4-pyridinyl, each of which is optionally substituted with halogen, (C₁-C₆)alkyl, (C₁-C₆)alkoxy, trifluoromethyl, cyano, or nitro;
R³ is hydrogen; or
R¹ is hydrogen or (C₁-C₆)alkyl;
R² and R³ are identical and are each selected from (C₁-C₆)alkyl; or
R² and R³, together with the carbon to which they are attached, form a three- to six-membered carbocyclic ring; or
R¹ and R², together with the atoms to which R¹ and R² are attached, form a five- to seven-membered pyrrolidinyl-, piperidinyl-, or homopiperidinyl ring, or form a ring fragment selected from 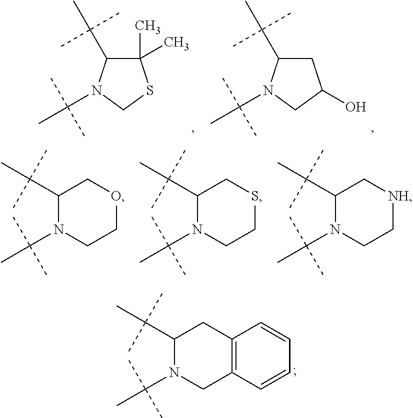
R³ is hydrogen;
R⁴ and R⁵ are independently selected from hydrogen, halogen, (C₁-C₆)alkyl, (C₁-C₆)alkoxy, hydroxy, trifluoromethyl, and cyano;
Q is R¹³—N(R¹²)—C(═O)—,
wherein
R¹² is hydrogen or (C₁-C₆)alkyl,
R¹³ is (C₁-C₆)alkyl optionally substituted with one or more hydroxy, (C₁-C₆)alkoxy, bis[(C₁-C₆)alkyl)]amino, or fluoro; or
R¹³ is R¹⁷(CH₂)ₚ,
wherein
p is 0 to 3,
R¹⁷ is phenyl optionally substituted with one or more halogen, hydroxy, (C₁-C₆)alkyl, (C₁-C₆)alkoxy, trifluoromethyl, trifluoromethoxy, cyano, or nitro, or
R¹⁷ is 2-pyridinyl, 3-pyridinyl, or 4-pyridinyl, each of which is optionally substituted with halogen, (C₁-C₆)alkyl, (C₁-C₆)alkoxy, trifluoromethyl, cyano, or nitro; or
R¹² and R¹³ and the nitrogen atom to which they are attached form a ring fragment, selected from 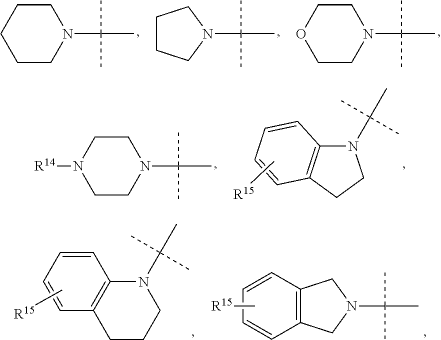
wherein
R¹⁴ is (C₁-C₆)alkyl; or
R¹⁴ is R¹⁶(CH₂)_{q},
wherein
q is 0 or 1,
R¹⁶ is phenyl optionally substituted with one or more halogen, hydroxy, (C₁-C₆)alkyl, (C₁-C₆)alkoxy, trifluoromethyl, trifluoromethoxy, cyano, or nitro, or
R¹⁶ is 2-pyridinyl, 3-pyridinyl, or 4-pyridinyl, each of which is optionally substituted with halogen, (C₁-C₆)alkyl, (C₁-C₆)alkoxy, trifluoromethyl, cyano, or nitro;
R¹⁵ is one or more substituents selected from halogen, hydroxy, (C₁-C₆)alkyl, (C₁-C₆)alkoxy, trifluoromethyl, cyano, and nitro;
or pharmaceutically acceptable salts and esters thereof.

8. The compound of claim 1, wherein
Y is S(═O)₂;
R¹ is hydrogen or (C₁-C₆)alkyl;
R² is (C₁-C₆)alkyl, hydroxy-(C₁-C₆)alkyl, (C₁-C₆)alkoxy-(C₁-C₆)alkyl, amino-(C₁-C₆)alkyl, (C₁-C₆)alkylamino-(C₁-C₆)alkyl, or bis[(C₁-C₆)alkyl]amino-(C₁-C₆)alkyl;
R³ is hydrogen; or
R¹ is hydrogen or (C₁-C₆)alkyl;
R² is R⁶(CH₂M,
wherein
m is 0 to 3,
R⁶ is phenyl optionally substituted with one or more halogen, hydroxy, (C₁-C₆)alkyl, (C₁-C₆)alkoxy, trifluoromethyl, cyano, or nitro, or
R⁶ is 2-pyridinyl, 3-pyridinyl, or 4-pyridinyl, each of which is optionally substituted with halogen, (C₁-C₆)alkyl, (C₁-C₆)alkoxy, trifluoromethyl, cyano, or nitro;
R³ is hydrogen; or
R¹ is hydrogen or (C₁-C₆)alkyl;
R² and R³ are identical and are each selected from (C₁-C₆)alkyl; or
R² and R³, together with the carbon to which they are attached, form a three- to six-membered carbocyclic ring; or
R¹ and R², together with the atoms to which R¹ and R² are attached, form a five- to seven-membered pyrrolidinyl-, piperidinyl-, or homopiperidinyl ring, or form a ring fragment selected from 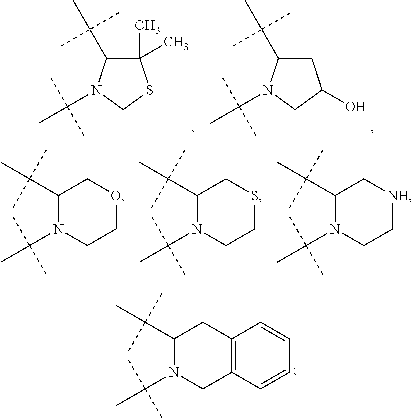
R³ is hydrogen;
R⁴ and R⁵ are independently selected from hydrogen, halogen, (C₁-C₆)alkyl, (C₁-C₆)alkoxy, hydroxy, trifluoromethyl, and cyano;
Q is R⁷—C(═O)—,
wherein
R⁷ is (C₁-C₆)alkyl optionally substituted with one or more hydroxy, (C₁-C₆)alkoxy, bis[(C₁-C₆)alkyl)]amino, or fluoro, or
R⁷ is R⁸(CH₂)ₙ,
wherein
n is 0 to 3,
R⁸ is phenyl optionally substituted with one or more halogen, hydroxy, (C₁-C₆)alkyl, (C₁-C₆)alkoxy, trifluoromethyl, cyano, or nitro, or
R⁸ is 2-pyridinyl, 3-pyridinyl, or 4-pyridinyl, each of which is optionally substituted with halogen, (C₁-C₆)alkyl, (C₁-C₆)alkoxy, trifluoromethyl, cyano, or nitro; or
is R¹⁰C(R⁹)₂,
wherein
R⁹ is methyl or ethyl, or
C(R⁹)₂ is a 1,1-cyclopropyl, 1,1-cyclobutyl, 1,1-cyclopentyl, or 1,1-cyclohexyl ring,
R¹⁰ is phenyl optionally substituted with one or more halogen, hydroxy, (C₁-C₆)alkyl, (C₁-C₆)alkoxy, trifluoromethyl, cyano, or nitro, or
R¹⁰ is 2-pyridinyl, 3-pyridinyl, or 4-pyridinyl, each of which is optionally substituted with halogen, (C₁-C₆)alkyl, (C₁-C₆)alkoxy, trifluoromethyl, cyano, or nitro; or
R⁷ is a fragment group selected from 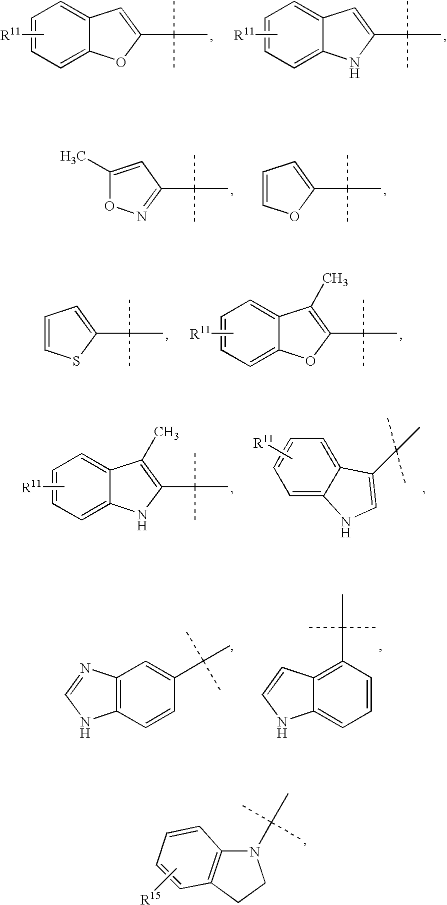
wherein
R¹¹ is one or more substituents selected from hydrogen, halogen, hydroxy, (C₁-C₆)alkyl, (C₁-C₆)alkoxy, trifluoromethyl, cyano, and nitro;
or pharmaceutically acceptable salts and esters thereof.

9. The compound of claim 1, wherein
Y is S(═O)₂;
R¹ is hydrogen or (C₁-C₆)alkyl;
is (C₁-C₆)alkyl, hydroxy-(C₁-C₆)alkyl, (C₁-C₆)alkoxy-(C₁-C₆)alkyl, amino-(C₁-C₆)alkyl, (C₁-C₆)alkylamino-C₁₋₆)alkyl, or bis[(C₁-C₆)alkyl]amino(C₁-C₆)alkyl;
R³ is hydrogen; or
R¹ is hydrogen or (C₁-C₆)alkyl;
R² is R⁶(CH₂)ₘ,
wherein
m is 0 to 3,
R⁶ is phenyl optionally substituted with one or more halogen, hydroxy, (C₁-C₆)alkyl, (C₁-C₆)alkoxy, trifluoromethyl, cyano, or nitro, or
R⁶ is 2-pyridinyl, 3-pyridinyl, or 4-pyridinyl, each of which is optionally substituted with halogen, (C₁-C₆)alkyl, (C₁-C₆)alkoxy, trifluoromethyl, cyano, or nitro;
R³ is hydrogen; or
R¹ is hydrogen or (C₁-C₆)alkyl;
R² and R³ are identical and are each selected from (C₁-C₆)alkyl; or
R² and R³, together with the carbon to which they are attached, form a three- to six-membered carbocyclic ring; or
R¹ and R², together with the atoms to which R¹ and R² are attached, form a five- to seven-membered pyrrolidinyl-, piperidinyl-, or homopiperidinyl ring, or form a ring fragment selected from 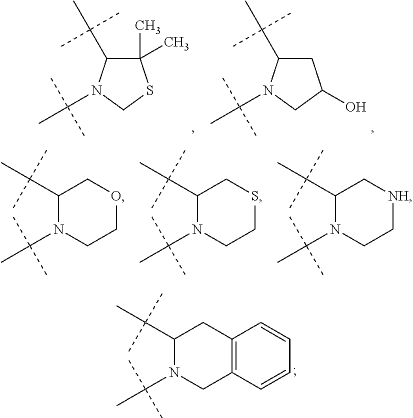
R³ is hydrogen;
R⁴ and R⁵ are independently selected from hydrogen, halogen, (C₁-C₆)alkyl, (C₁-C₆)alkoxy, hydroxy, trifluoromethyl, and cyano;
Q is R¹³—N(R¹²)—C(═O)—,
wherein
R¹² is hydrogen or (C₁-C₆)alkyl,
R¹³ is (C₁-C₆)alkyl optionally substituted with one or more hydroxy, (C₁-C₆)alkoxy, bis[(C₁-C₆)alkyl)]amino, or fluoro; or
R¹³ is R¹⁷(CH₂)ₚ,
wherein
p is 0 to 3,
R¹⁷ is phenyl optionally substituted with one or more halogen, hydroxy, (C₁-C₆)alkyl, (C₁-C₆)alkoxy, trifluoromethyl, trifluoromethoxy, cyano, or nitro, or
R¹⁷ is 2-pyridinyl, 3-pyridinyl, or 4-pyridinyl, each of which is optionally substituted with halogen, (C₁-C₆)alkyl, (C₁-C₆)alkoxy, trifluoromethyl, cyano, or nitro; or
R¹² and R¹³ and the nitrogen atom to which they are attached form a ring fragment, selected from 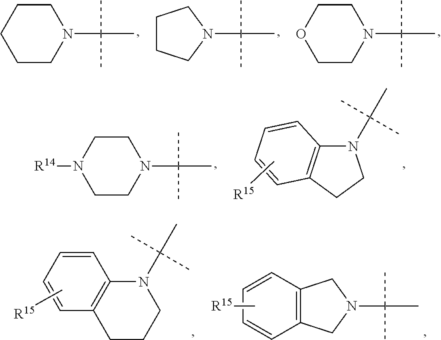
wherein
R¹⁴ is (C₁-C₆)alkyl; or
R¹⁴ is R¹⁶(CH₂)_{q},
wherein
q is 0 or 1,
R¹⁶ is phenyl optionally substituted with one or more halogen, hydroxy, (C₁-C₆)alkyl, (C₁-C₆)alkoxy, trifluoromethyl, trifluoromethoxy, cyano, or nitro, or
R¹⁶ is 2-pyridinyl, 3-pyridinyl, or 4-pyridinyl, each of which is optionally substituted with halogen, (C₁-C₆)alkyl, (C₁-C₆)alkoxy, trifluoromethyl, cyano, or nitro;
R¹⁵ is one or more substituents selected from halogen, hydroxy, (C₁-C₆)alkyl, (C₁-C₆)alkoxy, trifluoromethyl, cyano, and nitro;
or pharmaceutically acceptable salts and esters thereof.

10. The compound of claim 1, wherein
Y is C═O;
R¹ is hydrogen or (C₁-C₆)alkyl;
R² is (C₁-C₆)alkyl, hydroxy-(C₁-C₆)alkyl, (C₁-C₆)alkoxy-(C₁-C₆)alkyl, amino-(C₁-C₆)alkyl, (C₁-C₆)alkylamino-(C₁-C₆)alkyl, or bis[(C₁-C₆)alkyl]amino-(C₁-C₆)alkyl;
R³ is hydrogen; or
R¹ is hydrogen or (C₁-C₆)alkyl;
R² is R⁶(CH₂)ₘ,
wherein
m is 0 to 3,
R⁶ is phenyl optionally substituted with one or more halogen, hydroxy, (C₁-C₆)alkyl, (C₁-C₆)alkoxy, trifluoromethyl, cyano, or nitro,
R³ is hydrogen;
R⁴ and R⁵ are independently selected from hydrogen, halogen, (C₁-C₆)alkyl, (C₁-C₆)alkoxy, hydroxy, trifluoromethyl, and cyano;
Q is R⁷—C(═O)—,
wherein
R⁷ is (C₁-C₆)alkyl optionally substituted with one or more hydroxy, (C₁-C₆)alkoxy, bis[(C₁-C₆)alkyl)]amino, or fluoro, or
R⁷ is R⁸(CH₂)ₙ,
wherein
n is 0 to 3,
R⁸ is phenyl optionally substituted with one or more halogen, hydroxy, (C₁-C₆)alkyl, (C₁-C₆)alkoxy, trifluoromethyl, cyano, or nitro, or
R⁸ is 2-pyridinyl, 3-pyridinyl, or 4-pyridinyl, each of which is optionally substituted with halogen, (C₁-C₆)alkyl, (C₁-C₆)alkoxy, trifluoromethyl, cyano, or nitro; or
R⁷ is a fragment group selected from 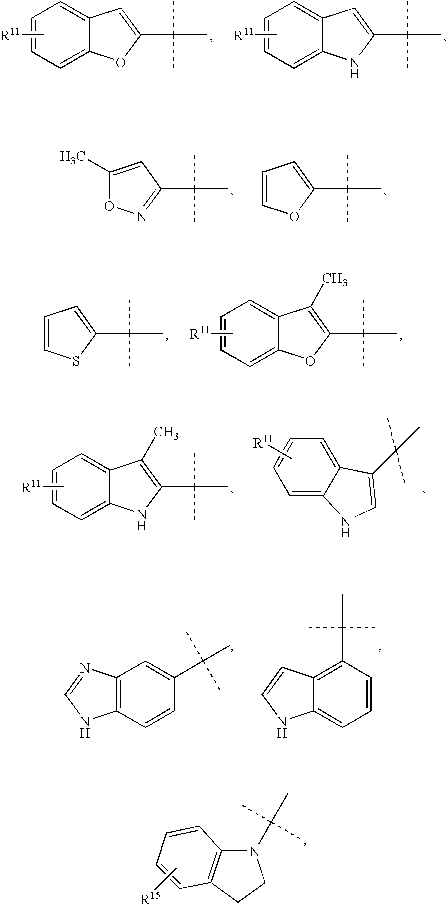
wherein
R¹¹ is one or more substituents selected from hydrogen, halogen, hydroxy, (C₁-C₆)alkyl, (C₁-C₆)alkoxy, trifluoromethyl, cyano, and nitro;
or pharmaceutically acceptable salts and esters thereof.

11. The compound of claim 1, wherein
Y is C═O;
R¹ is hydrogen or (C₁-C₆)alkyl;
R² and R³, together with the carbon to which they are attached, form a three- to six-membered carbocyclic ring; or
R¹ and R², together with the atoms to which R¹ and R² are attached, form a five- to seven-membered pyrrolidinyl-, piperidinyl-, or homopiperidinyl ring, or form a ring fragment selected from 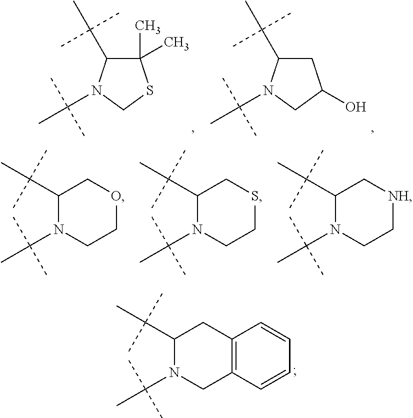
R³ is hydrogen;
R⁴ and R⁵ are independently selected from hydrogen, halogen, (C₁-C₆)alkyl, (C₁-C₆)alkoxy, hydroxy, trifluoromethyl, and cyano;
Q is R⁷—C(═O)—,
wherein
R⁷ is (C₁-C₆)alkyl optionally substituted with one or more hydroxy, (C₁-C₆)alkoxy, bis[(C₁-C₆)alkyl)]amino, or fluoro, or
R⁷ is R⁸(CH₂)ₙ,
wherein
n is 0 to 3,
R⁸ is phenyl optionally substituted with one or more halogen, hydroxy, (C₁-C₆)alkyl, (C₁-C₆)alkoxy, trifluoromethyl, cyano, or nitro, or
R⁸ is 2-pyridinyl, 3-pyridinyl, or 4-pyridinyl, each of which is optionally substituted with halogen, (C₁-C₆)alkyl, (C₁-C₆)alkoxy, trifluoromethyl, cyano, or nitro; or
R⁷ is a fragment group selected from 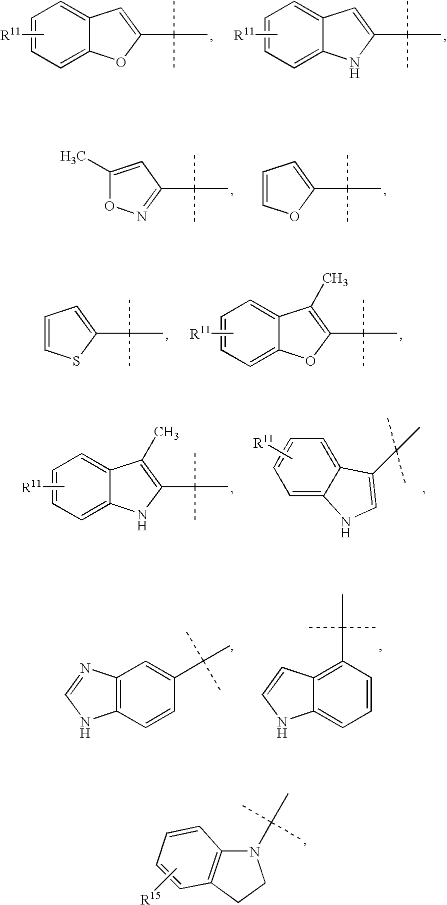
wherein
R¹¹ is one or more substituents selected from hydrogen, halogen, hydroxy, (C₁-C₆)alkyl, (C₁-C₆)alkoxy, trifluoromethyl, cyano, and nitro;
or pharmaceutically acceptable salts and esters thereof.

12. The compound of claim 1, wherein
Y is C═O;
R¹ is hydrogen or (C₁-C₆)alkyl;
R² is (C₁-C₆)alkyl, hydroxy-(C₁-C₆)alkyl, (C₁-C₆)alkoxy-(C₁-C₆)alkyl, amino(C₁-C₆)alkyl, (C₁-C₆)alkylamino-(C₁-C₆)alkyl, or bis[(C₁-C₆)alkyl]amino-(C₁-C₆)alkyl;
R³ is hydrogen; or
R¹ is hydrogen or (C₁-C₆)alkyl;
R² is R⁶(CH₂)ₘ,
wherein
m is 0 to 3,
R⁶ is phenyl optionally substituted with one or more halogen, hydroxy, (C₁-C₆)alkyl, (C₁-C₆)alkoxy, trifluoromethyl, cyano, or nitro, or
R⁶ is 2-pyridinyl, 3-pyridinyl, or 4-pyridinyl, each of which is optionally substituted with halogen, (C₁-C₆)alkyl, (C₁-C₆)alkoxy, trifluoromethyl, cyano, or nitro;
R³ is hydrogen;
R⁴ and R⁵ are independently selected from hydrogen, halogen, (C₁-C₆)alkyl, (C₁-C₆)alkoxy, hydroxy, trifluoromethyl, and cyano;
Q is R¹³—N(R¹²)—C(═O)—,
wherein
R¹² is hydrogen or (C₁-C₆)alkyl,
R¹³ is (C₁-C₆)alkyl optionally substituted with one or more hydroxy, (C₁-C₆)alkoxy, bis[(C₁-C₆)alkyl)]amino, or fluoro; or
R¹³ is R¹⁷(CH₂)ₚ,
wherein
p is 0 to 3,
R¹⁷ is phenyl optionally substituted with one or more halogen, hydroxy, (C₁-C₆)alkyl, (C₁-C₆)alkoxy, trifluoromethyl, trifluoromethoxy, cyano, or nitro, or
R¹⁷ is 2-pyridinyl, 3-pyridinyl, or 4-pyridinyl, each of which is optionally substituted with halogen, (C₁-C₆)alkyl, (C₁-C₆)alkoxy, trifluoromethyl, cyano, or nitro; or
R¹² and R¹³ and the nitrogen atom to which they are attached form a ring fragment, selected from 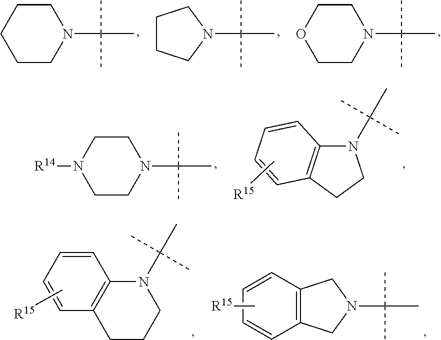
wherein
R¹⁴ is (C₁-C₆)alkyl; or
R¹⁴ is R¹⁶(CH₂)_{q},
wherein
q is 0 or 1,
R¹⁶ is phenyl optionally substituted with one or more halogen, hydroxy, (C₁-C₆)alkyl, (C₁-C₆)alkoxy, trifluoromethyl, trifluoromethoxy, cyano, or nitro, or
R¹⁶ is 2-pyridinyl, 3-pyridinyl, or 4-pyridinyl, each of which is optionally substituted with halogen, (C₁-C₆)alkyl, (C₁-C₆)alkoxy, trifluoromethyl, cyano, or nitro;
R¹⁵ is one or more substituents selected from halogen, hydroxy, (C₁-C₆)alkyl, (C₁-C₆)alkoxy, trifluoromethyl, cyano, and nitro;
or pharmaceutically acceptable salts and esters thereof.

13. The compound of claim 1, wherein
Y is C═O;
R¹ is hydrogen or (C₁-C₆)alkyl;
R² and R³ are identical and are each selected from (C₁-C₆)alkyl; or
R² and R³, together with the carbon to which they are attached, form a three- to six-membered carbocyclic ring; or
R¹ and R², together with the atoms to which R¹ and R² are attached, form a five- to seven-membered pyrrolidinyl-, piperidinyl-, or homopiperidinyl ring, or form a ring fragment selected from 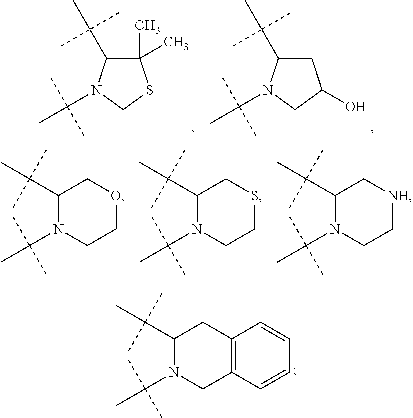
R³ is hydrogen;
R⁴ and R⁵ are independently selected from hydrogen, halogen, (C₁-C₆)alkyl, (C₁-C₆)alkoxy, hydroxy, trifluoromethyl, and cyano;
Q is R³—N(R¹²)—C(═O)—,
wherein
R¹² is hydrogen or (Cl_C₆)alkyl,
R¹³ is (C₁-C₆)alkyl optionally substituted with one or more hydroxy, (C₁-C₆)alkoxy, bis[(C₁-C₆)alkyl)]amino, or fluoro; or
R¹³ is R¹⁷(CH₂)ₚ,
wherein
p is 0 to 3,
R¹⁷ is phenyl optionally substituted with one or more halogen, hydroxy, (C₁-C₆)alkyl, (C₁-C₆)alkoxy, trifluoromethyl, trifluoromethoxy, cyano, or nitro, or
R¹⁷ is 2-pyridinyl, 3-pyridinyl, or 4-pyridinyl, each of which is optionally substituted with halogen, (C₁-C₆)alkyl, (C₁-C₆)alkoxy, trifluoromethyl, cyano, or nitro; or
R¹² and R¹³ and the nitrogen atom to which they are attached form a ring fragment, selected from 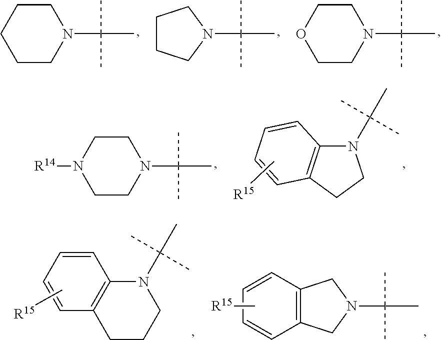
wherein
R¹⁴ is (C₁-C₆)alkyl; or
R¹⁴ is R¹⁶ (CH₂)_{q},
wherein
q is 0 or 1,
R¹⁶ is phenyl optionally substituted with one or more halogen, hydroxy, (C₁-C₆)alkyl, (C₁-C₆)alkoxy, trifluoromethyl, trifluoromethoxy, cyano, or nitro, or
R¹⁶ is 2-pyridinyl, 3-pyridinyl, or 4-pyridinyl, each of which is optionally substituted with halogen, (C₁-C₆)alkyl, (C₁-C₆)alkoxy, trifluoromethyl, cyano, or nitro;
R¹⁵ is one or more substituents selected from halogen, hydroxy, (C₁-C₆)alkyl, (C₁-C₆)alkoxy, trifluoromethyl, cyano, and nitro;
or pharmaceutically acceptable salts and esters thereof.

14. The compound of claim 1, wherein
Y is S(═O)₂;
R¹ is hydrogen or (C₁-C₆)alkyl;
R² is (C₁-C₆)alkyl, hydroxy-(C₁-C₆)alkyl, (C₁-C₆)alkoxy-(C₁-C₆)alkyl, amino-(C₁-C₆)alkyl, (C₁-C₆)alkylamino-(C₁-C₆)alkyl, or bis[(C₁-C₆)alkyl]amino-(C₁-C₆)alkyl;
R³ is hydrogen; or
R¹ is hydrogen or (C₁-C₆)alkyl;
R² is R⁶(CH₂)ₘ,
wherein
m is 0 to 3,
R⁶ is phenyl optionally substituted with one or more halogen, hydroxy, (C₁-C₆)alkyl, (C₁-C₆)alkoxy, trifluoromethyl, cyano, or nitro,
R³ is hydrogen;
R⁴ and R⁵ are independently selected from hydrogen, halogen, (C₁-C₆)alkyl, (C₁-C₆)alkoxy, hydroxy, trifluoromethyl, and cyano;
Q is R⁷—C(═O)—,
wherein
R⁷ is (C₁-C₆)alkyl optionally substituted with one or more hydroxy, (C₁-C₆)alkoxy, bis[(C₁-C₆)alkyl)]amino, or fluoro, or
R⁷ is R⁶(CH₂),
wherein
n is 0 to 3,
R⁸ is phenyl optionally substituted with one or more halogen, hydroxy, (C₁-C₆)alkyl, (C₁-C₆)alkoxy, trifluoromethyl, cyano, or nitro, or
R⁸ is 2-pyridinyl, 3-pyridinyl, or 4-pyridinyl, each of which is optionally substituted with halogen, (C₁-C₆)alkyl, (C₁-C₆)alkoxy, trifluoromethyl, cyano, or nitro; or
R⁷ is a fragment group selected from 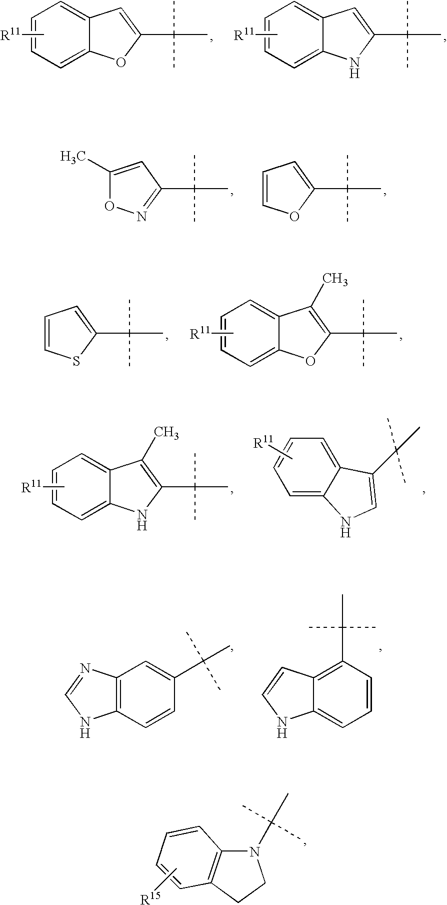
wherein
R¹¹ is one or more substituents selected from hydrogen, halogen, hydroxy, (C₁-C₆)alkyl, (C₁-C₆)alkoxy, trifluoromethyl, cyano, and nitro;
or pharmaceutically acceptable salts and esters thereof.

15. The compound of claim 1, wherein
Y is S(═O)₂;
R¹ is hydrogen or (C₁-C₆)alkyl;
R² and R³, together with the carbon to which they are attached, form a three- to six-membered carbocyclic ring; or
R¹ and R², together with the atoms to which R¹ and R² are attached, form a five- to seven-membered pyrrolidinyl-, piperidinyl-, or homopiperidinyl ring, or form a ring fragment selected from 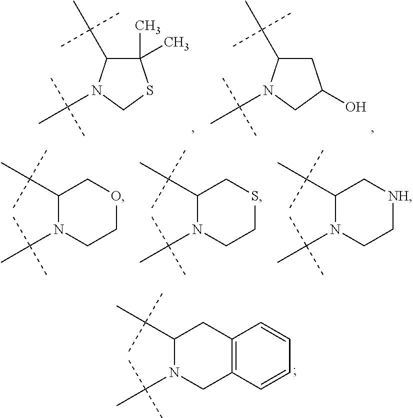
R³ is hydrogen;
R⁴ and R⁵ are independently selected from hydrogen, halogen, (C₁-C₆)alkyl, (C₁-C₆)alkoxy, hydroxy, trifluoromethyl, and cyano;
Q is R⁷—C(═O)—,
wherein
R⁷ is (C₁-C₆)alkyl optionally substituted with one or more hydroxy, (C₁-C₆)alkoxy, bis[(C₁-C₆)alkyl)]amino, or fluoro, or
R⁷ is R⁸(CH₂)ₙ,
wherein
n is 0 to 3,
R⁸ is phenyl optionally substituted with one or more halogen, hydroxy, (C₁-C₆)alkyl, (C₁-C₆)alkoxy, trifluoromethyl, cyano, or nitro, or
R⁸ is 2-pyridinyl, 3-pyridinyl, or 4-pyridinyl, each of which is optionally substituted with halogen, (C₁-C₆)alkyl, (C₁-C₆)alkoxy, trifluoromethyl, cyano, or nitro; or
R⁷ is a fragment group selected from 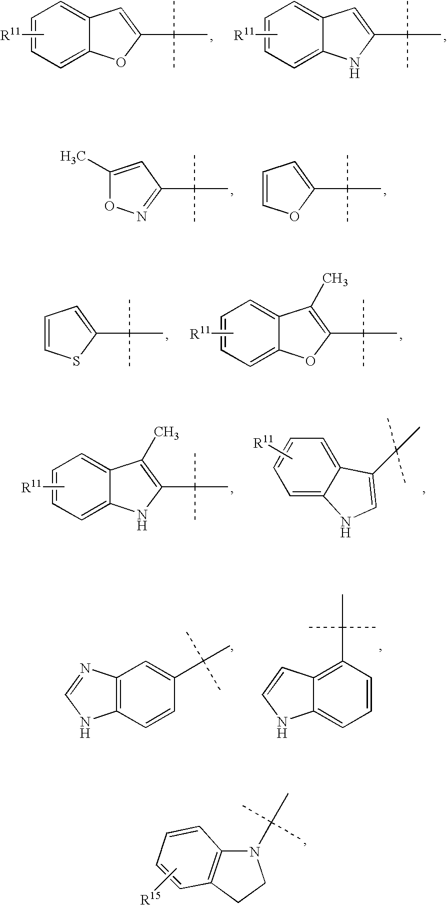
wherein
R¹¹ is one or more substituents selected from hydrogen, halogen, hydroxy, (C₁-C₆)alkyl, (C₁-C₆)alkoxy, trifluoromethyl, cyano, and nitro;
or pharmaceutically acceptable salts and esters thereof.

16. The compound of claim 1, wherein
Y is S(═O)₂;
R¹ is hydrogen or (C₁-C₆)alkyl;
R² is (C₁-C₆)alkyl, hydroxy-(C₁-C₆)alkyl, (C₁-C₆)alkoxy-(C₁-C₆)alkyl, amino-(C₁-C₆)alkyl, (C₁-C₆)alkylamino-(C₁-C₆)alkyl, or bis[(C₁-C₆)alkyl]amino-(C₁-C₆)alkyl;
R³ is hydrogen; or
R¹ is hydrogen or (C₁-C₆)alkyl;
R² is R⁶(CH₂)ₘ,
wherein
m is 0 to 3,
R⁶ is phenyl optionally substituted with one or more halogen, hydroxy, (C₁-C₆)alkyl, (C₁-C₆)alkoxy, trifluoromethyl, cyano, or nitro, or
R⁶ is 2-pyridinyl, 3-pyridinyl, or 4-pyridinyl, each of which is optionally substituted with halogen, (C₁-C₆)alkyl, (C₁-C₆)alkoxy, trifluoromethyl, cyano, or nitro;
R³ is hydrogen;
R⁴ and R⁵ am independently selected from hydrogen, halogen, (C₁₋₂)alkyl, (C₁-C₆)alkoxy, hydroxy, trifluoromethyl, and cyano;
Q is R¹³—N(R¹²)—C(O)—,
wherein
R¹² is hydrogen or (C₁-C₆)alkyl,
R¹³ is (C₁-C₆)alkyl optionally substituted with one or more hydroxy, (C₁-C₆)alkoxy, bis[(C₁-C₆)alkyl)]amino, or fluoro; or
R¹³ is R¹⁷(CH₂)ₚ,
wherein
p is 0 to 3,
R¹⁷ is phenyl optionally substituted with one or more halogen, hydroxy, (C₁-C₆)alkyl, (C₁-C₆)alkoxy, trifluoromethyl, trifluoromethoxy, cyano, or nitro, or
R¹⁷ is 2-pyridinyl, 3-pyridinyl, or 4-pyridinyl, each of which is optionally substituted with halogen, (C₁-C₆)alkyl, (C₁-C₆)alkoxy, trifluoromethyl, cyano, or nitro; or
R¹² and R¹³ and the nitrogen atom to which they are attached form a ring fragment, selected from 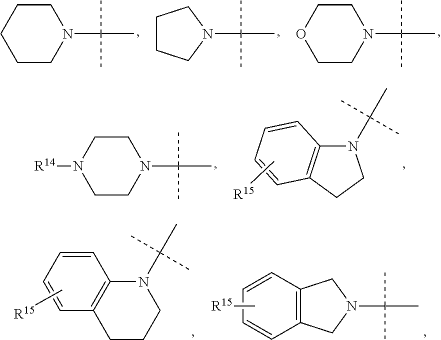
wherein
R¹⁴ is (C₁-C₆)alkyl; or
R¹⁴ is R¹⁶(CH₂)_{q},
wherein
q is 0 or 1,
R¹⁶ is phenyl optionally substituted with one or more halogen, hydroxy, (C₁-C₆)alkyl, (C₁-C₆)alkoxy, trifluoromethyl, trifluoromethoxy, cyano, or nitro, or
R¹⁶ is 2-pyridinyl, 3-pyridinyl, or 4-pyridinyl, each of which is optionally substituted with halogen, (C₁-C₆)alkyl, (C₁-C₆)alkoxy, trifluoromethyl, cyano, or nitro;
R¹⁵ is one or more substituents selected from halogen, hydroxy, (C₁-C₆)alkyl, (C₁-C₆)alkoxy, trifluoromethyl, cyano, and nitro;
or pharmaceutically acceptable salts and esters thereof.

17. The compound of claim 1, wherein
Y is S(═O)₂;
R¹ is hydrogen or (C₁-C₆)alkyl;
R² and R³ are identical and are each selected from (C₁-C₆)alkyl; or
R² and R³, together with the carbon to which they are attached, form a three- to six-membered carbocyclic ring; or
R¹ and R², together with the atoms to which R¹ and R² are attached, form a five- to seven-membered pyrrolidinyl-, piperidinyl-, or homopiperidinyl ring, or form a ring fragment selected from 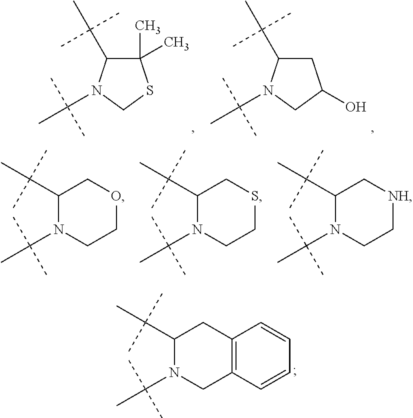
R³ is hydrogen;
R⁴ and R⁵ are independently selected from hydrogen, halogen, (C₁-C₆)alkyl, (C₁-C₆)alkoxy, hydroxy, trifluoromethyl, and cyano;
Q is R¹³—N(R¹²)—C(═O)—,
wherein
R¹² is hydrogen or (C₁-C₆)alkyl,
R¹³ is (C₁-C₆)alkyl optionally substituted with one or more hydroxy, (C₁-C₆)alkoxy, bis[(C₁-C₆)alkyl)]amino, or fluoro; or
R¹³ is R¹⁷(CH₂)ₚ,
wherein
p is 0 to 3,
R¹⁷ is phenyl optionally substituted with one or more halogen, hydroxy, (C₁-C₆)alkyl, (C₁-C₆)alkoxy, trifluoromethyl, trifluoromethoxy, cyano, or nitro, or
R¹⁷ is 2-pyridinyl, 3-pyridinyl, or 4-pyridinyl, each of which is optionally substituted with halogen, (C₁-C₆)alkyl, (C₁-C₆)alkoxy, trifluoromethyl, cyano, or nitro; or
R¹² and R¹³ and the nitrogen atom to which they are attached form a ring fragment, selected from 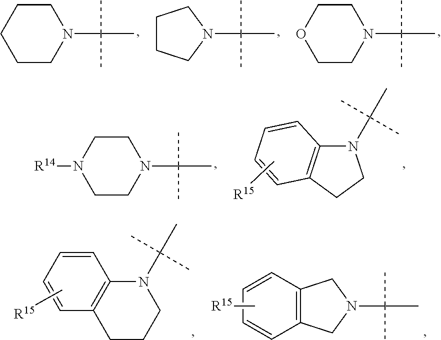
wherein
R¹⁴ is (C₁-C₆)alkyl; or
R¹⁴ is R¹⁶(CH₂)_{q},
wherein
q is 0 or 1,
R¹⁶ is phenyl optionally substituted with one or more halogen, hydroxy, (C₁-C₆)alkyl, (C₁-C₆)alkoxy, trifluoromethyl, trifluoromethoxy, cyano, or nitro, or
R¹⁶ is 2-pyridinyl, 3-pyridinyl, or 4-pyridinyl, each of which is optionally substituted with halogen, (C₁-C₆)alkyl, (C₁-C₆)alkoxy, trifluoromethyl, cyano, or nitro;
R¹⁵ is one or more substituents selected from halogen, hydroxy, (C₁-C₆)alkyl, (C₁-C₆)alkoxy, trifluoromethyl, cyano, and nitro;
or pharmaceutically acceptable salts and esters thereof,

18. The compound of claim 1 selected from the group consisting of
(2S)-1-{[4′-({[(2,3-dichlorophenyl)-amino]carbonyl}amino)-1,1′-biphenyl-4-yl]carbonyl}-2-piperidinecarboxylic acid,
(2S)-1-{[4′-({[(2,3-dimethylphenyl)amino]carbonyl}amino)-1,1′-biphenyl-4-yl]carbonyl}-2-piperidinecarboxylic acid,
(2S)-1-{[4′-({[(2,4-dichlorophenyl)-amino]carbonyl}amino)-1,1′-biphenyl-4-yl]carbonyl}-2-piperidinecarboxylic acid,
(2S)-1-{[4′-({[(2,4-difluorophenyl)-amino]carbonyl}amino)-1,1′-biphenyl-4-yl]carbonyl}-2-piperidinecarboxylic acid,
(2S)-1-{[4′-({[(4-butylphenyl)-amino]carbonyl}amino)-1,1′-biphenyl-4-yl]carbonyl}-2-piperidinecarboxylic acid,
(2S)-1-{[4′-({[(2,4-dimethylphenyl)-amino]carbonyl}amino)-1,1′-biphenyl-4-yl]carbonyl}-2-piperidinecarboxylic acid,
(2S)-1-{[4′-({[(2,5-dichlorophenyl)-amino]carbonyl}amino)-1,1′-biphenyl-4-yl]carbonyl}-2-piperidinecarboxylic acid,
(2S)-1-([4′-(([(2,6-dichlorophenyl)-amino]carbonyl)amino)-1,1′-biphenyl-4-yl]carbonyl)-2-piperidinecarboxylic acid,
(2S)-1-{[4′-({[(2,6-dimethylphenyl)-amino]carbonyl}amino)-1,1′-biphenyl-4-yl]carbonyl}-2-piperidinecarboxylic acid,
(2S)1-{[4′-({[(2-trifluoromethoxyphenyl)amino]carbonyl}amino)-1,1′-biphenyl-4-yl]carbonyl}-2-piperidinecarboxylic acid,
(2S)-1-{[4′-({[(3,4-dichlorophenyl)-amino]carbonyl}amino)-1,1′-biphenyl-4-yl]carbonyl}-2-piperidinecarboxylic acid,
(2S)-1-{[4′-({[(3,4-dimethylphenyl)-amino]carbonyl}amino)-1,1′-biphenyl-4-yl]carbonyl}-2-piperidinecarboxylic acid,
(2S)-1-{[4′-({[(3,5-dichlorophenyl)-amino]carbonyl}amino)-1,1′-biphenyl-4-yl]carbonyl}-2-piperidinecarboxylic acid,
(2S)-1-{[4′-({[(3,5-dimethylphenyl)-amino]carbonyl}amino)-1,1′-biphenyl-4-yl]carbonyl}-2-piperidinecarboxylic acid,
(2S)-1-{[4′-({[(2-methoxyphenyl)-amino]carbonyl}amino)-1,1′-biphenyl-4-yl]carbonyl}-2-piperidinecarboxylic acid,
(2S)-1-{[4′-({[(4-trifluoromethoxyphenyl)-amino]carbonyl}amino)-1,1′-biphenyl-4-yl]carbonyl}-2-piperidinecarboxylic acid,
1-({[4′-([(2,4-dichlorophenyl)-amino]carbonyl]-amino)-1,1′-biphenyl-4-yl]carbonyl}amino)cyclopropanecarboxylic acid,
1-({[4′-({[(2,41-fluorophenyl)-amino]carbonyl}amino)-1,1′-biphenyl-4-yl]carbonyl}amino)cyclopropanecarboxylic acid,
1-({[4′-({[(3,44-dimethylphenyl)amino]carbonyl}amino)-1,1′-biphenyl-4-yl]carbonyl}amino)cyclopropanecarboxylic acid, and
1-({[4′-({[(2,4-dimethylphenyl)-amino]carbonyl}amino)-1,1′-biphenyl-4-yl]carbonyl}amino)cyclopropanecarboxylic acid.

19. The compound of claim 1 selected from the group consisting of
1-({[4′-({[(2-chlorophenyl)-amino]carbonyl}amino)-1,1′-biphenyl-4-yl]carbonyl}-amino) cyclopropanecarboxylic acid,
1-({[4′-({[(2-ethoxyphenyl)-amino]carbonyl}-amino)-1,1′-biphenyl-4-yl]carbonyl}amino) cyclopropanecarboxylic acid,
1-({[4′-({[(3,4-dichlorophenyl)-amino]carbonyl}amino)-1,1′-biphenyl-4-yl]carbonyl}amino) cyclopropanecarboxylic acid,
1-({4′-({[(3,4-dimethylphenyl)-amino]carbonyl}amino)-1,1′-biphenyl-4-yl]carbonyl}amino) cyclopropanecarboxylic acid,
1-({[4′-({[(4-butylphenyl)-amino]carbonyl}amino)-1,1′-biphenyl-4-yl]carbonyl}amino)cyclopropanecarboxylic acid,
1-({[4′-({[(4-ethylphenyl)-amino]carbonyl}amino)-1,1′-biphenyl-4-yl]carbonyl}amino) cyclopropanecarboxylic acid,
1-({[4′ ({[(4-fluoro-3-methylphenyl)amino]carbonyl}amino)-1,1′-biphenyl-4-yl]carbonyl}amino)cyclopropanecarboxylic acid,
1-({[4′-(pentanoylamino)biphenyl-4-yl]carbonyl}amino)cyclopropanecarboxylic acid,
1-[({4′-[((4-chlorophenyl)-acetyl)amino]-1,1′-biphenyl-4-yl}carbonyl)amino]cyclopropanecarboxylic acid,
1-[({4′-[(4-butylbenzoyl)amino]-1,1′-biphenyl-4-yl}carbonyl)amino]cyclopropanecarboxylic acid,
1-[({4′-[(4-chlorobenzoyl)amino]-1,1′-biphenyl-4-yl}carbonyl)amino]cyclopropanecarboxylic acid,
N-{[4′-({[(4-ethylphenyl)-amino]carbonyl}amino)biphenyl-4-yl]carbonyl}-L-valine,
1-[(4′-({[(2,4-difluorophenyl)-acetyl]amino}-1,1′-biphenyl-4-yl)carbonyl]-L-proline,
N-{[4′ {[(2,3-dichlorophenyl)-amino]carbonyl}amino)biphenyl-4-yl]carbonyl}-L-valine,
1-[(4′-({[(3,5-difluorophenyl)-acetyl]amino}-1,1′-biphenyl-4-yl)carbonyl]-L-proline,
1-[(4′-({[(5-methylisoxazol-3-yl)carbonyl]amino}biphenyl-4-yl)carbonyl]-L-proline,
1-{[4′-({[(2,3-dichlorophenyl)-amino]carbonyl}amino)1,1′-biphenyl-4-yl]carbonyl}-L-proline,
1-{[4′-({[(2,3-dichlorophenyl)-amino]carbonyl}amino)-1,1′-biphenyl-4-yl]carbonyl}-D-proline,
1-{[4′-({[(2,3-dimethylphenyl)-amino]carbonyl}amino)-1,1′-biphenyl-4-yl]carbonyl}-D-proline,
1-{[4′-({[(2,4-dichlorophenyl)-amino]carbonyl}amino)-1,1′-biphenyl-4-yl]carbonyl}-D-proline, and
1-{[4′-({[(2,4-difluorolphenyl)-amino]carbonyl}amino)-1,1′-biphenyl-4-yl]carbonyl}-D-proline.

20. The compound of claim 1 selected from the group consisting of
1-{[4′-({[(2,4-difluorolphenyl)-amino]carbonyl}amino)-1,1′-biphenyl-4-yl]carbonyl}-L-proline,
1-{[4′-({[(2,4-dimethylphenyl)-amino]carbonyl}amino)-1,1′-biphenyl-4-yl]carbonyl}-D-proline,
1-{[4′-({[(2,4-dimethylphenyl)-amino]carbonyl}amino)-11,1′-biphenyl-4-yl]carbonyl}-L-proline,
1-{[4′-({[(2,5-dichlorophenyl)-amino]carbonyl}amino)-1,1′-biphenyl-4-yl]carbonyl}-D-proline,
1-{[4′-({[(2,5-difluorophenyl)-amino]carbonyl}amino)-1,1′-biphenyl-4-yl]carbonyl}-D-proline,
1-{[4′-({[(2,6-dichlorophenyl)-amino]carbonyl}amino)-1,1′-biphenyl-4-yl]carbonyl}-D-proline,
1-{[4′-({[(2,6-dimethylphenyl)-amino]carbonyl}amino)-1,1′-biphenyl-4-yl]carbonyl}-D-proline,
1-{[4′-({[(3,4-dichlorophenyl)-amino]carbonyl}amino)-1,1′-biphenyl-4-yl]carbonyl}-D-proline,
1-{[4′-({[(3,4-dimethylphenyl)-amino]carbonyl}amino)-1,1′-biphenyl-4-yl]carbonyl}-D-proline,
1-{[4′-({[(3,5-dichlorophenyl)-amino]carbonyl}amino)-1,1′-biphenyl-4-yl]carbonyl}-D-proline,
1-{[4′-({[(4-butylphenyl)-amino]carbonyl} amino)-1,1′-biphenyl-4-yl]carbonyl}-D-proline,
1-{[4′-({[(2-methoxyphenyl)-amino]carbonyl}amino)-1,1′-biphenyl-4-yl]carbonyl}-D-proline,
1-{[4′-(pentanoylamino)carbonyl}amino)-1,1′-biphenyl-4-yl]carbonyl}-L-proline,
2-methyl-N-({4′-[({[4-(trifluoromethyl)phenyl]amino}carbonyl)amino]biphenyl-4-yl}carbonyl)alanine,
2-methyl-N-[(4′-{[(pyridin-3-ylamino)carbonyl]amino}biphenyl-4-yl)carbonyl]alanine,
N-({4′-(pentanoylamino)-1,1′-biphenyl-4-yl}carbonyl)-D-valine,
N-({4′-[((2,4-difluorophenyl)acetyl)amino]-1,1′-biphenyl-4-yl}carbonyl)-L-valine,
N-({4′-[(2-fluorobenzoyl)amino]-1,1′-biphenyl-4-yl}carbonyl)-N-methyl-L-valine,
N-({4′-[(2-fluorobenzoyl)amino]biphenyl-4-yl}carbonyl)-D-valine, and
N-({4′-[(2-fluorobenzoyl)amino]biphenyl-4-yl}carbonyl)-N,2-dimethylalanine.

21. The compound of claim 1 selected from the group consisting of
N-({4′-[(3,4-dichlorobenzoyl)amino]-1,1′-biphenyl-4-yl}carbonyl)-D-valine,
N-({4-[(3,4-dichlorobenzoyl)amino]-1,1′-biphenyl-4-yl}carbonyl)-N-methyl-L-alanine,
N-({-4′-[(3,4-dichlorobenzoyl)amino]-1,1′-biphenyl-4-yl}carbonyl)-N-methyl-L-valine,
N-({4′-[(3,4-difluorobenzoyl)amino]-1,1′-biphenyl-4-yl}carbonyl)-D-valine,
N-({4′-[(3,4-difluorobenzoyl)amino]-1,1′-biphenyl-4-yl}carbonyl)-N-methyl-L-alanine,
N-({4′-[(3,4-dimethylbenzoyl)amino]-1,1′-biphenyl-4-yl}carbonyl)-D-valine,
N-({4′-[(3,4-dimethylbenzoyl)amino]-1,1′-biphenyl-4-yl}carbonyl)-L-valine,
N-({4′-[(3,4-dimethylbenzoyl)amino]-1,1′-biphenyl-4-yl]carbonyl}-N-methyl-L-alanine,
N-({4′-[(3,4-dimethylbenzoyl)amino]-1,1′-biphenyl-4-yl}carbonyl)-N-methyl-L-valine,
N-({4′-[(3,4-dimethylbenzoyl)amino]biphenyl-4-yl}carbonyl)-N,2-dimethylalanine,
N-({4′-[(3,5-difluorobenzoyl)amino]-1,1′-biphenyl-4-yl}carbonyl)-D-vale,
N-({4′-[(3,5-difluorobenzoyl)amino]-1,1′-biphenyl-4-yl}carbonyl)-N-methyl-L-alanine,
N-({4′-[(3,5-difluorobenzoyl)amino]biphenyl-4-yl}carbonyl)-N,2-dimethylalanine,
N-({4′-[(3,5-dimethoxybenzoyl)amino]-1,1′-biphenyl-4-yl}carbonyl)-N-methyl-L-valine,
N-({4′-[(3,5-dimethoxybenzoyl)amino]biphenyl-4-yl]carbonyl}-N,2-dimethylalanine,
N-({4′-[(3-fluoro-4-methylbenzoyl)amino]biphenyl-4-yl]carbonyl}-N,2-dimethylalanine,
N-({4′-[(3-methylbutanoyl)amino]-1,1′-biphenyl-4-yl}carbonyl)-N-methyl-L-valine,
N-({4′-[(4-butylbenzoyl)amino]-1,1′-biphenyl-4-yl}carbonyl)-D-valine,
N-({4′-[(4-butylbenzoyl)amino]-1,1′-biphenyl-4-yl}carbonyl)-L-valine, and
N-({4′-[(4-butylbenzoyl)amino]-1,1′-biphenyl-4-yl}carbonyl)-N-methyl-L-alanine.

22. The compound of claim 1 selected from the group consisting of
N-({4′-[(4-butylbenzoyl)amino]biphenyl-4-yl]carbonyl}-N,2-dimethylalanine,
N-({4′-[(4-chlorobenzoyl)amino]-1,1′-biphenyl-4-yl}carbonyl)-D-valine,
N-({4′-[(4-chlorobenzoyl)amino]-1,1′-biphenyl-4-yl}carbonyl)-N-methyl-L-alanine,
N-({4′-[(4-chlorobenzoyl)amino]-1,1′-biphenyl-4-yl}carbonyl)-N-methyl-L-valine,
N-({4′-[(4-chlorobenzoyl)amino]biphenyl-4-yl}carbonyl)-N,2-dimethylalanine,
N-({4′-[(4-ethylbenzoyl)amino]-1,1′-biphenyl-4-yl}carbonyl)-N-methyl-L-alanine,
N-({4′-[(4-ethylbenzoyl)amino]-1,1′-biphenyl-4-yl}carbonyl)-N-methyl-L-valine,
N-({4′-[(4-ethylbenzoyl)amino]biphenyl-4-yl}carbonyl)-N,2-dimethylalanine,
N-({4′-[(4-ethyl]benzoyl)amino]-1,1′-biphenyl-4-yl}carbonyl)-D-valine,
N-({4′-[(4-fluoro-3-methylbenzoyl)amino]-1,1′-biphenyl-4-yl}carbonyl)-D-valine,
N-({4′-[(4-fluoro-3-methylbenzoyl)amino]-1,1′-biphenyl-4-yl}carbonyl)-L-valine,
N-({4′-[(4-fluoro-3-methylbenzoyl)amino]-1,1′-biphenyl-4-yl}carbonyl)-N-methyl-L-alanine,
N-({4′-[(4-fluoro-3-methylbenzoyl)amino]-1,1′-biphenyl-4-yl}carbonyl)-N-methyl-L-valine,
N-({4′-[(4-fluoro-3-methylbenzoyl)amino]biphenyl-4-yl}carbonyl)-N,2-dimethylalanine,
N-({4′-[(4-fluorobenzoyl)amino]-1,1′-biphenyl-4-yl}carbonyl)-D-valine,
N-{4′-[(4-fluorobenzoyl)arm-o]-1,1′-biphenyl-4-yl}carbonyl)-N-methyl-L-alanine,
N-({4′-[(4-fluorobenzoyl)amino]-1,1′-biphenyl-4-yl}carbonyl)-N-methyl-L-valine,
N-({4′-[(4-fluorobenzoyl)amino]biphenyl-4-yl}carbonyl)-N,2-dimethylalanine,
N-({4′-[(4-methylpentanoyl)amino]-1,1′-biphenyl-4-yl}carbonyl)-L-valine, and
N-({4′-[(anilinocarbonyl)amino]biphenyl-4-yl}carbonyl)-2-methylalanine.

23. The compound of claim 1 selected from the group consisting of
N,2-dimethyl-N-({4′-[(2-methylbenzoyl)amino]biphenyl-4-yl}carbonyl)alanine,
N,2-dimethyl-N-({4′-[(3-methylbutanoyl)amino]biphenyl-4-yl}carbonyl)alanine,
N,2-dimethyl-N-({4′-[4-methylbenzoyl)amino]biphenyl-yl}carbonyl)alanine,
N,2-dimethyl-N-({4′-[(4-methylpentanoyl)amino]biphenyl-4-yl}carbonyl)alanine,
N,2-dimethyl-N-{[4′-(pentanoylamino)biphenyl-4-yl]carbonyl}alanine,
N-[(4′-({[(1-benzofuran-2-yl)carbonyl]amino}-1,1′-biphenyl-4-yl)carbonyl]-L-valine,
N-[(4′-({[(2,4-difluorophenyl)-acetyl]amino}-1,1′-biphenyl-4-yl)carbonyl]-D-valine,
N-[(4′-({[(3,4-dimethoxyphenyl)-acetyl]amino}-1,1′-biphenyl-4-yl)carbonyl]-D-valine,
N-[(4′-({[(3,4-dimethoxyphenyl)-acetyl]amino}biphenyl-4-yl) carbonyl]-N,2-dimethylalanine,
N-[(4′-{[(3,5-difluorophenyl)-acetyl]amino}-1,1′-biphenyl-4-yl)carbonyl]-D-valine,
N-[(4′-{[(3,5-difluorophenyl)-acetyl]amino}-1,1′-biphenyl-4-yl)carbonyl]-L-valine,
N-[(4′-{[(3,5-difluorophenyl)-acetyl]amino}-1,1′-biphenyl-4-yl)carbonyl]-N-methyl-L-valine,
N-[(4′-{[(3,5-difluorophenyl)-acetyl]amino-1,1′-biphenyl-4-yl}carbonyl]-N-methyl-L-alanine,
N-[(4′-{[(3-chlorophenyl)-acetyl]amino}-1,1′-biphenyl-4-yl)carbonyl]-L-valine,
N-[(4′-{[(4-chlorophenyl)-acetyl]amino}biphenyl-4-yl) carbonyl]-N,2-dimethylalanine,
N-[(4′-{[(4-ethoxyphenyl)-acetyl]amino}-1,1′-biphenyl-4-yl)carbonyl]-L-valine,
N-[(4′-{[(5-chloro-2,3-dihydro-1H-indol-1-yl)carbonyl]amino}-1,1′-biphenyl-4-yl)carbonyl]-L-valine,
N-[(4′-{[(5-methoxy-1H-indol-2-yl)carbonyl]amino}biphenyl-4-yl)carbonyl]-L-valine,
N-[(4-{[(7-ethoxy-1-benzofuran-2-yl)carbonyl]amino}-1,1′-biphenyl-4-yl)carbonyl]-L-valine, and
N-[(4′-{[(7-methoxy-1-benzofuran-2-yl)carbonyl]amino}-1,1′-biphenyl-4-yl)carbonyl]-L-valine.

24. The compound of claim 1 selected from the group consisting of
N-[(4′-{[(ethylamino)carbonyl]amino}biphenyl-4-yl)carbonyl]-2-methylalanine,
N-{[4′-({[(2,3-dichlorophenyl)-amino]carbonyl}amino)-1,1′-biphenyl-4-yl]carbonyl}-D-valine,
N-{[4′-({[(2,3-dichlorophenyl)-carbonyl}amino)-1,1′-biphenyl-4-yl]carbonyl}-N-methyl-L-alanine,
N-{[4′-({[(2,3-dichlorophenyl)-amino]carbonyl}amino)biphenyl-4-yl]carbonyl}-2-methylalanine,
N-{[4′-({[(2,3-dimethylphenyl)-amino]carbonyl}amino)-1,1′-biphenyl-4-yl]carbonyl}-N-methyl-L-alanine,
N-{[4′-({[(2,3-dimethylphenyl)-amino]carbonyl}amino)biphenyl-4-yl]carbonyl}-2-methylalanine,
N-{[4′-({[(2,4-dichlorophenyl)-amino]carbonyl}amino)-1,1′-biphenyl-4-yl]carbonyl}-D-valine,
N-{[4′-({[(2,4-dichlorophenyl)-amino]carbonyl}amino)-1,1′-biphenyl-4-yl]carbonyl}-N-methyl-L-alanine,
N-{[4′-({[(2,4-dichlorophenyl)-amino]carbonyl}amino-1,1′-biphenyl-4-yl]carbonyl}-N-methyl-L-phenylalanine,
N-{[4′-({[(2,4-dichlorophenyl)-amino]carbonyl}amino)-1,1′-biphenyl-4-yl]carbonyl}-N-methyl-L-valine,
N-{[4′-({[(2,4-dichlorophenyl)-amino]carbonyl}amino)-1,1′-biphenyl-4-yl]carbonyl}-L-valine,
N-{[4′-({[(2,4-dichlorophenyl)-amino]carbonyl}amino)biphenyl-4-yl]carbonyl}-2-methylalanine,
N-{[4′-({[(2,4-difluorophenyl)-amino]carbonyl}amino)biphenyl-4-yl]carbonyl}-2-methylalanine,
N-{[4′-({[(2,4-difluorophenyl)-amino]carbonyl}amino)-1,1′-biphenyl-4-yl]carbonyl}-D-valine,
N-{[4′-({[(2,4-difluorophenyl)-amino]carbonyl}amino)-1,1′-biphenyl-4-yl]carbonyl}-N-methyl-L-alanine,
N-{[4′-({[(2,4-difluorophenyl)-amino]carbonyl}amino)-1,1′-biphenyl-4-yl]carbonyl}-N-methyl-L, valine,
N-{[4′-({[(2,4-difluorophenyl)-amino]carbonyl}amino)-1,1′-biphenyl-4-yl]carbonyl}-L-valine,
N-{[4′-({[(3,4-dimethylphenyl)-amino]carbonyl}amino)-1,1′-biphenyl-4-yl]carbonyl}-L-valine,
N-{[4′-({[(2,4-dimethylphenyl)-amino]carbonyl}amino)-1,1′-biphenyl-4-yl]carbonyl}-D-valine, and
N-{[4′-({[(2,4-dimethylphenyl)-amino]carbonyl}amino)-1,1′-biphenyl-4-yl]carbonyl}-N-methyl-L-alanine.

25. The compound of claim 1 selected from the group consisting of
N-{[4′-({[(2,4-dimethylphenyl)-amino]carbonyl}amino)biphenyl-4-yl]carbonyl}-2-methylalanine,
N-{[4′-({[(2,5-dichlorophenyl)-amino]carbonyl}amino)biphenyl-4-yl]carbonyl}-2-methylalanine,
N-{[4′-({[(2,6-dimethylphenyl)-amino]carbonyl}amino)biphenyl-4-yl]carbonyl}-2-methylalanine,
N-{[4′-({[(2-difluorophenyl)-amino]carbonyl}amino)-1,1′-biphenyl-4-yl]carbonyl}-N-methyl-L-phenylalanine,
N-{[4′-({[(2-chlorophenyl)-amino]carbonyl}amino)-1,1′-biphenyl-4-yl]carbonyl}-D-valine,
N-{[4′-({[(2-chlorophenyl)-amino]carbonyl}amino)-1,1′-biphenyl-4-yl]carbonyl}-N-methyl-L-alanine,
N-{[4′-({[(2-chlorophenyl)-amino]carbonyl}amino)-1,1′-biphenyl-4-yl]carbonyl}-N-methyl-L-phenylalanine,
N-{[4′-({[(2-chlorophenyl)-amino]carbonyl}amino)-1,1′-biphenyl-4-yl]carbonyl}-N-methyl-L-valine,
N-{[4′-({[(2-chlorophenyl)-amino]carbonyl}amino)-1,1′-biphenyl-4-yl]carbonyl}-L-valine,
N-{[4′-({[(2-chlorophenyl)-amino]carbonyl}amino)biphenyl-4-yl]carbonyl}-2-methylalanine,
N-{[4′-({[(2-methoxy-5-methylphenyl)-amino]carbonyl}amino)-1,1′-biphenyl-4-yl]carbonyl}-N-methyl-L-alanine,
N-{[4′-({[(2-ethoxyphenyl)-amino]carbonyl}amino)-1,1′-biphenyl-4-yl]carbonyl}-N-methyl-L-valine,
N-{[4′-({[(2-ethoxyphenyl)-amino]carbonyl}amino)-1,1′-biphenyl-4-yl]carbonyl}-D-valine,
N-{[4′-({[(2-ethoxyphenyl)-amino]carbonyl}amino)-1,1′-biphenyl-4-yl]carbonyl}-N-methyl-L-phenylalanine,
N-{[4′-({[(2-ethoxyphenyl)-amino]carbonyl}amino)biphenyl-4-yl]carbonyl}-2-methylalanine,
N-{[4′-({[(2-fluorophenyl)-amino]carbonyl}amino)-1,1′-biphenyl-4-yl]carbonyl}-D-valine,
N-{[4′-({[(2-fluorophenyl)-amino]carbonyl}amino)-1,1′-biphenyl-4-yl]carbonyl}-N-methyl-L alanine,
N-{[4′-({[(2-methoxy-5-methylphenyl)amino]carbonyl}amino)-1,1′-biphenyl-4-yl]carbonyl}-D-valine,
N-{[4′-({[(4-methoxyphenyl)-amino]carbonyl}amino)biphenyl-4-yl]carbonyl}-2-methylalanine, and
N-{[4′-({[(3,4-dichlorophenyl)-amino]carbonyl}amino 1,1′-biphenyl-4-yl]carbonyl}-D-valine.

26. The compound of claim 1 selected from the group consisting of
N-{[4′-({[(3,4-dichlorophenyl)-amino]carbonyl}amino)-1,1′-biphenyl-4-yl]carbonyl}-N-methyl-L-alanine,
N-{[4′-({[(3,4-dichlorophenyl)-amino]carbonyl}amino)-1,1′-biphenyl-4-yl]carbonyl}-N-methyl-L-valine,
N-{[4′-({[(3,4-dichlorophenyl)-amino]carbonyl}aminol)-1,1′-biphenyl-4-yl]carbonyl}-L-valine,
N-{[4′-({[(3,4-dichlorophenyl)-amino]carbonyl}amino)biphenyl-4-yl]carbonyl}-2-methylalanine,
N-{[4′-({[(3,4-difluorophenyl)-amino]carbonyl}amino)-1,1′-biphenyl-4-yl]carbonyl}-L-valine,
N-{[4′-({[(2,3-dimethylphenyl)-amino]carbonyl}amino)-1,1′-biphenyl-4-yl]carbonyl}-D-valine,
N-{[4′-({[(3,4-dimethylphenyl)-amino]carbonyl}amino)-1,1′-biphenyl-4-yl]carbonyl}-D-valine,
N-{[4′-({[(3,4-dimethylphenyl)-amino]carbonyl}amino)-1,1′-biphenyl-4-yl]carbonyl}-N-methyl-L-alanine,
N-{[4′-({[(3,4-dimethylphenyl)-amino]carbonyl}-amino)biphenyl-4-yl]carbonyl}-2-methylalanine,
N-{[4′-({[(3,5-dichlorophenyl)-amino]carbonyl}amino)-1,1′-biphenyl-4-yl]carbonyl}-D-valine,
N-{[4′-({[(3,5-dichlorophenyl)-amino]carbonyl}amino)-1,1′-biphenyl-4-yl]carbonyl}-N-methyl-L-alanine,
N-{[4′-({[(3,5-dichlorophenyl)-amino]carbonyl}amino)biphenyl-4-yl]carbonyl}-2-methylalanine,
N-{[4′-({[(3-chloro-4-methylphenyl)-amino]carbonyl}amino)-1,1′-biphenyl-4-yl]carbonyl}-D-valine,
N-{[4′-({[(3-chloro-4-methylphenyl)-amino]carbonyl}amino)-1,1′-biphenyl-4-yl]carbonyl}-N-methyl-L-alanine,
N-{[4′-({[(3-chloro-4-methylphenyl)-amino]carbonyl}amino)-1,1′-biphenyl-4-yl]carbonyl}-L-valine,
N-{[4′-({[(4-butylphenyl)-amino]carbonyl}amino)-1,1′-biphenyl-4-yl]carbonyl}-D-valine,
N-{[4′({[(4-butylphenyl)-amino]carbonyl}amino)-1,1′-biphenyl-4-yl]carbonyl}-N-methyl-L-alanine,
N-{[4′-({[(4-butylphenyl)-amino]carbonyl}amino)-1,1′-biphenyl-4-yl]carbonyl}-L-valine,
N-{[4′-({[(4-butylphenyl)-amino]carbonyl}amino)biphenyl-4-yl]carbonyl}-2-methylalanine, and
N-{[4′-({[(4-chloro-2-methylphenyl)-amino]carbonyl} amino)-1,1′-biphenyl-4-yl]carbonyl}-D-valine.

27. The compound of claim 1 selected from the group consisting of
N-{[4′-({[(4-chloro-2-methylphenyl)-amino]carbonyl}amino)-1,1′-biphenyl-4-yl]carbonyl}-N-methyl-L-alanine,
N-{[4′-({[(4-chloro-2-methylphenyl)-amino]carbonyl}amino)-1,1′-biphenyl-4-yl]carbonyl}-N-methyl-L-valine,
N-{[4′-({[(4-chloro-2-methylphenyl)-amino]carbonyl}amino)-1,1′-biphenyl-4-yl]carbonyl}-L-valine,
N-{[4′-({[(4-chloro-2-methylphenyl)-amino]carbonyl}amino)biphenyl-4-yl]carbonyl}-2-methylalanine,
N-{[4′-({[(4-chlorophenyl)-amino]carbonyl}amino)-1,1′-biphenyl-4-yl]carbonyl}-D-valine,
N-{[4′-({[(4-chlorophenyl)-amino]carbonyl}amino)-1,1′-biphenyl-4-yl]carbonyl}-N-methyl-L-alanine,
N-{[4′-({[(4-chlorophenyl)-amino]carbonyl}amino)-1,1′-biphenyl-4-yl]carbonyl}-N-methyl-L-valine,
N-{[4′-({[(4-chlorophenyl)-amino]carbonyl}amino)-1,1′-biphenyl-4-yl]carbonyl}-L-valine,
N-{[4′-({[(4-chlorophenyl)-amino]carbonyl}amino)biphenyl-4-yl]carbonyl}-2-methylalanine,
N-{[4′-({[(4-ethoxyphenyl-amino]carbonyl}amino)-1,1′-biphenyl-4-yl]carbonyl}-D-valine,
N-{[4′-({[(2-ethoxyphenyl)-amino]carbonyl}amino)-1,1′-biphenyl-4-yl]carbonyl}-N-methyl-L, alanine,
N-{[4′-({[(4-ethoxyphenyl)-amino]carbonyl}amino)-1,1′-biphenyl-4-yl]carbonyl}-N-methyl-L-alanine,
N-{[4′-({[(2-ethoxyphenyl)-amino]carbonyl}amino)-1,1′-biphenyl-4-yl]carbonyl}-L-valine,
N-{[4′-({[(4-ethylphenyl)-amino]carbonyl}amino)-1,1′-biphenyl-4-yl]carbonyl}-D-valine,
N-{[4′-({[(4-ethylphenyl)-amino]carbonyl}amino)-1,1′-biphenyl-4-yl]carbonyl}-N-methyl-L-alanine,
N-{[4′-({[(4-ethylphenyl)-amino]carbonyl}amino)-1,1′-biphenyl-4-yl]carbonyl}-N-methyl-L-valine,
N-{[4′-({[(4-fluorobenzyl)amino]carbonyl}amino)biphenyl-4-yl]carbonyl}-2-methylalanine,
N-{[4′-({[(4-fluorophenyl)-amino]carbonyl}amino)-1,1′-biphenyl-4-yl]carbonyl}-D-valine,
N-{[4′-({[(4-fluorophenyl)-amino]carbonyl}amino)-1,1′-biphenyl-4-yl]carbonyl}-N-methyl-L-alanine, and
N-{[4′-({[(4-fluorophenyl)-amino]carbonyl}amino)-1,1′-biphenyl-4-yl]carbonyl}-L-valine.

28. The compound of claim 1 selected from the group consisting of
N-{[4′-({[(4-isopropylphenyl)-amino]carbonyl}amino)biphenyl-4-yl]carbonyl}-2-methylalanine,
N-{[4′-({[(4-methoxy-2-methylphenyl)-amino]carbonyl}amino)-1,1′-biphenyl-4-yl]carbonyl}-D-valine,
N-{[4′-({[(4-methoxy-2-methylphenyl)-amino]carbonyl}amino)-1,1′-biphenyl-4-yl]carbonyl}-N-methyl-L-alanine,
N-{[4′-({[(4-meth(oxy-2-methylphenyl)-amino]carbonyl}amino)biphenyl-4-yl]carbonyl}-2-methylalanine,
N-{[4′-({[(4-methylphenyl)-amino]carbonyl}amino)-1,1′-biphenyl-4-yl]carbonyl}-N-methyl-L-valine,
N-{[4′-({[(4-methylphenyl)-amino]carbonyl}amino)biphenyl-4-yl]carbonyl}-2-methylalanine,
N-{[4′-(pentanoylamno)-1,1′-biphenyl-4-yl]carbonyl}-L-valine, and
N-methyl-N-{[4′-(pentanoylamino)-1,1′-biphenyl-4-yl]carbonyl}-N-methyl-L-valine.

29. The compound of claim 1 selected from the group consisting of
1-{[4′-({[(3,4-dimethylphenyl)-amino]carbonyl}amino)-1,1′-biphenyl-4-yl]sulfonyl}-L-proline,
1-{[4′-({[(2,4-dichlorophenyl)amino]carbonyl}amino)-1,1′-biphenyl-4-yl]sulfonyl}-L-proline,
1-{[4-({[(2,4-difluorophenyl)-amino]carbonyl}amino)-1,1′-biphenyl-4-yl]sulfonyl}-L-proline,
1-{[4′-({[(2,4-dimethylphenyl)-amino]carbonyl}amino)-1,1′-biphenyl-4-yl]sulfonyl}-proline,
1-{[4′-({[(2-chlorophenyl)-amino]carbonyl}amino)-1,1′-biphenyl-4-yl]sulfonyl}-L-proline,
1-{[4′-({[(2-ethoxyphenyl)-amino]carbonyl}amino)-1,1′-biphenyl-4-yl]sulfonyl}-L-proline,
1-{[4′-({[(3,4-dichlorophenyl)-amino]carbonyl}amino)-1,1′-biphenyl-4-yl]sulfonyl}-L-proline,
1-{[4′-({[(3,4-difluorophenyl)-amino]carbonyl}amino)-1,1′-biphenyl-4-yl]sulfonyl}-L-proline,
1-{[4′-({[(4-butylphenyl)-amino]carbonyl}amino)-1,1′-biphenyl-4-yl]sulfonyl}-L-proline,
1-{[4′-({[(4-chloro-2-methylphenyl)-amino]carbonyl}amino)-1,1′-biphenyl-4-yl]sulfonyl}-L-proline,
1-{[4-({[(4-chlorophenyl)-amino]carbonyl}amino)-1,1′-biphenyl-4-yl]sulfonyl}-L-proline,
1-{[4′-({[(4-ethylphenyl)-amino]carbonyl}amino-1,1′-biphenyl-4-yl]sulfonyl}-L-proline,
N-({4′-[(4-chlorobenzoyl)amino]-1,1′-biphenyl-4-yl}sulfonyl)-L-valine,
N-({4′-[(2-fluorobenzoyl)amino]-1,1′-biphenyl-4-yl}sulfonyl)-L-valine,
N-({4′-[(3,4-dichlorobenzoyl)amino]-1,1′-biphenyl-4-yl}sulfonyl)-N-methyl-L-valine,
N-({4′-[(2-fluorobenzoyl)amino]-1,1′-biphenyl-4-yl}sulfonyl)-N-methyl-L-valine,
N-({4′-[(3,4-difluorobenzoyl)amino]-1,1′-biphenyl-4-yl}sulfonyl)-L valine,
N-({4′-[(3,4-dimethylbenzoyl)amino]-1,1′-biphenyl-4-yl}sulfonyl)-L-valine,
N-({4′-[(3,4-dimethylbenzoyl)amino]-1,1′-biphenyl-4-yl}sulfonyl)-N-methyl-L-valine, and
N-({4′-[(3,5-difluorobenzoyl)amino]-1,1′-biphenyl-4-yl}sulfonyl)-L-valine.

30. The compound of claim 1 selected from the group consisting of
N-({4′-[(3,5-dimethoxybenzoyl)amino]-1,1′-biphenyl-4-yl}sulfonyl)-L-valine,
N-({4′-[(3-methylbutanoyl)amino]-1,1′-biphenyl-4-yl}sulfonyl)-L-valine,
N-({4′-[(4-butylbenzoyl)amino]-1,1′-biphenyl-4-yl}sulfonyl)-L-valine,
N-({4′-[(4-ethylbenzoyl)amino]-1,1′-biphenyl-4-yl}sulfonyl)-L-valine,
N-({4′-[(4-butylbenzoyl)amino]-1,1′-biphenyl-4-yl}sulfonyl)-N-methyl-L-valine,
N-({4′-[(4-chlorobenzoyl)amino]-1,1′-biphenyl-4-yl}sulfonyl)-N-methyl-L-valine,
N-({4′-[(4-fluoro-3-methylbenzoyl)amino]-1,1′-biphenyl-4-yl}sulfonyl)-L-valine,
N-({4′-[(4-fluoro-3-methylbenzoyl)amino]-1,1′-biphenyl-4-yl}sulfonyl)-N-methyl-L-valine,
N-({4′-[(4-fluorobenzoyl)amino]-1,1′-biphenyl-4-yl}sulfonyl)-L-valine,
N-({4′-[(4-fluorobenzoyl)amino]-1,1′-biphenyl-4-yl}sulfonyl)-N-methyl-L-valine,
N-({4′-[(4-methylpentanoyl)amino]-1,1′-biphenyl-4-yl}sulfonyl)-L-valine,
N-[(4′-{[(3,4-dimethoxyphenyl)-acetyl]amino}-1,1′-biphenyl-4-yl)sulfonyl]-L-valine,
N-[(4′-{[(3,5-difluorophenyl)acetyl]amino}-1,1′-biphenyl-4-yl)sulfonyl]-L-valine,
N-[(4′-({[(3,5-difluorophenyl)-acetyl]amino}-1,1′-biphenyl-4-yl)sulfonyl]-N-methyl-L-valine,
N-{[4′-({[(2,4-dichlorophenyl)-amino]carbonyl}amino)-1,1′-biphenyl-4-yl]sulfonyl}-L-valine,
N-{[4′-({[(4-difluorophenyl)-amino]carbonyl}amino)-1,1′-biphenyl-4-yl]sulfonyl}-L-valine,
N-{[4′-({[(2-chlorophenyl)-amino]carbonyl}amino)-1,1′-biphenyl-4-yl]sulfonyl}-L-valine,
N-{[4′-({[(2-ethoxyphenyl)-amino]carbonyl}amino)-1,1′-biphenyl-4-yl]sulfonyl}-L-valine,
N-{[4′-(pentanoylamino)-1,1′-biphenyl-4-yl]sulfonyl}-L-valine, and
N-methyl-N-{4′-[(4-methylpentanoyl)amino]-1,1′-biphenyl-4-yl}sulfonyl)-L-valine.

31. A pharmaceutical composition comprising a therapeutically effective amount of a compound of any of claims 1 to 30, or a pharmaceutically acceptable salt or ester, in combination with a pharmaceutically acceptable carrier.

32. A pharmaceutical composition comprising a therapeutically effective amount of a compound of any of claims 1 to 30, or a pharmaceutically acceptable salt or ester thereof, in combination with a pharmaceutically acceptable carrier and one or more pharmaceutical agents.

33. The pharmaceutical composition of claim 32, wherein said pharmaceutical agent is an anti-obesity agent selected from the group consisting of β-3 agonists, CB-1 antagonists, neuropeptide Y5 inhibitors, appetite suppressants, and lipase inhibitors.

34. The pharmaceutical composition of claim 32, wherein said pharmaceutical agent is an agent for the treatment of diabetes selected from the group consisting of insulin, insulin derivatives, PPAR ligands, sulfonylurea drugs, α-glucosidase inhibitors, biguanides, MP-1B inhibitors, DPP-IV inhibitors, 11-beta-HSD inhibitors, GLP-1 and GLP-1 derivatives, GIP and GIP derivatives, PACAP and PACAP derivatives, and secretin and secretin derivatives.

35. The pharmaceutical composition of claim 32, wherein said pharmaceutical agent is an agent for the treatment of lipid disorders selected from the group consisting of HMG-CoA inhibitors, nicotinic acid, fatty acid lowering compounds, lipid lowering drugs, ACAT inhibitors, bile sequestrants, bile acid reuptake inhibitors, microsomal triglyceride transport inhibitors, and fibric acid derivatives.

36. The pharmaceutical composition of claim 32, wherein said pharmaceutical agent is an anti-hypertensive agent selected from the group consisting of O-blockers, calcium channel blockers, diuretics, renin inhibitors, ACE inhibitors, AT-1 receptor antagonists, ET receptor antagonists, and nitrates.

37. A method of treating obesity comprising the step of administering to a subject in need thereof a therapeutically effective amount of a compound of any of claims 1 to 30 or a composition of claim 31.

38. A method of inducing weight loss comprising the step of administering to a subject in need thereof a therapeutically effective amount of a compound of any of claims 1 to 30 or a composition of claim 31.

39. A method of preventing weight gain comprising the step of administering to a subject in need thereof a therapeutically effective amount of a compound of any of claims 1 to 30 or a composition of claim 3i.

40. A method of treating obesity-related disorders comprising the step of administering to a subject in need thereof a therapeutically effective amount of a compound of any of claims 1 to 30 or a composition of claim 31.

41. The method of claim 40, wherein said obesity-related disorder is selected from the group consisting of dyslipidemia, cholesterol gallstones, gallbladder disease, gout, cancer, menstrual abnormalities, infertility, polycystic ovaries, osteoarthritis, sleep apnea, hypertriglyceridemia, Syndrome X, type 2 diabetes, atherosclerotic diseases, hyperlipidemia, hypercholesteremia, low HDL levels, hypertension, cardiovascular disease, coronary heart disease, coronary artery disease, cerebrovascular disease, stroke, and peripheral vessel disease.

42. A method of treating obesity comprising the step of administering to a subject in need thereof a therapeutically effective amount of a compound of any of claims 1 to 30 in combination with one or more pharmaceutical agents.

43. The method of claim 42, wherein said pharmaceutical agent is an anti-obesity agent selected from the group consisting of P-3 agonists, CB-1 antagonists, neuropeptide Y5 inhibitors, appetite suppressants, and lipase inhibitors.

44. The method of claim 42, wherein said pharmaceutical agent is an agent for the treatment of diabetes selected from the group consisting of insulin, insulin derivatives, PPAR ligands, sulfonylurea drugs, α-glucosidase inhibitors, biguanides, PTP-1B inhibitors, DPP-IV inhibitors, 11-beta-HSD inhibitors, GLP-1 and GLP-1 derivatives, GIP and GIP derivatives, PACAP and PACAP derivatives, and secretin and secretin derivatives.

45. The method of claim 42, wherein said pharmaceutical agent is an agent for the treatment of lipid disorders selected from the group consisting of HMG-CoA inhibitors, nicotinic acid, fatty acid lowering compounds, lipid lowering drugs, ACAT inhibitors, bile sequestrants, bile acid reuptake inhibitors, microsomal triglyceride transport inhibitors, and fibric acid derivatives.

46. The method of claim 42, wherein said pharmaceutical agent is an anti-hypertensive agent selected from the group consisting of β-blockers, calcium channel blockers, diuretics, renin inhibitors, ACE inhibitors, AT-1 receptor antagonists, ET receptor antagonists, and nitrates.

47. The method of claim 42, wherein the compound of claim 1 and one or more pharmaceutical agents are administered as a single pharmaceutical dosage formulation.

48. Compounds according to any of claims 1 to 30 for the treatment and/or prophylaxis of obesity and obesity-related disorders.

49. Medicament containing at least one compound according to any of claims 1 to 30 in combination with at least one pharmaceutically acceptable, pharmaceutically safe carrier or excipient.

50. Use of compounds according to any of claims 1 to 30 for manufacturing a medicament for the treatment and/or prophylaxis of obesity and obesity-related disorders.

51. Medicaments according to claim 49 for the treatment and/or prophylaxis of obesity.
